(19) United States
(12) Patent Application Publication
(10) Pub. No.: US 2023/0089014 A1
(43) Pub. Date: Mar. 23, 2023

(54) COMPOUND USED AS KINASE INHIBITOR AND USE THEREOF

(71) Applicants: TYK MEDICINES (ZHENGZHOU), INC., Henan (CN); TYK MEDICINES, INC., Zhejiang (CN)

(72) Inventors: Jun LI, Henan (CN); Apeng LIANG, Henan (CN); Chengshan NIU, Henan (CN); Yusheng WU, Henan (CN)

(73) Assignees: TYK MEDICINES (ZHENGZHOU), INC., Henan (CN); TYK MEDICINES, INC., Zhejiang (CN)

(21) Appl. No.: 17/787,940

(22) PCT Filed: Apr. 16, 2021
(86) PCT No.: PCT/CN2021/087725

§ 371 Date: Jun. 22, 2022

(30) Foreign Application Priority Data
Feb. 19, 2020 (CN) 202010102525.1

(51) Int. Cl. C07D 401/06; C07D 239/94; C07D 405/14; C07D 471/08; A61K 31/517

(52) U.S. Cl. CPC C07D 401/06; C07D 239/94; C07D 405/14; C07D 471/08; A61K 31/517

(57) ABSTRACT

The present invention relates to a compound used as a kinase inhibitor and to the use thereof. The structure of the compound is as shown in formula I. The present compound used as a kinase inhibitor has good inhibitory activity on EGFR and Her2 exon 20 insertion mutations, and has excellent potential to be developed into a drug for treating related diseases.

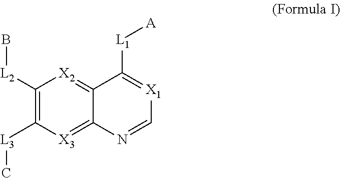

### TECHNICAL FIELD

The disclosure relates to the field of nitrogen-containing heterocyclic compounds, in particular to a compound used as a kinase inhibitor and use thereof.

### DESCRIPTION OF RELATED ART

Epidermal growth factor (EGF) receptors belong to the receptor tyrosine kinase (RTK) family, which includes EGFR/ERBB1, HER2/ERBB2/NEU, HER3/ERBB3 and HER4/ERBB4. Epidermal growth factor receptor (EGFR) activates its tyrosine kinase activity through homodimerization or heterodimerization, which in turn phosphorylates its substrates, thereby activating multiple downstream pathways associated with it in cells, such as PI3K-AKT-mTOR pathway involved in cell survival and RAS-RAF-MEK-ERK pathway involved in cell proliferation. Mutation or augmentation of EGFR can lead to the activation of EGFR kinase, which leads to the occurrence of various human diseases, such as malignant tumors. For example, among patients with non-small-cell lung cancer, more than 10% of patients in the United States have EGFR mutations, while the proportion of patients with EGFR mutations in Asian patients can reach nearly 50%. Meanwhile, in patients with non-small-cell lung cancer, the incidence of Her2 mutation is about 2-4%.

EGFR mutations mainly include deletions, insertions and point mutations, among which exon 19 deletion and exon 21 L858R point mutation account for nearly 90% of EGFR mutations. For tumor patients with these EGFR mutations, currently available EGFR-TKIs include the first-generation Iressa, Tarceva, and Conmena, the second-generation afatinib and dacomitinib, and the third-generation osimertinib. The other 10% of EGFR mutations mainly involve EGFR exons 18 and 20, and insertion mutations of EGFR exon 20 account for about 9% of all EGFR mutations. For tumor patients with Her2 mutations, the most common Her2 mutation is an insertion mutation in Her2 exon 20. For EGFR and Her2 exon 20 insertion mutations, there are no drugs available on the market.

Patent document WO2008150118A2 discloses that a series of quinazoline derivatives have activity of EGFR T790M drug resistance mutation. Meanwhile, the patent document also discloses that this series of compounds has good biological activity against skin cancer cell line A431 (this cell line overexpresses WT EGFR) and breast cancer cell line SK-Br3 (this cell line overexpresses Her2). There is no report about activity of EGFR and Her2 exon 20 insertion mutation.

The typical compound involved in this patent is the compound (Poziotinib) shown in formula II:

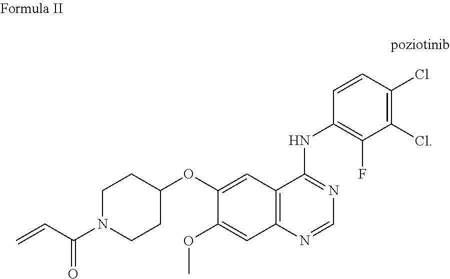 [SMILES: C=CC(=O)N1CCC(Oc2cc3c(Nc4ccc(Cl)c(Cl)c4F)ncnc3cc2OC)CC1]

The compound (Poziotinib) shown in formula II was reported to have failed clinical development directed at EGFR exon 19 deletion and exon 21 L858R point mutation. However, the compound (Poziotinib) was found to be clinically effective in some patients with EGFR exon 20 insertion, and therefore the compound (Poziotinib) turned out to be used in clinical development directed at EGFR exon 20 insertion mutations. However, because the compound (Poziotinib) also has very good inhibitory activity against wild-type EGFR, the therapeutic window of the medication is too small, and it is very likely that the toxic side effects will be relatively dramatic (Signal Transduction and Targeted Therapy, 2019, 4:5). As a result, the effective therapeutic dose of the medication cannot be increased, which affects the curative effect. At present, this compound is still under clinical research.

### SUMMARY

The purpose of the present disclosure is to provide a compound used as a kinase inhibitor to solve the problem that the existing inhibitors have poor inhibitory activity against EGFR and Her2 exon 20 insertion mutations.

The second purpose of the present disclosure is to provide the application of the above-mentioned compound in the preparation of medication, which may be used for the treatment of related diseases caused by EGFR mutation and/or Her2 mutation.

To achieve the above purposes, the technical solution of the compound used as a kinase inhibitor of the present disclosure is stated below.

The compound used as a kinase inhibitor is a compound shown in formula I, or an isomer thereof, or a pharmaceutically acceptable salt, solvate or prodrug thereof;

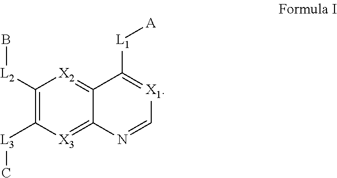

In formula I, X₁ is selected from N or CR₂; X₂ is selected from N or CR₃; X₃ is selected from N or CR₄.

L₁ and L₃ are each independently selected from a single bond,

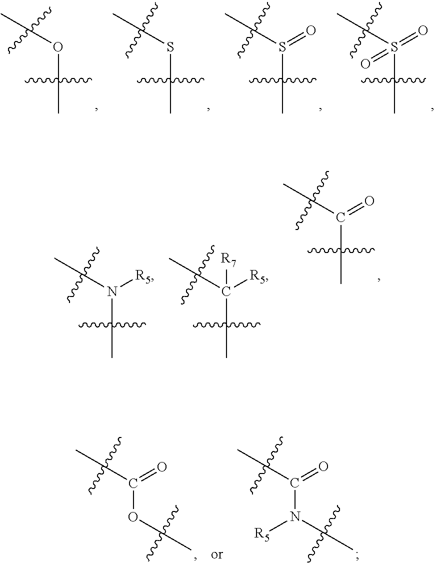

L₂ is selected from a single bond,

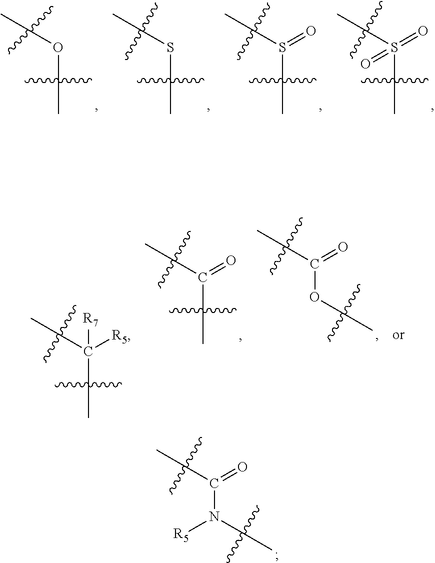

A is selected from C₆₋₁₀ aryl and C₅₋₁₂ heteroaryl; or A is C₆₋₁₀ aryl substituted with 1, 2 or 3 substituents or C₅₋₁₂ heteroaryl substituted with 1, 2 or 3 substituents. The substituent is selected from any one of H, halogen, cyano, amino, ester, urea, carbamate, amide, C₁₋₆ alkyl, C₁₋₆ alkoxy, C₃₋₆ cycloalkyl, C₃₋₆ cycloalkoxy, C₆₋₁₀ aryl, and C₅₋₁₂ heteroaryl; or the substituents is amino group, ester group, urea group, carbamate group, amide group, C₁₋₆ alkyl group, C₁₋₆ alkoxy group, C₃₋₆ cycloalkyl group, C₃₋₆ cycloalkoxy group, C₆₋₁₀ aryl, and C₅₋₁₂ heteroaryl, which is substituted with 1, 2 or 3 R.

R is selected from halogen, cyano, hydroxyl, amino, ester, urea, carbamate, amide, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆₋₁₀ aryl, and C₅₋₁₂ heteroaryl.

B is a nitrogen-containing heterocyclic group or a nitrogen-containing heterocyclic group substituted by R₁, and the number of nitrogen heteroatoms in the nitrogen-containing heterocyclic group is one or more.

R₁ is selected from

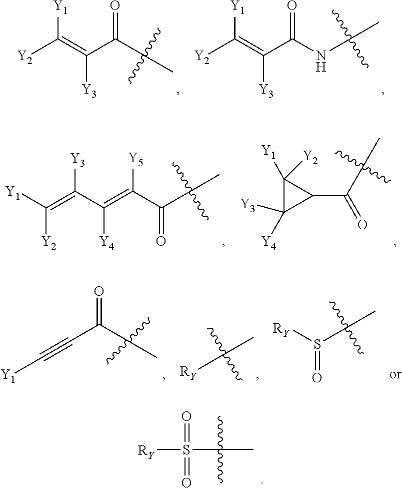

Y₁, Y₂, Y₃, Y₄, and Y₅ are each independently selected from hydrogen, halogen, C₁₋₁₂ alkyl, C₃₋₁₂ cycloalkyl, and C₁₋₁₂ alkylamino; or Y₁, Y₂, Y₃, Y₄, and Y₅ are each independently C₁₋₁₂ alkyl, C₃₋₁₂ cycloalkyl, or C₁₋₁₂ alkylamino, which is substituted by the R. R_{Y} is selected from C₁₋₁₂ alkyl, C₁₋₁₂ alkyl substituted by the R, C₃₋₁₂ cycloalkyl, and C₃₋₁₂ cycloalkyl substituted by the R; or R_{Y} is C_{1- 12} alkyl, C₁₋₁₂ alkyl substituted by the R, C₃₋₁₂ cycloalkyl group, or a group formed by replacing one or more carbon atoms in the C₃₋₁₂ cycloalkyl group substituted by the R with one or more heteroatoms in N, O, and S.

R₂, R₃, R₄, R₅, R₆ and R₇ are each independently selected from H, halogen, cyano, amino, ester, urea, carbamate, amide, C₁₋₆ alkyl, C₁₋₆ alkoxy, C_{3- 6} cycloalkyl, C₃₋₆ cycloalkoxy, C₆₋₁₀ aryl, and C₅₋₁₂ heteroaryl; or R₂, R₃, R₄, R₅, R₆ and R₇ are each independently amino group, ester group, urea group, carbamate group, amide group, C₁₋₆ alkyl group, C₁₋₆ alkoxy group, C₃₋₆ cycloalkyl group, C₃₋₆ cycloalkoxy group, C₆₋₁₀ aryl, or C₅₋₁₂ heteroaryl, which is substituted with the 1, 2 or 3 R.

When L₂ is selected from

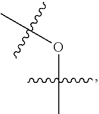 [SMILES: *O*]

B is selected from

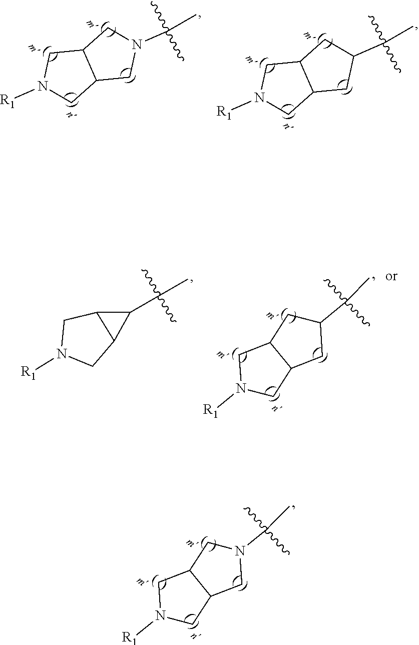

When L₂ is selected from

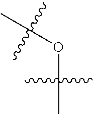 [SMILES: *O*]

and B is selected from

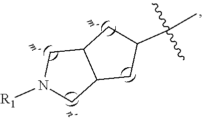

A is selected from

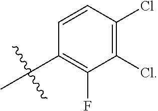 [SMILES: *c1ccc(Cl)c(Cl)c1F]

m, n, m′ and n′ are each independently selected from 0, 1, 2, and 3.

C is selected from H, halogen, cyano, amino, ester, urea, ether, carbamate, amide, C₁₋₆ alkyl, C₁₋₆ alkoxy, C₃₋₆ cycloalkyl, C₃₋₆ cycloalkoxy, C₆₋₁₀ aryl, C₅₋₁₂ heteroaryl, and aliphatic heterocycle; or C is amino group, ester group, urea group, ether group, carbamate group, amide group, C₁₋₆ alkyl group, C₁₋₆ alkoxy group, C₃₋₆ cycloalkyl group, C₃₋₆ cycloalkoxy group, C₆₋₁₀ aryl, C₆₋₁₀ heteroaryl, or aliphatic heterocycle, which is substituted by the 1, 2 or 3 R.

The compound used as a kinase inhibitor provided by the disclosure has good inhibitory activity on EGFR and Her2 exon 20 insertion mutations, and has great potential to be developed into medicines for treating related diseases.

In order to further optimize the inhibitory effect on EGFR and Her2 mutations, preferably, when L₂ is selected from a single bond,

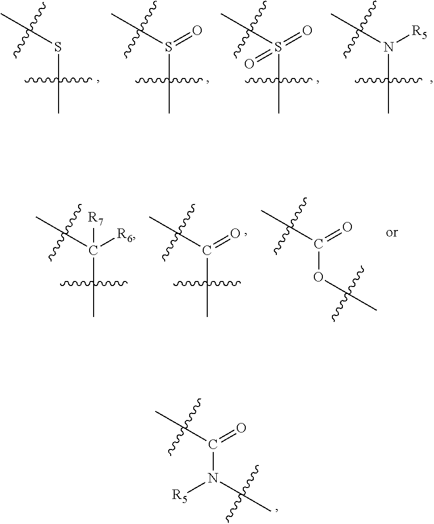

B is selected from:

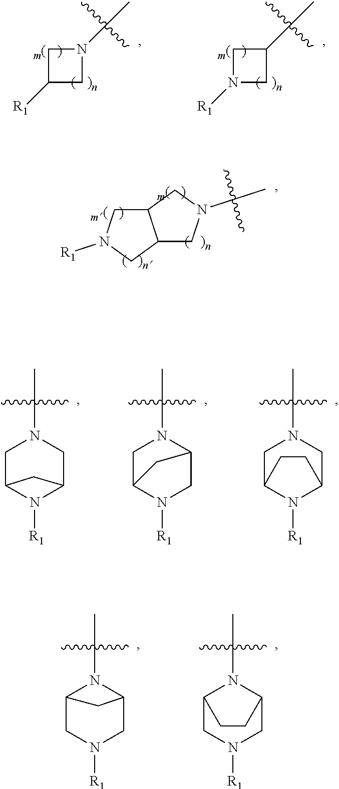

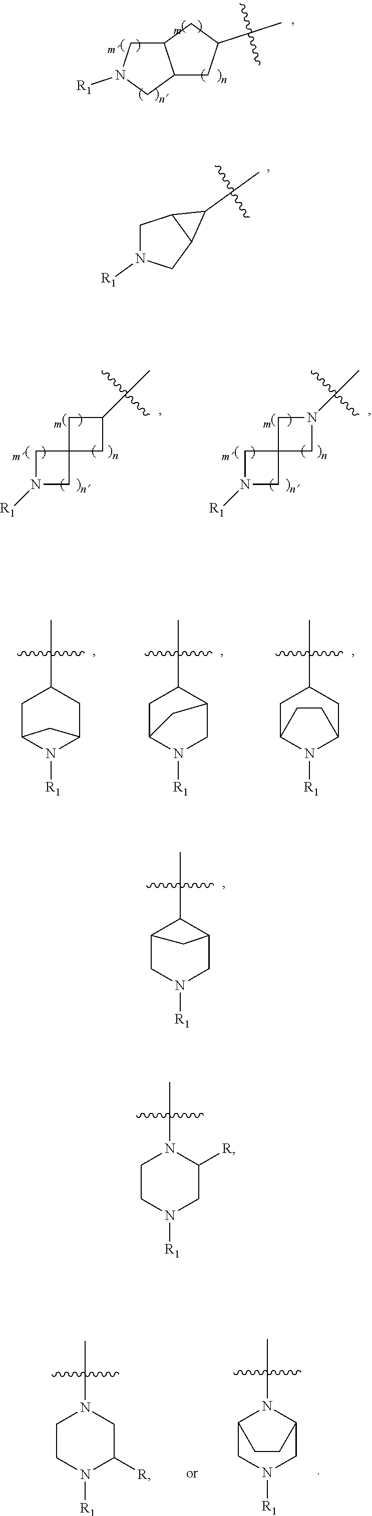

More preferably, R₁ is selected from

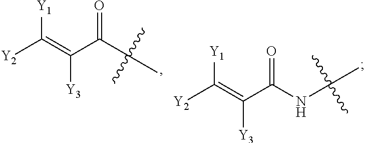

X₂ and X₃ are selected from CH; X₁ is selected from N; L₃ is selected from

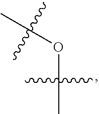 [SMILES: *O*]

C is selected from C₁₋₃ alkyl,

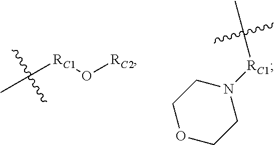

L₁ is selected from

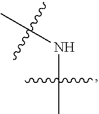 [SMILES: *N*]

A is selected from

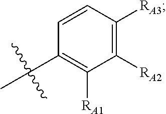

Y₁, Y₂ and Y₃ are selected from hydrogen; R_{A1}, R_{A2} and R_{A3} are each independently selected from hydrogen, halogen and C₁₋₃ alkyl; R_{C1} and R_{C2} are each independently selected from C₁₋₃ alkyl.

Preferably, L₂ is selected from

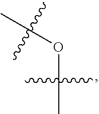 [SMILES: *O*]

B is selected from:

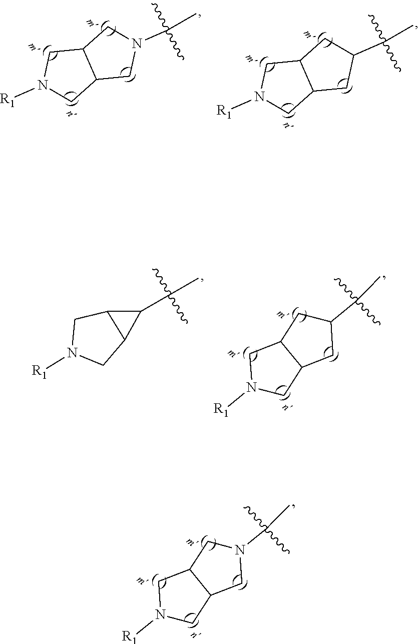

R₁ is selected from

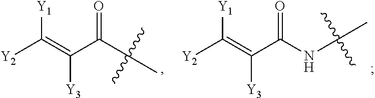

X₂ and X₃ are selected from CH ; X₁ is selected from N; L₃ is selected from

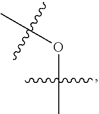 [SMILES: *O*]

C is selected from C₁₋₃ alkyl,

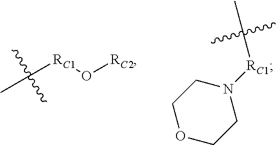

L₁ is selected from

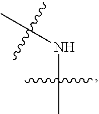 [SMILES: *N*]

A is selected from

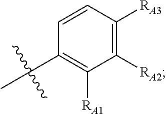

Y₁, Y₂ and Y₃ are selected from hydrogen; R_{A1}, R_{A2} and R_{A3} are each independently selected from hydrogen, halogen and C₁₋₃ alkyl; R_{C1} and R_{C2} are each independently selected from C₁₋₃ alkyl.

To further optimize the inhibitory effect on EGFR and Her2 mutations, preferably, when L₂ is selected from

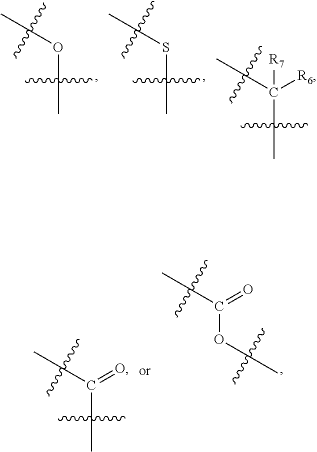

the nitrogen heteroatom of the nitrogen-containing heterocycle in B is connected to R₁.

Preferably, L₂ is a single bond, and the nitrogen heteroatom in B is connected to the parent ring. The parent ring is the structure

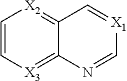

in formula I.

Preferably, L₃ is

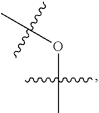 [SMILES: *O*]

C is a six-membered heterocycle, and the heteroatom in the six-membered heterocycle is N and/or O.

Preferably, the compound has the structure shown in formula II:

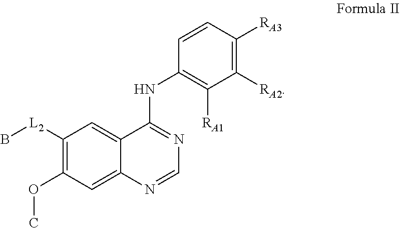

L₂ is selected from

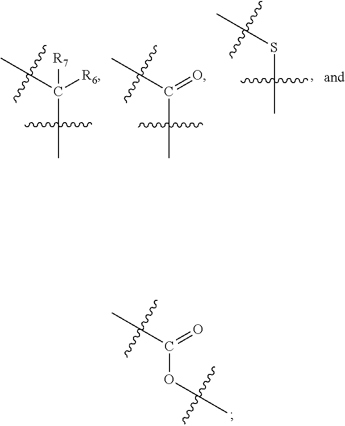

B is selected from

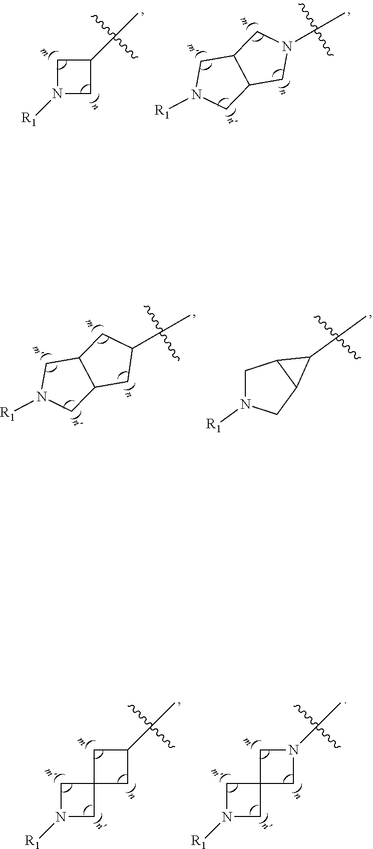

L₂ is a single bond; B is selected from

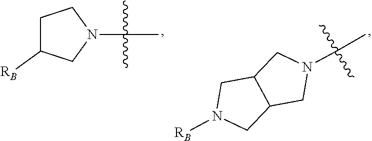

R_{B} is selected from H,

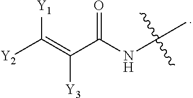

L₂ is selected from

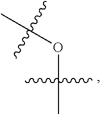 [SMILES: *O*]

B is selected from

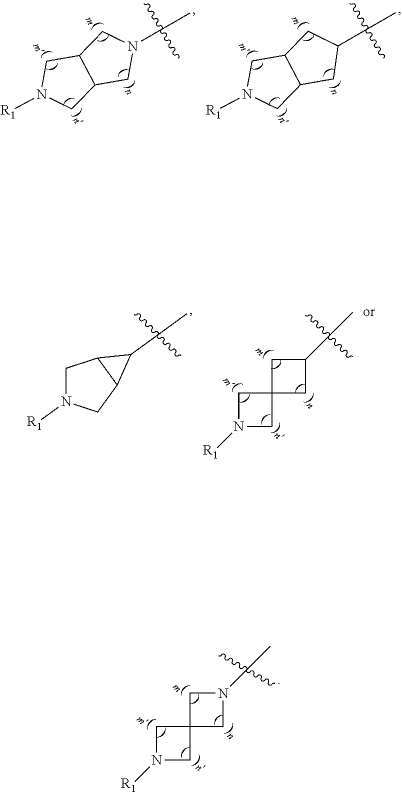

C is selected from C₁₋₃ alkyl,

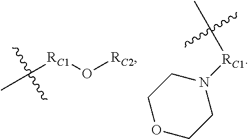

R₁ is selected from

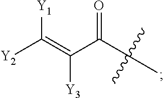

R₆ and R₇ are each independently selected from hydrogen and halogen; Y₁, Y_{2,} and Y₃ are selected from hydrogen; R_{A1}, R_{A2}, R_{A3} are each independently selected from hydrogen, halogen, and C₁₋₃ alkyl; R_{C1} and R_{C2} are each independently selected from C₁₋₃ alkyl.

More preferably, L₂ is selected from CF₂, and B is selected from

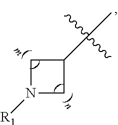

and m and n are both 1 or 2.

More preferably, L₂ is selected from

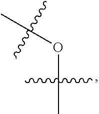 [SMILES: *O*]

and B is selected from

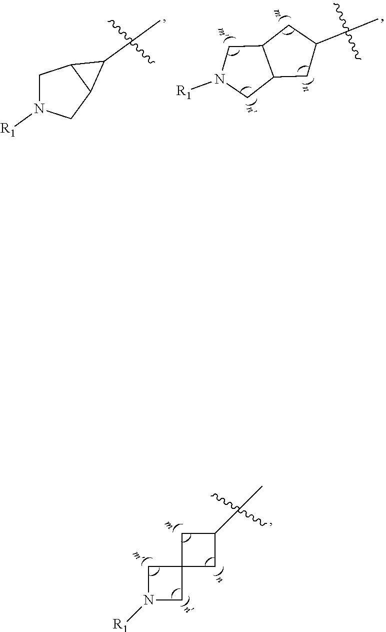

and m, n, m′, and n′ are all 1.

In order to further optimize the inhibitory effect on EGFR and Her2 mutations, preferably, the compound used as a kinase inhibitor is selected from the following compounds.

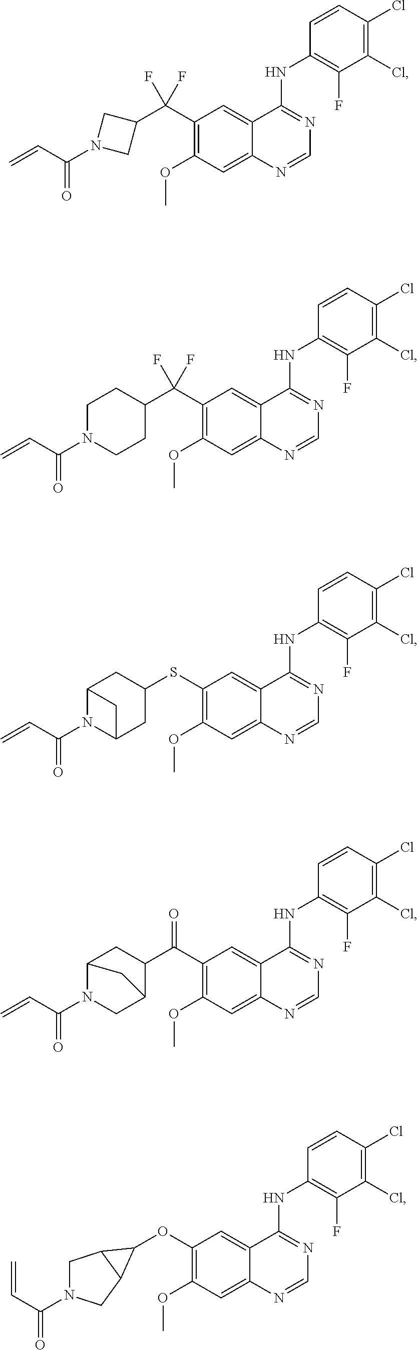 [SMILES: C=CC(=O)N1CC(C(F)(F)c2cc3c(Nc4ccc(Cl)c(Cl)c4F)ncnc3cc2OC)C1 ; C=CC(=O)N1CCC(C(F)(F)c2cc3c(Nc4ccc(Cl)c(Cl)c4F)ncnc3cc2OC)CC1 ; C=CC(=O)N1C2CC(Sc3cc4c(Nc5ccc(Cl)c(Cl)c5F)ncnc4cc3OC)CC1C2 ; C=CC(=O)N1CC2CC1CC2C(=O)c1cc2c(Nc3ccc(Cl)c(Cl)c3F)ncnc2cc1OC ; C=CC(=O)N1CC2C(C1)C2Oc1cc2c(Nc3ccc(Cl)c(Cl)c3F)ncnc2cc1OC]

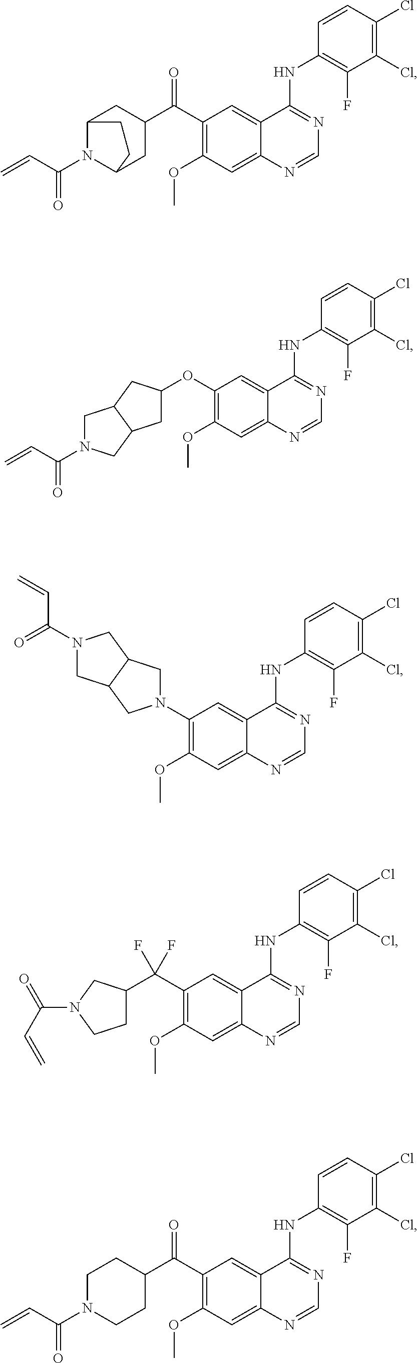 [SMILES: C=CC(=O)N1C2CCC1CC(C(=O)c1cc3c(Nc4ccc(Cl)c(Cl)c4F)ncnc3cc1OC)C2 ; C=CC(=O)N1CC2CC(Oc3cc4c(Nc5ccc(Cl)c(Cl)c5F)ncnc4cc3OC)CC2C1 ; C=CC(=O)N1CC2CN(c3cc4c(Nc5ccc(Cl)c(Cl)c5F)ncnc4cc3OC)CC2C1 ; C=CC(=O)N1CCC(C(F)(F)c2cc3c(Nc4ccc(Cl)c(Cl)c4F)ncnc3cc2OC)C1 ; C=CC(=O)N1CCC(C(=O)c2cc3c(Nc4ccc(Cl)c(Cl)c4F)ncnc3cc2OC)CC1]

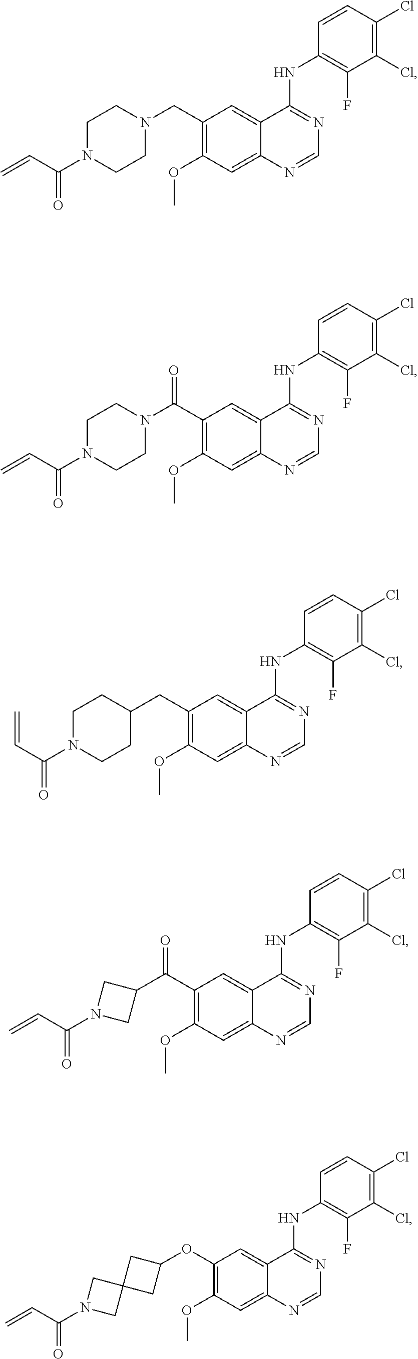 [SMILES: C=CC(=O)N1CCN(Cc2cc3c(Nc4ccc(Cl)c(Cl)c4F)ncnc3cc2OC)CC1 ; C=CC(=O)N1CCN(C(=O)c2cc3c(Nc4ccc(Cl)c(Cl)c4F)ncnc3cc2OC)CC1 ; C=CC(=O)N1CCC(Cc2cc3c(Nc4ccc(Cl)c(Cl)c4F)ncnc3cc2OC)CC1 ; C=CC(=O)N1CC(C(=O)c2cc3c(Nc4ccc(Cl)c(Cl)c4F)ncnc3cc2OC)C1 ; C=CC(=O)N1CC2(CC(Oc3cc4c(Nc5ccc(Cl)c(Cl)c5F)ncnc4cc3OC)C2)C1]

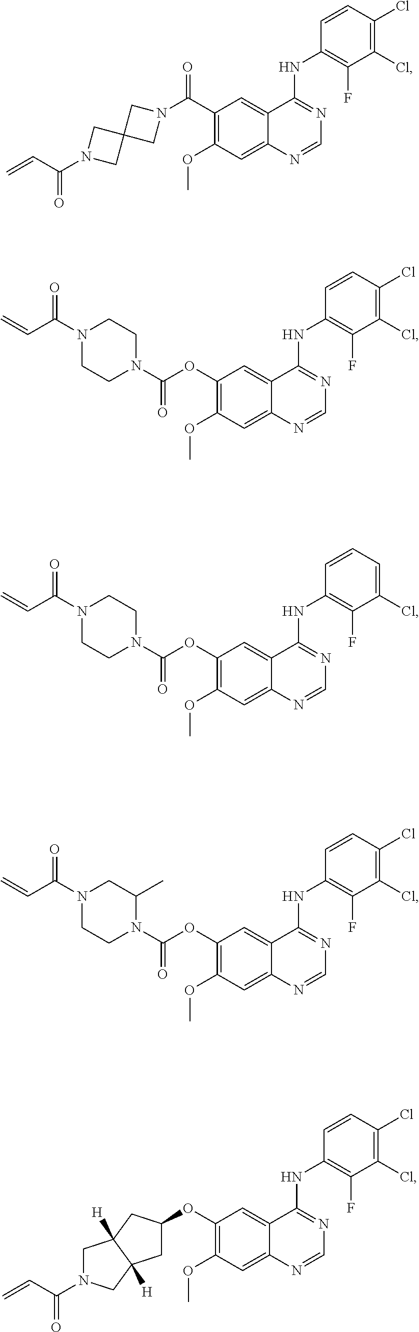 [SMILES: C=CC(=O)N1CC2(C1)CN(C(=O)c1cc3c(Nc4ccc(Cl)c(Cl)c4F)ncnc3cc1OC)C2 ; C=CC(=O)N1CCN(C(=O)Oc2cc3c(Nc4ccc(Cl)c(Cl)c4F)ncnc3cc2OC)CC1 ; C=CC(=O)N1CCN(C(=O)Oc2cc3c(Nc4cccc(Cl)c4F)ncnc3cc2OC)CC1 ; C=CC(=O)N1CCN(C(=O)Oc2cc3c(Nc4ccc(Cl)c(Cl)c4F)ncnc3cc2OC)C(C)C1 ; C=CC(=O)N1C[C@H]2C[C@H](Oc3cc4c(Nc5ccc(Cl)c(Cl)c5F)ncnc4cc3OC)C[C@H]2C1]

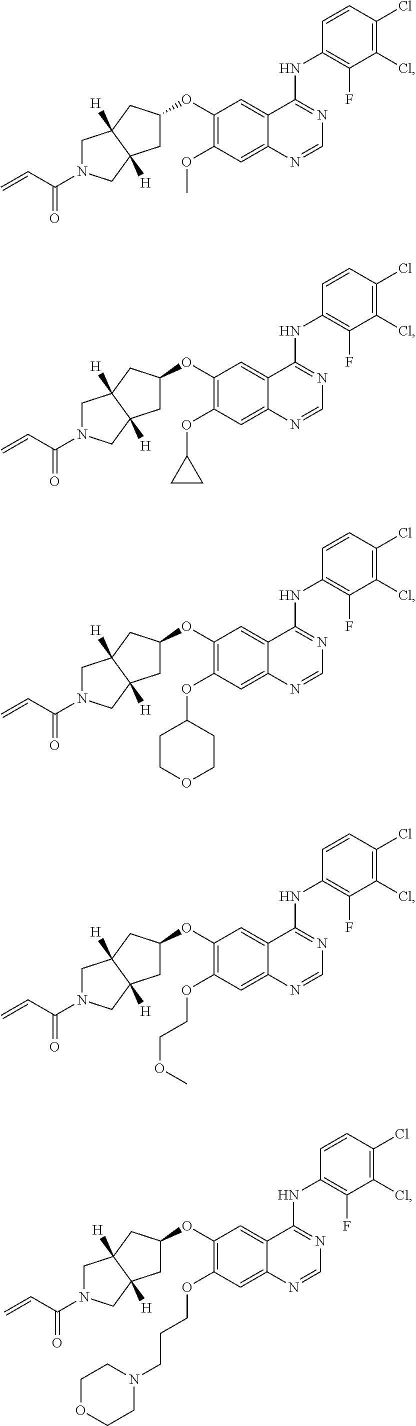 [SMILES: C=CC(=O)N1C[C@H]2C[C@@H](Oc3cc4c(Nc5ccc(Cl)c(Cl)c5F)ncnc4cc3OC)C[C@H]2C1 ; C=CC(=O)N1C[C@H]2C[C@H](Oc3cc4c(Nc5ccc(Cl)c(Cl)c5F)ncnc4cc3OC3CC3)C[C@H]2C1 ; C=CC(=O)N1C[C@H]2C[C@H](Oc3cc4c(Nc5ccc(Cl)c(Cl)c5F)ncnc4cc3OC3CCOCC3)C[C@H]2C1 ; C=CC(=O)N1C[C@H]2C[C@H](Oc3cc4c(Nc5ccc(Cl)c(Cl)c5F)ncnc4cc3OCCOC)C[C@H]2C1 ; C=CC(=O)N1C[C@H]2C[C@H](Oc3cc4c(Nc5ccc(Cl)c(Cl)c5F)ncnc4cc3OCCCN3CCOCC3)C[C@H]2C1]

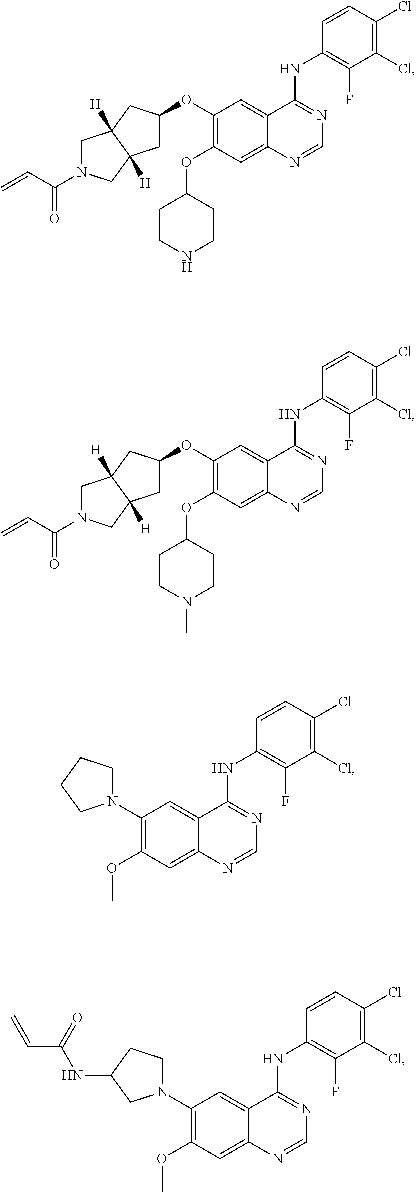 [SMILES: C=CC(=O)N1C[C@H]2C[C@H](Oc3cc4c(Nc5ccc(Cl)c(Cl)c5F)ncnc4cc3OC3CCNCC3)C[C@H]2C1 ; C=CC(=O)N1C[C@H]2C[C@H](Oc3cc4c(Nc5ccc(Cl)c(Cl)c5F)ncnc4cc3OC3CCN(C)CC3)C[C@H]2C1 ; COc1cc2ncnc(Nc3ccc(Cl)c(Cl)c3F)c2cc1N1CCCC1 ; C=CC(=O)NC1CCN(c2cc3c(Nc4ccc(Cl)c(Cl)c4F)ncnc3cc2OC)C1]

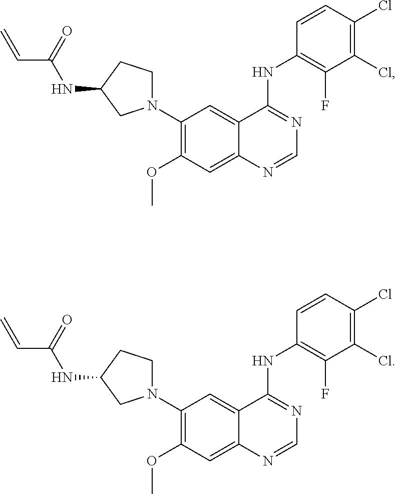 [SMILES: C=CC(=O)N[C@H]1CCN(c2cc3c(Nc4ccc(Cl)c(Cl)c4F)ncnc3cc2OC)C1 ; C=CC(=O)N[C@@H]1CCN(c2cc3c(Nc4ccc(Cl)c(Cl)c4F)ncnc3cc2OC)C1]

Unless otherwise specified, the following terms used in this specification and claims have the following meanings.

The C₆₋₁₀ aryl group is a monocyclic or bicyclic aromatic hydrocarbon group having 6 to 10 ring atoms, e.g., phenyl or naphthyl.

C₅₋₁₂ heteroaryl refers to a monocyclic or bicyclic aromatic group having 5 to 12 ring atoms; one or more, preferably one, two or three ring atoms are selected from the heteroatoms of N, O, S, and the remaining ring atoms are carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazole, quinolinyl, isoquinolinyl, pyridyl, pyrimidinyl, pyrazinyl, pyridazinyl, triazolyl, tetrazolyl and the like.

Aliphatic heterocycles are heterocyclic groups without aromatic character such as

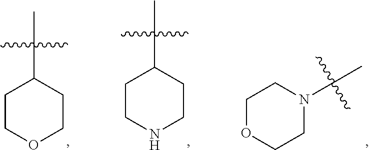 [SMILES: *C1CCOCC1 ; *C1CCNCC1 ; *N1CCOCC1]

and the like.

Alkylamino refers to a —NHR′ group; R′ refers to an alkyl group, for example, methylamino, ethylamino, propylamino, and the like.

Halogen refers to F, Cl, Br, I elements; “ [symbol] ” represents the position of chemical bond.

The above compounds have been confirmed by biological activity experiments that they have good inhibitory activity on EGFR and Her2 exon 20 insertion mutations, and may be used as the original drugs of related drugs.

On the basis of the above original drug, a “pharmaceutically acceptable salt” of the original drug refers to a salt that is pharmaceutically acceptable and possesses the desired pharmacological activity of the parent compound. Such salts include the following.

Acid addition salts formed with inorganic acids, the inorganic salts are such as hydrochloric acid, hydrobromic acid, sulfuric acid, nitric acid, phosphoric acid, etc.; or acid addition salts formed with organic acids, the organic acids are such as formic acid, acetic acid, propionic acid, caproic acid, cyclopentane propionic acid, glycolic acid, pyruvic acid, lactic acid, malonic acid, succinic acid, malic acid, maleic acid, fumaric acid, tartaric acid, citric acid, benzoic acid, 3-(4-hydroxy benzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene- 1 -carboxylic acid), 3-phenylpropionic acid, trimethylacetic acid, tert-butylacetic acid, dodecyl sulfate, gluconic acid, glutamic acid, xinafoic acid, salicylic acid, stearic acid, muconic acid, etc.; or salts formed by the coordination of the acidic proton present in the parent compound with an organic base (such as ethanolamine, diethanolamine, triethanolamine, tromethamine, N-methylglucamine, etc.). It is readily understood that the pharmaceutically acceptable salt is non-toxic.

Solvates are compounds containing a solvent, such as hydrates, dimethyl sulfoxide and the like.

A prodrug refers to a compound that undergoes chemical transformation through a metabolic or chemical process to produce the compound, salt, or solvate of the present disclosure when being used for treating a related disease.

The technical solution of the application of the above-mentioned compounds of the present disclosure is:

In the application of the compound used as a kinase inhibitor in preparing a medication, the medication may be used for the treatment of related diseases caused by EGFR mutation and/or Her2 mutation.

Based on the good inhibitory activity of the compound on EGFR and Her2 exon 20 insertion mutations, the medicines based on the compound are expected to have good therapeutic effects on related diseases.

Preferably, the EGFR mutation and Her2 mutation are exon 20 insertion mutations. The biological activity experiments confirmed that the above compound has good inhibitory effect on the insertion mutation type of exon 20 of the above two kinases.

The above compound may also be used in combination with other drugs for the treatment of cancer. Other concomitant drugs may be ERK inhibitors or MEK inhibitors. The cancer is preferably lung cancer, more preferably lung cancer caused by EGFR and Her2 exon 20 insertion mutations.

### DESCRIPTION OF THE EMBODIMENTS

The embodiments of the present disclosure will be further described below with reference to specific examples. The structure and synthesis process of the intermediate involved in the following examples are described as follows.

Synthesis of Intermediate 1:

The structural formula of intermediate 1 is as follows:

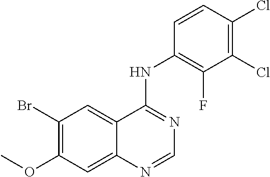 [SMILES: COc1cc2ncnc(Nc3ccc(Cl)c(Cl)c3F)c2cc1Br]

The synthetic route is as follows:

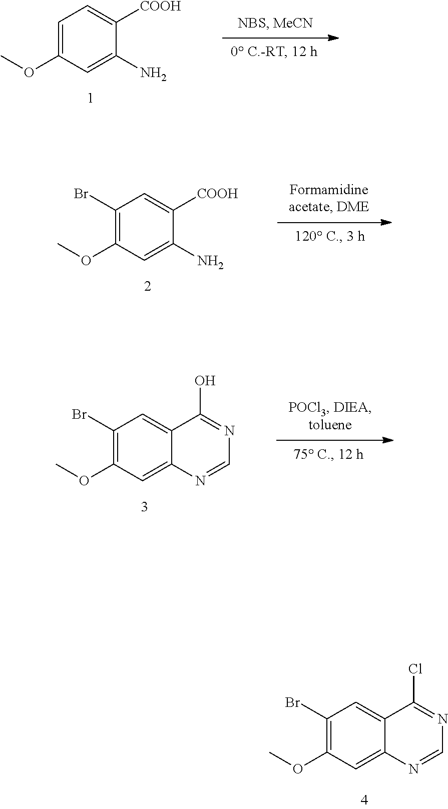

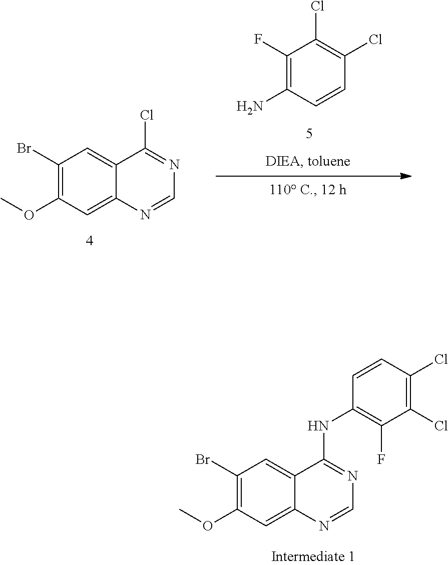 [SMILES: Nc1ccc(Cl)c(Cl)c1F ; COc1cc2ncnc(Cl)c2cc1Br ; COc1cc2ncnc(Nc3ccc(Cl)c(Cl)c3F)c2cc1Br]

The specific synthesis process is as follows:

(1) Synthesis of compound 2: A compound 1 (33.4 g, 200 mmol) and acetonitrile (MeCN, 400 mL) were added to a 250 mL three-necked flask, and cooled down to 0° C. in an ice-water bath. NBS (N-bromosuccinimide, 35.6 g, 0.2 mol) in acetonitrile solution (200 mL) was added dropwise, and was kept at 0° C. during the dropwise addition. After the dropwise addition, the temperature was increased to room temperature for reaction for 12 hours until TLC showed that the reaction was completed. After filtration, the filter cake was washed twice with 5 mL of acetonitrile, and the solvent was sucked dry to obtain 31 g of a yellow solid with a yield rate of 63%.

¹HNMR: (DMSO-d6, 400 Hz) δ: 7.77 (s, 1 H), 6.42 (s, 1 H), 3.80 (s, 3 H).

(2) Synthesis of compound 3: A compound 2 (30 g, 122 mmol), formamidine acetate (17.8 g, 171.8 mmol), and ethylene glycol dimethyl ether (DME, 30 mL) were added to a 100 mL single-neck bottle, and heated in an oil bath to an external temperature of 120° C. and refluxed for 3 hours until TLC showed that the reaction was completed. Turn off the heating. The reactant was cooled to room temperature, put in the refrigerator to cool for 0.5 hours, and filtered. The filter cake was slurried twice with 40 mL of ethyl acetate, slurried twice with 40 mL of water, and rotary evaporated to dryness to obtain 22 g of a brown solid with a yield rate of 71%.

¹HNMR: (DMSO-d6, 400 Hz) δ: 8.20 (s, 1 H), 8.12 (s, 1 H), 7.23 (s, 1 H), 4.00 (s, 3 H).

(3) Synthesis of Compound 4: A compound 3 (4 g, 15.69 mmol) and toluene (30 mL) were added to a 100 mL single-neck flask, cooled to 0° C. in an ice-water bath, and DIEA (N,N-diisopropylethylamine, 4.75 g, 36.7 mmol) was added. Phosphorus oxychloride (7.31 g, 47.1 mmol) was added dropwise, and kept at 0° C. during the dropwise addition. After the dropwise addition, the reactant was heated in an oil bath to an external temperature of 75° C., and the reaction was performed for 12 hours until TLC showed the reaction was completed (PE:EA=5:1, 3 Rf=0.5, 4 Rf=0.2). Turn off the heating. The reactant was cooled to room temperature, and poured into ice water and stirred for 10 minutes. Dichloromethane was added to separate the liquid, the aqueous phase was washed with dichloromethane twice, the organic phases were combined, and washed with saturated brine. The liquid was separated, the organic phase was evaporated to dryness. After passing through the chromatographic column, 2.3 g of a pale yellow solid with a yield rate of 55% was obtained.

¹HNMR: (DMSO-d6, 400 Hz) δ: 8.97 (s, 1 H), 8.48 (s, 1 H), 7.38 (s, 1 H), 4.10 (s, 3 H).

(4) Synthesis of compound intermediate 1: A compound 4 (3 g, 11 mmol), a compound 5 (3.96 g, 22 mmol) and toluene (30 mL) were added to a 50 mL single-neck flask, and heated in an oil bath to an external temperature of 110° C. and the reaction was carried out for 12 hours until TLC showed the reaction was completed (PE:EA=3:1, 4 Rf=0.5, 6 Rf=0.2). Turn off the heating. The reactant was cooled to room temperature, DIEA was added dropwise to make the reactant dissolved clarification. Silica gel was added, the sample was mixed and evaporated to dryness. After passing through the chromatographic column, 4.25 g of a yellow solid with a yield rate of 92.7% was obtained.

¹HNMR: (DMSO-d6, 400 Hz) δ: 8.75 (s, 1 H), 8.52˜8.48 (m, 1 H), 8.10 (s, 1 H), 7.41 (s, 1 H), 7.35˜7.31 (m, 2 H), 4.10 (s, 3 H).

Synthesis of Intermediate 2:

The structural formula of intermediate 2 is as follows:

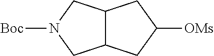 [SMILES: CC(C)(C)OC(=O)N1CC2CC(OS(C)(=O)=O)CC2C1]

The synthetic route is as follows:

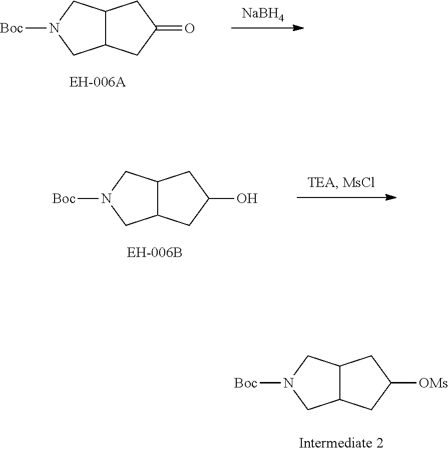 [SMILES: CC(C)(C)OC(=O)N1CC2CC(=O)CC2C1 ; "NaBH4" ; CC(C)(C)OC(=O)N1CC2CC(O)CC2C1 ; CC(C)(C)OC(=O)N1CC2CC(OS(C)(=O)=O)CC2C1]

The experiment process is as follows:

(1) A 25 mL single-necked bottle was taken and added with EH-006A (200 mg, 0.89 mmol) and methanol (6 mL), and sodium borohydride (33.8 g, 0.89 mmol) was added thereto in three batches after being cooled in an ice bath. The reaction solution was stirred at room temperature for 2 hours. TLC was carried out to detect that the raw materials were completely converted. The solvent was rotary evaporated to dryness, and water (10 mL) and ethyl acetate (10 mL) were added to the residue, stirred for 5 minutes; the liquid was then separated, and the organic layer was collected. The aqueous phase was extracted with ethyl acetate (10 mL). The combined organic phase was dried over anhydrous magnesium sulfate, and the solvent was evaporated under reduced pressure. The residue was subjected to column chromatography to obtain a colorless oil EH-006B (222 mg); the yield rate was 100%.

¹HNMR: (CDCl₃, 400 Hz) δ4.28˜4.22 (m, 1 H), 3.48˜3.44 (m, 2 H), 3.31˜3.28 (m, 2 H), 2.73 (s, 1 H), 2.6˜2.52 (m, 2 H), 2.15˜2.08 (m, 2 H), 1.57˜1.41 (m, 2 H), 1.37 (s, 9 H).

(2) A 25 mL single-necked bottle was taken and added with EH-006B (222 mg, 0.98 mmol), triethylamine (TEA, 222.2 mg, 2.2 mmol) and dichloromethane (5 mL) thereto, placed in an ice bath under nitrogen protection, and stirred and cooled to an internal temperature of about 4° C. Methanesulfonyl chloride (MsCl, 212.8 mg, 1.86 mmol) was added to the mixture with a syringe. After the addition, the reaction solution was warmed to room temperature and stirred for 1 hour, and water (5 mL) was added to stir, wash, and separate the liquid. The organic phase was dried with anhydrous sodium sulfate, and then the solvent was evaporated to dryness. Crude was subjected to column chromatography (PE:EA=3-1:1) to obtain a light yellow solid intermediate 2 (259 mg) with a yield rate of 86.9%.

¹HNMR: (CDCl₃, 400 Hz) δ5.12˜5.09 (m, 1 H), 3.61˜3.45 (m, 2 H), 3.43˜3.26 (m, 2 H), 2.99 (s, 3 H), 2.66˜2.65 (m, 2 H), 2.36˜2.29 (m, 2 H), 1.88˜1.82 (m, 2 H), 1.45 (s, 9 H).

Synthesis of Intermediate 3:

The structural formula of intermediate 3 is as follows:

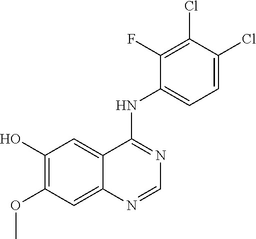 [SMILES: COc1cc2ncnc(Nc3ccc(Cl)c(Cl)c3F)c2cc1O]

The synthetic route is as follows:

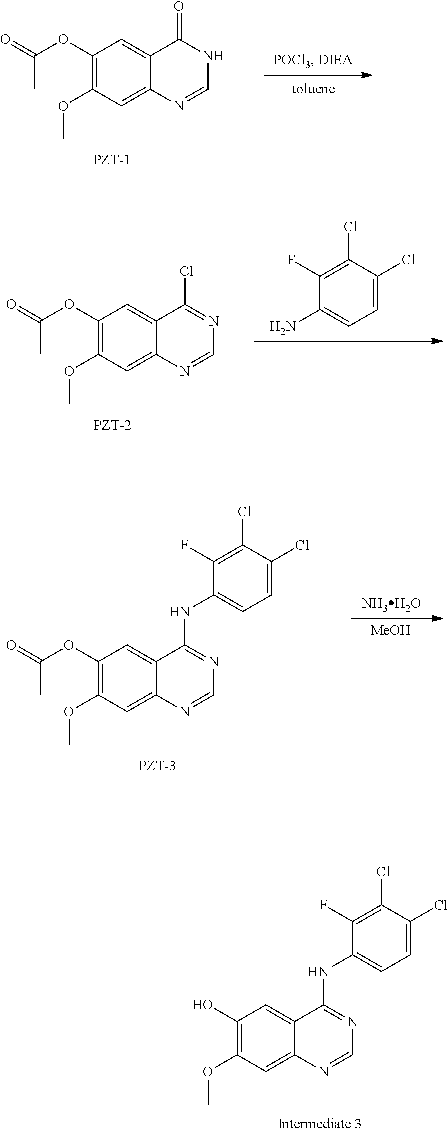

The experiment process is as follows:

(1) A 250 mL three-necked flask was taken and added with PZT-1 (10 g, 42.7 mmol), toluene (85 mL) and DIEA (6.46 g, 49.9 mmol) thereto, stirred well and added with phosphorus oxychloride (16.43 g, 107.2 mmol) thereto. The reaction solution was heated to an internal temperature of 75° C. under nitrogen protection, a large amount of white smoke was generated, the mixed solution was stirred and reacted at the same temperature for 3 hours, and the insolubles were gradually dissolved. A solution of 2-fluoro-3,4-dichloroaniline (8.45 g, 46.9 mmol) in toluene (45 mL) was added to the reaction solution at the same internal temperature. After the addition, the reaction solution was stirred and reacted at 75° C. for 3 hours, and a large amount of solid insolubles was gradually precipitated. The reaction solution was cooled to room temperature, an ice-water mixture (200 g) and ethyl acetate (200 mL) were added, and the pH value of the mixture was adjusted to about 8 with solid sodium bicarbonate under rapid stirring. There was a large amount of solid insoluble substance, which was filtered with suction, and the filter cake was washed with water and then with a small amount of ethyl acetate (30 mL). The filter cake was taken out and the solvent was dried to obtain a white powder PZT-3 (11.56 g) with a yield rate of 68.5%.

¹HNMR: (CDCl₃, 400 Hz) δ8.72 (s, 1 H), 8.48 (m, 1 H), 7.55 (s, 1 H), 7.33˜7.30 (m, 2 H), 3.96 (s, 3 H), 2.40 (s, 3 H).

(2) Synthesis of compound intermediate 3: A 250 mL single-neck flask was taken and added with PZT-3 (11.56 g, 29.2 mmol) and methanol (173 mL) thereto, then added with concentrated ammonia (53.2 g, 25%) at the room temperature. The reaction solution was stirred at room temperature and reacted and kept overnight, a large amount of white solid was precipitated, filtered through suction, and the filter cake was washed with methanol (20 mL). The filter cake was taken out and the solvent was evaporated to dryness to obtain a white powder intermediate 3 (8.9 g) with a yield rate of 86.4%.

¹HNMR: (DMSO-d6, 400 Hz) δ8.34 (s, 1 H), 7.65 (s, 1 H), 7.57-7.53 (m, 2 H), 7.33˜7.24 (m,3 H), 7.21 (s, 1 H), 3.97 (s, 3 H).

1. Specific examples of the compound used as a kinase inhibitor of the present disclosure

### EXAMPLE 1

The compound used as a kinase inhibitor in this example has a structural formula as follows:

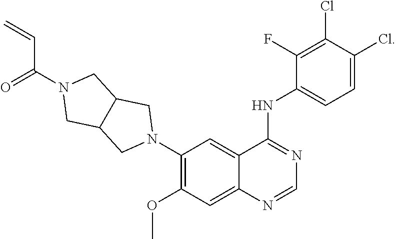 [SMILES: C=CC(=O)N1CC2CN(c3cc4c(Nc5ccc(Cl)c(Cl)c5F)ncnc4cc3OC)CC2C1]

The synthetic route is as follows:

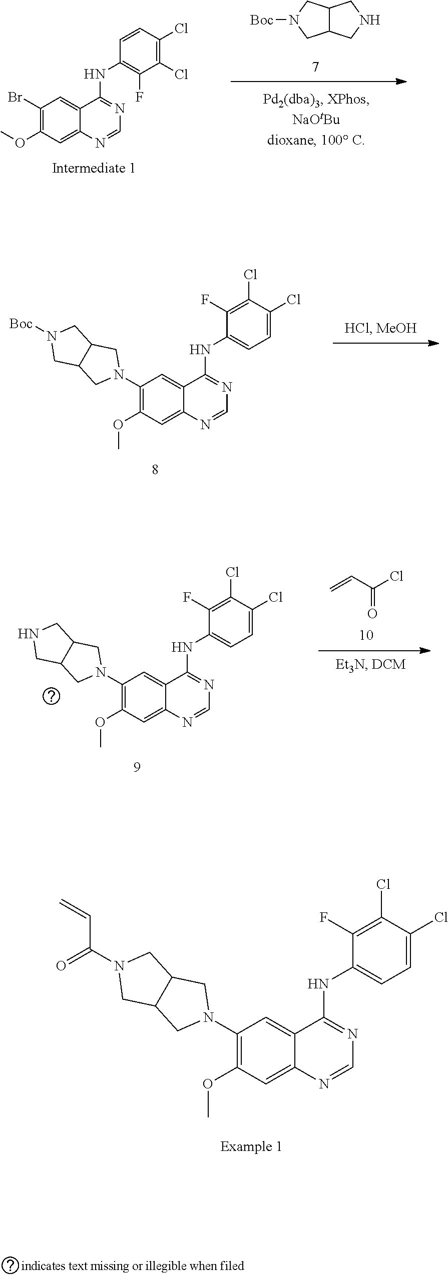

The specific synthesis process of the compound of the present embodiment is as follows:

(1) Synthesis of compound 8: In a dry 100 mL three-necked flask, the intermediate 1 (208 mg, 0.5 mmol), Pd2(dba)3 (tris(dibenzylideneacetone)dipalladium(0), 137 mg, 30 mol %), XPhos (2-dicyclohexylphosphorus-2′,4′,6′,-triisopropylbiphenyl, 143 mg, 60 mol %), NaOtBu (sodium tert-butoxide, 144 mg, 3.0 equiv), a compound 7 (212 mg, 1 mmol) and molecular sieve-dried dioxane (10 mL) were added thereto in sequence, reaction was carried out under argon at 100° C. for 16 hours. After the reaction was completed, the mixture was cooled to room temperature, a small amount of dichloromethane and an aqueous solution were added, filtered through celite, the mixed solution was separated into layers, and the aqueous phase was extracted with dichloromethane. Subsequently, the organic phase was washed with a saturated saline solution, dried and concentrated. Finally, the pure product was isolated by column chromatography: a yellow solid compound 8 (75 mg) with a yield rate of 27%.

¹HNMR: (CDCl₃, 400 Hz) δ: 8.61 (s, 1 H), 8.46 (t, J=8.6 Hz ,1 H), 7.38 (s, 1 H), 7.30 (dd, J₁=2.0 Hz, J₂=9.1 Hz, 1 H), 7.21 (s, 1 H), 6.73 (s, 1 H), 3.98 (s, 3 H), 3.64-3.72 (m, 4 H), 3.36-3.38 (m, 4 H), 3.00-3.03 (m, 2 H), 1.46 (s, 9 H).

(2) Synthesis of Compound of Example 1: A compound 8 (70 mg, 0.13 mmol) was added to a dry 25 mL single-neck flask, and 0.5 mL of methanol was added for dissolving, then HCl/MeOH solution (hydrochloric acid in methanol) was slowly added along the flask wall at room temperature, and stirred at room temperature for 2 hours under nitrogen until TLC showed that the compound 8 disappeared, and the reaction was stopped. The solution was directly spin-dried to obtain the crude product solid compound 9. Proceed to the next step with the yield rate of 90%. To the obtained solid compound 9, 5 mL of dichloromethane was added, and triethylamine (64.3 mg, 5.0 equiv) was added thereto, the temperature was lowered to 0° C., and acryloyl chloride 10 (11.5 mg, 1.0 equiv) diluted with DCM was slowly added dropwise, and the mixture was kept in an ice bath. The reaction was carried out under the bath for 0.5 to 1 hour until TLC showed the disappearance of the raw material. After the reaction was completed, an aqueous solution was added to quench, a small amount of dichloromethane was added, and the mixture was filtered through celite. The mixture was separated into layers and the aqueous phase was extracted with dichloromethane. Subsequently, the organic phase was washed with saturated brine solution, dried and concentrated. Finally, the pure product (10 mg) with a yield rate of 15% was obtained through preparative chromatography and separation.

¹HNMR: (CD₃OD, 400 Hz) δ: 8.58 (s, 1 H), 7.49-7.55 (m, 2 H), 7.40 (s, 1 H), 7.20 (s, 1 H), 6.63 (dd, J₁=10.3 Hz, J₂=16.8 Hz, 1 H), 6.28 (dd, J₁=1.9 Hz, J₂=16.8 Hz, 1 H), 5.75 (dd, J₁=1.9 Hz, J₂=10.5 Hz, 1 H), 4.08 (s, 3 H), 3.95-4.00 (m, 1 H), 3.77-3.87 (m, 3 H), 3.53-3.67 (m, 4 H), 3.09-3.22 (m, 2 H).

### EXAMPLE 2

The compound used as a kinase inhibitor in this example has the structural formula as follows:

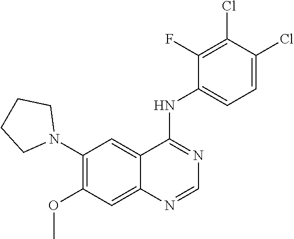 [SMILES: COc1cc2ncnc(Nc3ccc(Cl)c(Cl)c3F)c2cc1N1CCCC1]

The synthetic route is as follows:

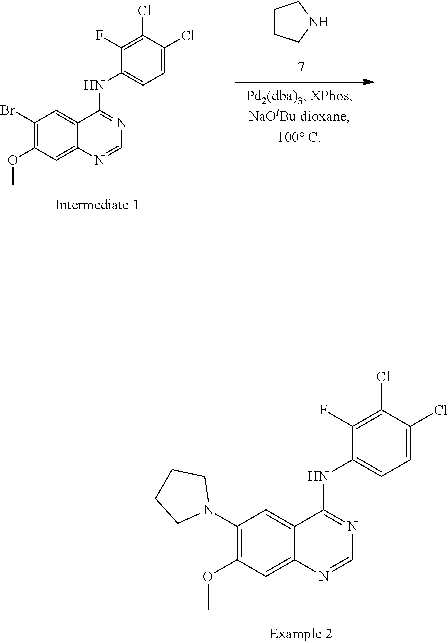 [SMILES: COc1cc2ncnc(Nc3ccc(Cl)c(Cl)c3F)c2cc1Br ; C1CCNC1 ; COc1cc2ncnc(Nc3ccc(Cl)c(Cl)c3F)c2cc1N1CCCC1]

The specific synthesis process is as follows:

In a dry 50 mL single-necked flask, intermediate 1 (107.7 mg, 0.25 mmol), Pd₂(dba)₃ (68.6 mg, 30 mol %), XPhos (71.5 mg, 60 mol %), NaOtBu (72 mg, 3.0 equiv), a compound 7 (53.3 mg, 0.75 mmol), and molecular sieve-dried dioxane (5 mL) were added thereto in sequence, reaction was carried out under argon at 100° C. for 16 hours. After the reaction was completed, the mixture was cooled to room temperature, a small amount of dichloromethane and an aqueous solution were added, filtered through celite, the mixture was separated into layers, and the aqueous phase was extracted with dichloromethane. Subsequently, the organic phase was washed with a saturated saline solution, dried and concentrated. Finally, the pure product (10 mg) with a yield rate of 10% was obtained through preparative chromatography and separation.

¹HNMR: (CDCl₃, 400 Hz) δ: 8.53 (s, 1 H), 7.49-7.55 (m, 2 H), 7.29 (d, J=4.1 Hz, 1 H), 7.14 (s, 1 H), 4.06 (s, 3 H), 3.59-3.62 (m, 4 H), 2.00-2.04 (m, 4 H).

### EXAMPLE 3

The compound used as a kinase inhibitor in this example has the structural formula as follows:

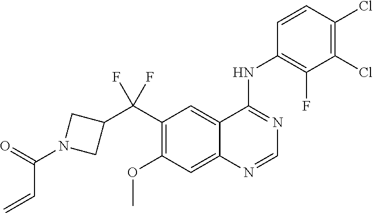 [SMILES: C=CC(=O)N1CC(C(F)(F)c2cc3c(Nc4ccc(Cl)c(Cl)c4F)ncnc3cc2OC)C1]

The synthetic route is as follows:

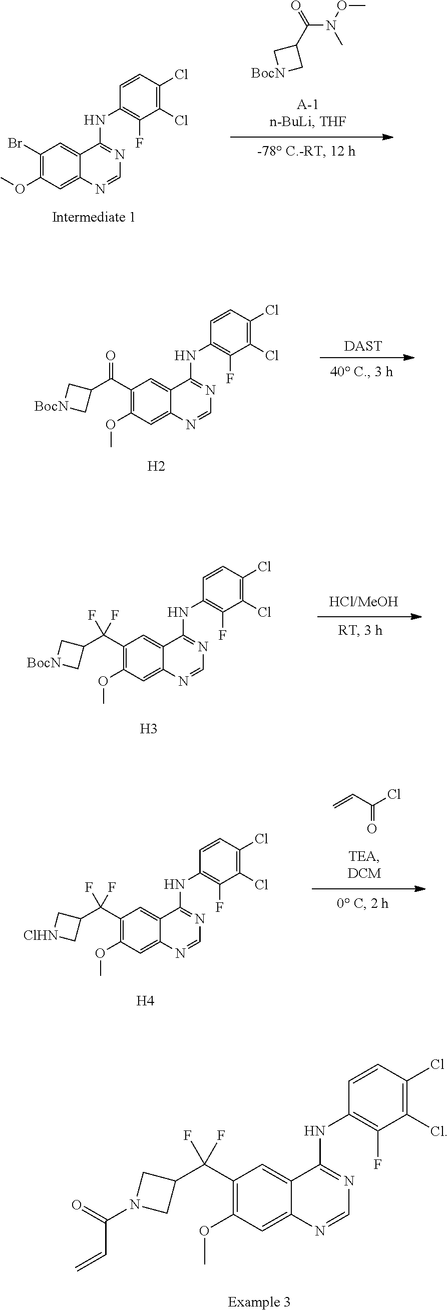

The experiment process is as follows:

(1) Synthesis of compound H2: In a 100 mL three-necked flask, a compound intermediate 1 (0.828 g, 2 mmol) and anhydrous THF (tetrahydrofuran, 30 mL) were added, then NaH (0.32 g, 6 mmol) was added, and then the temperature was lowered to −78 ° C.; n-butyllithium (0.96 mL, 2.4 mmol) was added under nitrogen protection, and the mixture was stirred at this temperature for 1 hour reaction. Next, a compound A-1 (0.584 g, 2.4 mmol) was dissolved in anhydrous THF (5 mL), and was slowly added to the reaction solution at −78° C.; after the dropwise addition was completed, the mixture was naturally raised to room temperature and stirred and kept overnight. Aqueous ammonium chloride solution (30 mL) was added to the reaction solution, extracted three times with ethyl acetate (30 mL), the organic phases were combined, dried, and purified through column chromatography (PE:EA=1:1) to obtain 0.3 g of the product with a yield rate of 27%.

¹HNMR: (CDCl₃, 400 Hz) δ8.74 (s, 1 H), 8.48 (s, 1 H), 7.38 (t, J=7.6 Hz, 1 H), 7.36˜7.30(m, 2 H), 4.20˜4.15 (m, 5 H), 4.05 (s, 3 H), 1.44 (s, 9 H).

(2) Synthesis of compound H3: A compound H2 (0.3 g, 0.57 mmol), DAST (diethylaminosulfur trifluoride, 10 mL), DCM (dichloromethane, 5 mL) were added to a 20 mL single-necked flask, and the temperature was raised to 40° C. under nitrogen protection. The mixture was stirred for reaction for 3 hours. Then the reaction solution was carefully added to the aqueous sodium bicarbonate solution, and then extracted with ethyl acetate three times. The organic phases were combined, and column chromatography (PE:EA=1:1) was performed to obtain 90 mg of the product with a yield rate of 28%.

(3) Synthesis of compound H4: A compound H3 (70 mg, 0.13 mmol) was dissolved in 4 N HCl/MeOH, stirred at room temperature for 3 hours, and then spin-dried for the next step.

(4) Synthesis of Compound of Example 3: A compound H4 (65 mg, 0.13 mmol), TEA (triethylamine, 80 mg, 0.78 mmol) and DCM (10 mL) were added to a 20 mL single-necked flask, the temperature was lowered to 0° C. under nitrogen protection, a solution of acryloyl chloride (12 mg, 0.13 mmol) in DCM (2 mL) was added, and the reaction was carried out at this temperature for 0.5 hour. Aqueous sodium bicarbonate solution (20 mL) was added, extracted with DCM for three times, and the organic phases were combined, dried, and spin-dried to prepare and separate to obtain 10 mg of the product with a yield rate of 15%.

¹HNMR: (CD₃OD, 400 Hz) δ8.82 (s, 1 H) , 8.75 (s, 1 H), 8.57˜8.51 (m, 2 H), 7.36 (s, 1 H), 6.39˜6.24 (m, 2 H), 5.77˜5.74 (m, 1 H), 4.43˜4.40 (m, 2 H), 4.12˜4.09 (m, 5 H), 3.97˜3.90 (m, 1 H).

### EXAMPLE 4

The compound of the present embodiment used as a kinase inhibitor has the structural formula as follows:

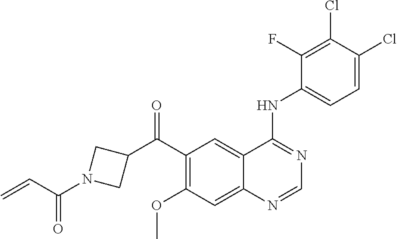 [SMILES: C=CC(=O)N1CC(C(=O)c2cc3c(Nc4ccc(Cl)c(Cl)c4F)ncnc3cc2OC)C1]

The synthetic route is as follows:

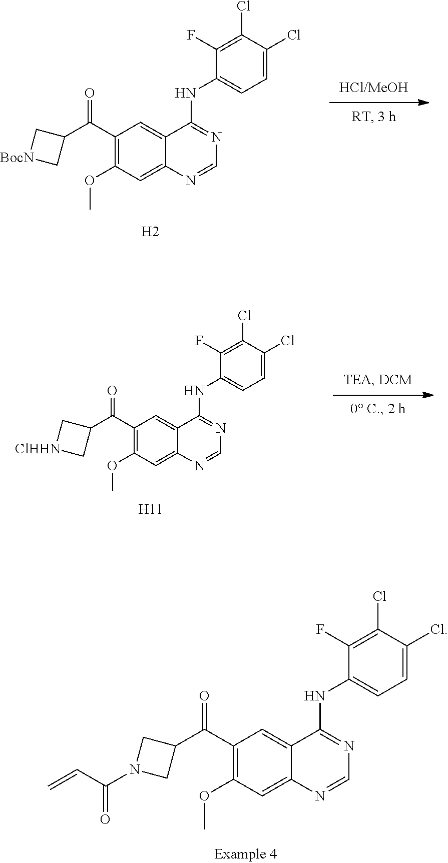

The experiment process is as follows:

(1) Synthesis of compound H11: A compound H2 (52 mg, 0.1 mmol) was dissolved in 4 N HCl/MeOH, stirred at room temperature for 3 hours, and then spin-dried for the next step.

(2) Synthesis of Compound of Example 4: A compound H11 (52 mg, 0.1 mmol), TEA (60 mg, 0.6 mmol) and DCM (10 mL) were added to a 20 mL single-necked flask, and the temperature was lowered to 0° C. under nitrogen protection, added with acryloyl chloride (9 mg, 0.1 mmol) in DCM (2 mL), reacted at this temperature for 0.5 hour, added with aqueous sodium bicarbonate (20 mL), and extracted with DCM for three times. The organic phases were combined, dried, spin-dried, and 15 mg of product with a yield rate of 21% was obtained through preparative chromatographic and separation.

¹HNMR: (CD₃OD, 400 Hz) δ9.02 (s, 1 H), 8.75 (s, 1 H), 8.57˜8.51 (m, 2 H), 7.38 (s, 1 H), 6.39˜6.27 (m, 2 H), 5.77˜5.74 (m, 1 H), 4.54˜4.50 (m, 2 H), 4.35˜4.30 (m, 3 H), 4.18 (s, 3 H).

### EXAMPLE 5

The compound used as a kinase inhibitor in this example has the structural formula as follows:

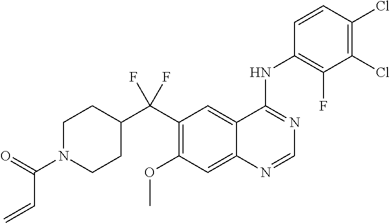 [SMILES: C=CC(=O)N1CCC(C(F)(F)c2cc3c(Nc4ccc(Cl)c(Cl)c4F)ncnc3cc2OC)CC1]

The synthetic route is as follows:

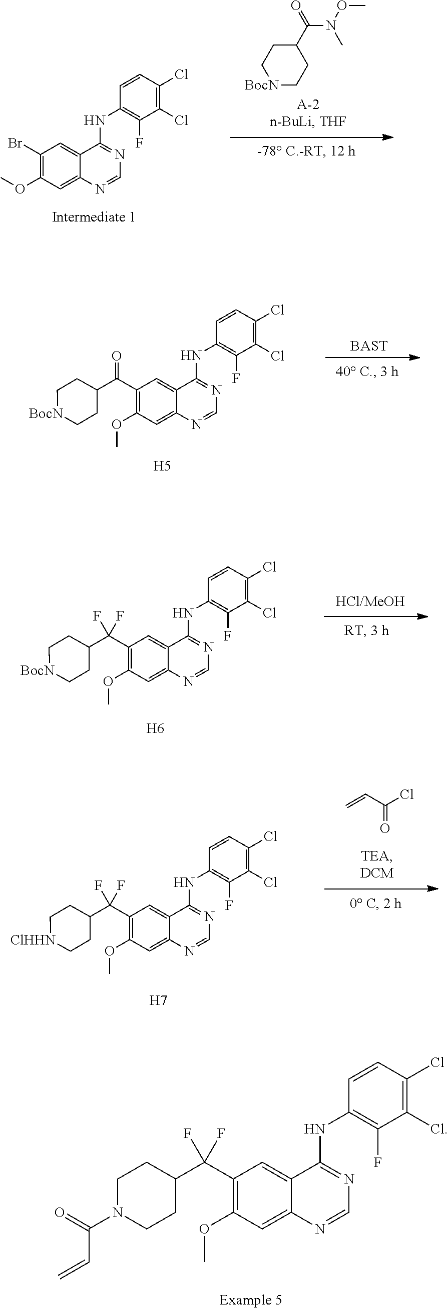

The experiment process is as follows:

(1) Synthesis of compound H5: In a 100 mL three-necked flask, a compound intermediate 1 (1.242 g, 3 mmol) and anhydrous THF (30 mL) were added thereto, then added with NaH (0.48 g, 9 mmol), cooled to −78° C., and added with n-butyllithium (1.44 mL, 3.6 mmol) under nitrogen protection; the mixture was stirred at this temperature for reaction for 1 hour, after which a compound A-2 (0.978 g, 3.6 mmol) was dissolved in anhydrous THF (5 mL), and was slowly added to the reaction solution at −78° C. After the dropwise addition was completed, the mixture was naturally raised to room temperature and stirred and kept overnight. Aqueous ammonium chloride solution (30 mL) was added to the reaction solution, and extracted for three times with ethyl acetate (30 mL). The organic phases were combined, dried, and purified by column chromatography (PE:EA=1:1) to obtain 0.51 g of product with a yield rate of 23%.

¹HNMR: (CDCl₃, 400 Hz) δ8.75 (s, 1 H), 8.44 (t, J=8.4 Hz, 1 H), 8.11 (s, 1 H), 7.58 (s, 1 H), 7.35˜7.32 (m, 2 H), 4.13˜4.06 (m, 5 H), 3.47˜3.40 (m, 1 H), 2.90˜2.83 (m, 2 H), 1.88˜1.83 (m, 2 H), 1.65˜1.60 (m, 2 H), 1.46 (s, 9 H).

(2) Synthesis of compound H6: A compound H5 (0.5 g, 0.91 mmol), BAST (5 mL), DCM (5 mL) were added to a 20 mL single-necked flask, and the temperature was raised to 45° C. under nitrogen protection, stirred and reacted for 4.5 hours. The reaction solution was carefully added to aqueous sodium bicarbonate solution, then extracted with ethyl acetate for three times. The organic phases were combined, and 50 mg of the product with a yield rate of 10% was obtained through column chromatography (PE:EA=1:1).

(3) Synthesis of compound H7: A compound H6 (50 mg, 0.1 mmol) was dissolved in 4 N HCl/MeOH, stirred at room temperature for 3 hours, and then spin-dried to proceed to the next step.

(4) Synthesis of Compound of Example 5: A compound H7 (45 mg, 0.1 mmol), TEA (60 mg, 0.6 mmol) and DCM (10 mL) were added to a 20 mL single-neck flask, and the temperature was lowered to 0° C. under nitrogen protection, added with acryloyl chloride (9 mg, 0.1 mmol) in DCM (2 mL), and reacted at this temperature for 0.5 hour, added with aqueous sodium bicarbonate (20 mL), and extracted for three times with DCM. The organic phases were combined, dried, and spin-dried, and 8 mg of the product with a yield rate of 12% was obtained through preparative chromatography and separation.

¹HNMR: (CD₃OD, 400 Hz) δ8.75 (s, 1 H), 8.70 (s, 1 H), 8.53˜8.50 (m, 2 H), 7.36 (s, 1 H), 6.79˜6.73 (m, 1 H), 6.21˜6.16 (m, 1 H), 5.75˜5.72 (m, 1 H), 4.23˜4.18 (m, 1 H), 4.12˜4.09 (m, 1 H), 3.34 (s, 3 H), 3.17˜2.89 (m, 3 H), 1.83˜1.68 (m, 2 H), 1.55˜1.46 (m, 2 H).

### EXAMPLE 6

The compound used as a kinase inhibitor in this example has the structural formula as follows:

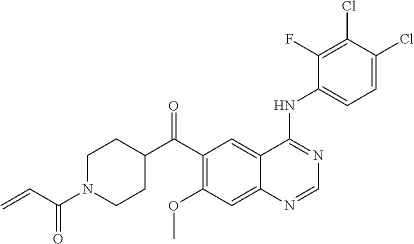 [SMILES: C=CC(=O)N1CCC(C(=O)c2cc3c(Nc4ccc(Cl)c(Cl)c4F)ncnc3cc2OC)CC1]

The synthetic route is as follows:

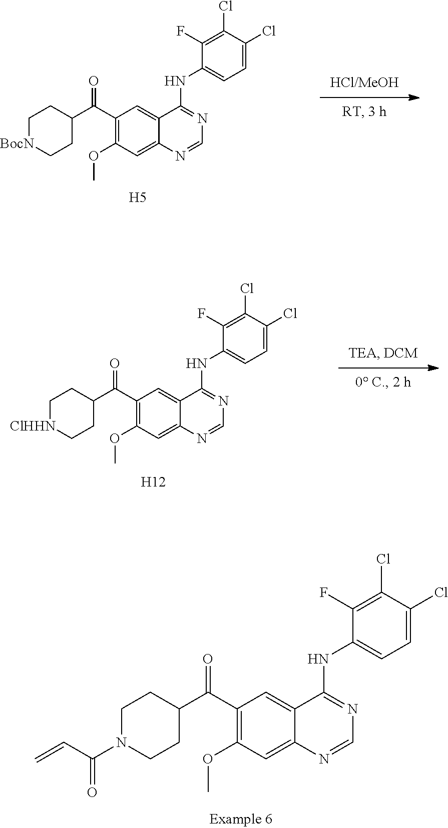

The experiment process is as follows:

(1) Synthesis of compound H12: A compound H5 (55 mg, 0.1 mmol) was dissolved in 4 N HCl/MeOH, stirred at room temperature for 3 hours, and then spin-dried for the next step.

(2) Synthesis of Compound of Example 6: A compound H12 (52 mg, 0.1 mmol), TEA (60 mg, 0.6 mmol) and DCM (10 mL) were added to a 20 mL single-neck flask, and the temperature was lowered to 0° C. under nitrogen protection, added with acryloyl chloride (9 mg, 0.1 mmol) in DCM (2 mL), reacted at this temperature for 0.5 hour, added with aqueous sodium bicarbonate (20 mL), and extracted for three times with DCM. The organic phases were combined, dried, spin-dried, and 10 mg of product with a yield rate of 20% was obtained through preparative chromatographic and separation.

¹HNMR: (CD₃OD, 400 Hz) δ8.77 (s, 1H), 8.68 (s, 1 H), 7.55˜7.50 (m, 2 H), 7.37 (s, 1 H), 6.81˜6.74 (m, 1 H), 6.21˜6.17 (m, 1 H), 6.75˜6.72 (m, 1 H), 4.48˜4.45 (m, 2 H), 4.16˜4.11 (m, 5 H), 3.63˜3.57 (m, 1 H), 3.57˜3.50 (m, 1 H), 3.05˜2.94 (m,1 H), 2.01˜195 (m, 2 H), 1.67˜1.60 (m, 2 H).

### EXAMPLE 7

The compound used as a kinase inhibitor in this example has the structural formula as follows:

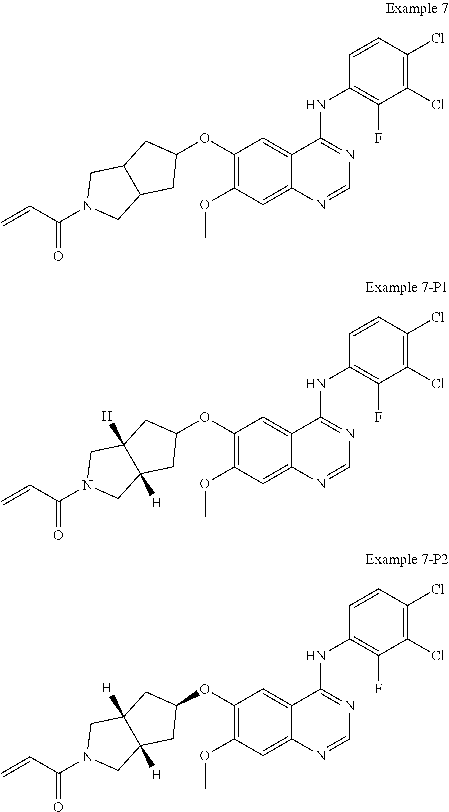 [SMILES: C=CC(=O)N1CC2CC(Oc3cc4c(Nc5ccc(Cl)c(Cl)c5F)ncnc4cc3OC)CC2C1 ; C=CC(=O)N1C[C@H]2CC(Oc3cc4c(Nc5ccc(Cl)c(Cl)c5F)ncnc4cc3OC)C[C@H]2C1 ; C=CC(=O)N1C[C@H]2C[C@H](Oc3cc4c(Nc5ccc(Cl)c(Cl)c5F)ncnc4cc3OC)C[C@H]2C1]

The synthetic route is as follows:

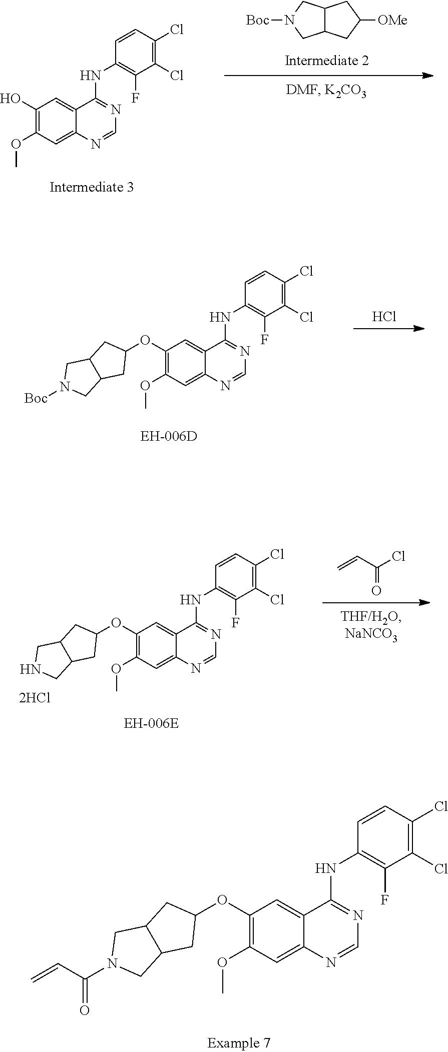

The experiment process is as follows:

(1) Synthesis of compound EH-006D: A 25 mL single-necked bottle was taken and added with an intermediate 3 (273.9 mg, 0.77 mmol) and an intermediate 2 (236 mg, 0.77 mmol) thereto, and then added with anhydrous potassium carbonate (320.7 mg, 2.32 mmol) and DMF (11 mL). The mixture was placed in an oil bath at 85° C. for heating, stirring and reacting overnight, the solvent was evaporated to dryness under reduced pressure, water (70 mL) and ethyl acetate (50 mL) were added to the residue. The mixture was stirred and separated to collect the organic phase, and the aqueous phase was extracted once with ethyl acetate (50 mL). The organic phases were combined, dried over anhydrous sodium sulfate, and the solvent was evaporated under reduced pressure. The crude product was subjected to column chromatography (PE:EA=1:1) to obtain a white solid EH-006D (169 mg) with a yield rate of 38.9%.

(2) Synthesis of compound EH-006E: A 50mL single-necked bottle was taken and added with EH-006D (169 mg, 0.30 mmol) and methanol (6 mL) thereto, but the mixture could not be completely dissolved, then added with concentrated hydrochloric acid (6mL, 37%) at room temperature, so that the solid was completely dissolved to obtain a yellow solution. The yellow solution was stirred at room temperature for 2 hours and the solvent was evaporated to dryness to obtain a yellow powder EH-006E (160 mg) with a yield rate of 99.3%.

(3) Synthesis of Compound of Example 7: A 50 mL three-necked flask was taken and added with EH-006E (160 mg, 0.30 mmol), sodium bicarbonate (150.7 mg, 1.79 mmol), THF (4 mL) and pure water (4 mL). The reaction solution was cooled in an ice bath to about 4° C. under nitrogen protection. Under this condition, a solution of acryloyl chloride (40.6 mg, 0.45 mmol) in THF (3 mL) was added to the reaction solution with a syringe, and the ice bath was removed after the addition. The reaction solution was stirred at room temperature, reacted and kept overnight, the pH value of the mixture was adjusted to about 8 with solid sodium bicarbonate, and ethyl acetate (50 mL) and water (50 mL) were added thereto. The organic phase was collected by separation, and the aqueous phase was extracted with ethyl acetate (40 mL). The combined organic phases were dried over anhydrous sodium sulfate, evaporated to dryness under reduced pressure, and the residue was subjected to column chromatography (ethyl acetate as the mobile phase) to obtain a white solid compound Example 7 (53 mg), which was purified through preparative chromatography to obtain 15.7 mg of Example 7-P1 and Example 7-P2 in total with a yield rate of 10.2%.

¹HNMR: (CDCl₃, 400 Hz) δ11.23 (s, 1 H), 8.40 (s, 1 H), 1.88 (s, 1 H), 7.35˜7.30 (m, 2 H), 7.25˜7.21 (m, 1 H), 6.43˜6.37 (m, 1 H), 6.31˜6.27 (m, 1 H), 5.68˜5.66 (d, J=8 Hz, 1 H), 5.12 (s, 1 H), 3.98 (s, 3 H), 3.79˜3.70 (m, 2 H), 3.43˜3.41 (m, 2 H), 3.05˜2.94 (m, 2 H), 2.29˜2.21 (m, 2 H), 2.04˜1.98 (m, 2 H).

### EXAMPLES 8-22

The compounds of Examples 8-22 used as kinase inhibitors have the structural formulae as listed in Table 1 below, respectively.

TABLE 1
Compounds of Examples 8-22 Used as Kinase Inhibitors

| No. of | | | Characterization |
|---|---|---|---|
| Example | Structural formulae | Preparation process | results |
| 8 | 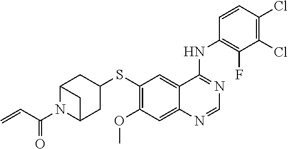 [SMILES: C=CC(=O)N1C2CC(Sc3cc4c(Nc5ccc(Cl)c(Cl)c5F)ncnc4cc3OC)CC1C2] | With reference to Example 7, the intermediate 3 in the first step was replaced with | [M + H]⁺ 519.1/521.1 |
| | | 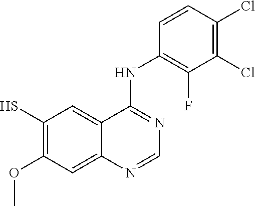 [SMILES: COc1cc2ncnc(Nc3ccc(Cl)c(Cl)c3F)c2cc1S] | |
| 9 | 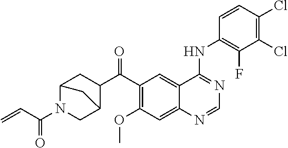 [SMILES: C=CC(=O)N1CC2CC1CC2C(=O)c1cc2c(Nc3ccc(Cl)c(Cl)c3F)ncnc2cc1OC] | With reference to Example 6, H5 in the first step was replaced with | [M + H]⁺ 515.1/517.1 |
| | | 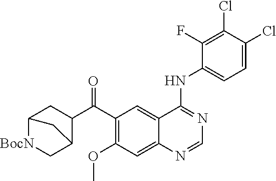 [SMILES: COc1cc2ncnc(Nc3ccc(Cl)c(Cl)c3F)c2cc1C(=O)C1CC2CC1CN2C(=O)OC(C)(C)C] | |
| 10 | 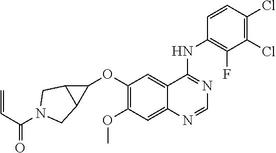 [SMILES: C=CC(=O)N1CC2C(C1)C2Oc1cc2c(Nc3ccc(Cl)c(Cl)c3F)ncnc2cc1OC] | Referring to Example 1, the raw material 7 in the first step was replaced with | [M + H]⁺ 489.1/491.1 |
| | | 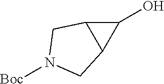 [SMILES: CC(C)(C)OC(=O)N1CC2C(O)C2C1] | |
| 11 | 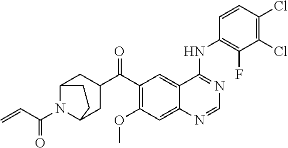 [SMILES: C=CC(=O)N1C2CCC1CC(C(=O)c1cc3c(Nc4ccc(Cl)c(Cl)c4F)ncnc3cc1OC)C2] | With reference to Example 6, H5 in the first step was replaced with | [M + H]⁺ 529.1/531.1 |
| | | 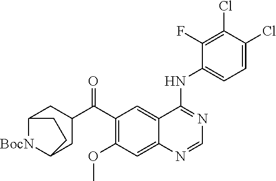 [SMILES: COc1cc2ncnc(Nc3ccc(Cl)c(Cl)c3F)c2cc1C(=O)C1CC2CCC(C1)N2C(=O)OC(C)(C)C] | |
| 12 | 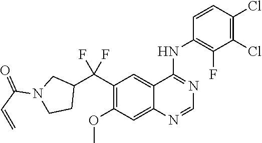 [SMILES: C=CC(=O)N1CCC(C(F)(F)c2cc3c(Nc4ccc(Cl)c(Cl)c4F)ncnc3cc2OC)C1] | With reference to Example 5, A-2 in the first step was replaced with | [M + H]⁺ 511.1/513.1 |
| | | 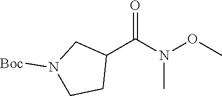 [SMILES: CON(C)C(=O)C1CCN(C(=O)OC(C)(C)C)C1] | |
| 13 | 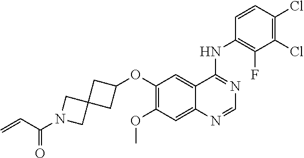 [SMILES: C=CC(=O)N1CC2(CC(Oc3cc4c(Nc5ccc(Cl)c(Cl)c5F)ncnc4cc3OC)C2)C1] | With reference to Example 7, the intermediate 2 in the first step was replaced with | [M + H]⁺ 503.1/505.1 |
| | | 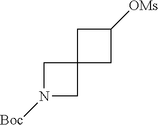 [SMILES: CC(C)(C)OC(=O)N1CC2(CC(OS(C)(=O)=O)C2)C1] | |
| 14 | 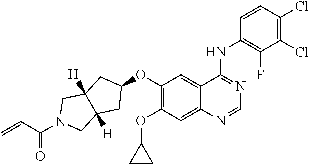 [SMILES: C=CC(=O)N1C[C@H]2C[C@H](Oc3cc4c(Nc5ccc(Cl)c(Cl)c5F)ncnc4cc3OC3CC3)C[C@H]2C1] | With reference to Example 7, the intermediate 3 in the first step was replaced with | [M + H]⁺ 543.1/545.1 |
| | | 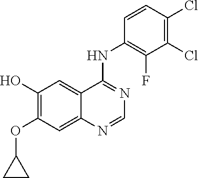 [SMILES: Oc1cc2c(Nc3ccc(Cl)c(Cl)c3F)ncnc2cc1OC1CC1] | |
| 15 | 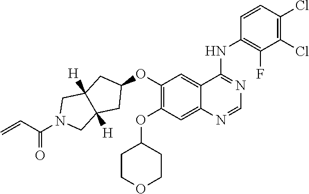 [SMILES: C=CC(=O)N1C[C@H]2C[C@H](Oc3cc4c(Nc5ccc(Cl)c(Cl)c5F)ncnc4cc3OC3CCOCC3)C[C@H]2C1] | With reference to Example 7, the intermediate 3 in the first step was replaced with | [M + H]⁺ 587.2/589.2 |
| | | 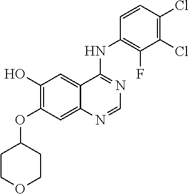 [SMILES: Oc1cc2c(Nc3ccc(Cl)c(Cl)c3F)ncnc2cc1OC1CCOCC1] | |
| 16 | 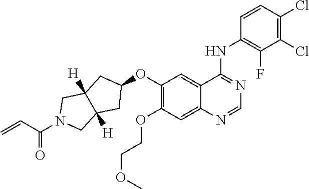 [SMILES: C=CC(=O)N1C[C@H]2C[C@H](Oc3cc4c(Nc5ccc(Cl)c(Cl)c5F)ncnc4cc3OCCOC)C[C@H]2C1] | With reference to Example 7, the intermediate 3 in the first step was replaced with | [M + H]⁺ 561.1.1/563.1 |
| | | 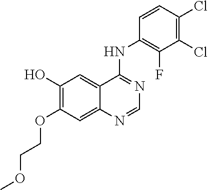 [SMILES: COCCOc1cc2ncnc(Nc3ccc(Cl)c(Cl)c3F)c2cc1O] | |
| 17 | 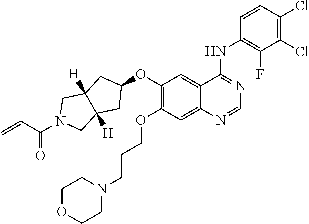 [SMILES: C=CC(=O)N1C[C@H]2C[C@H](Oc3cc4c(Nc5ccc(Cl)c(Cl)c5F)ncnc4cc3OCCCN3CCOCC3)C[C@H]2C1] | With reference to Example 7, the intermediate 3 in the first step was replaced with | [M + H]⁺ 630.2/632.2 |
| | | 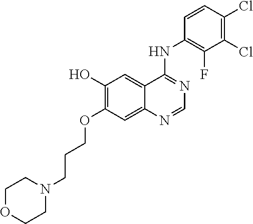 [SMILES: Oc1cc2c(Nc3ccc(Cl)c(Cl)c3F)ncnc2cc1OCCCN1CCOCC1] | |
| 18 | 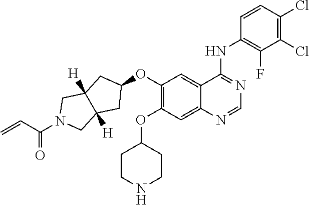 [SMILES: C=CC(=O)N1C[C@H]2C[C@H](Oc3cc4c(Nc5ccc(Cl)c(Cl)c5F)ncnc4cc3OC3CCNCC3)C[C@H]2C1] | With reference to Example 7, the intermediate 3 in the first step was replaced with | [M + H]⁺ 586.2/588.2 |
| | | 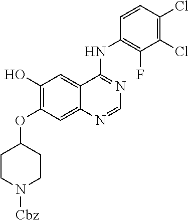 [SMILES: O=C(OCc1ccccc1)N1CCC(Oc2cc3ncnc(Nc4ccc(Cl)c(Cl)c4F)c3cc2O)CC1] | |
| 19 | 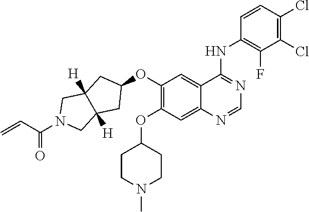 [SMILES: C=CC(=O)N1C[C@H]2C[C@H](Oc3cc4c(Nc5ccc(Cl)c(Cl)c5F)ncnc4cc3OC3CCN(C)CC3)C[C@H]2C1] | With reference to Example 7, the intermediate 3 in the first step was replaced with | [M + H]⁺ 600.2/602.2 |
| | | 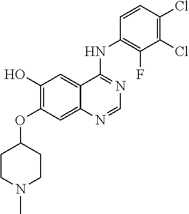 [SMILES: CN1CCC(Oc2cc3ncnc(Nc4ccc(Cl)c(Cl)c4F)c3cc2O)CC1] | |
| 20 | 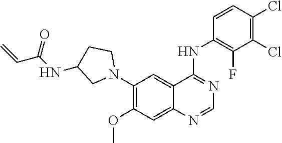 [SMILES: C=CC(=O)NC1CCN(c2cc3c(Nc4ccc(Cl)c(Cl)c4F)ncnc3cc2OC)C1] | Referring to Example 1, the raw material 7 in the first step was replaced with | [M + H]⁺ 476.1/478.1 |
| | | 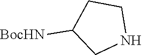 [SMILES: CC(C)(C)OC(=O)NC1CCNC1] | |
| 21 | 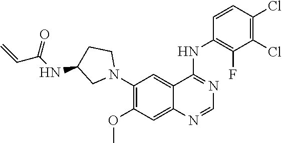 [SMILES: C=CC(=O)N[C@H]1CCN(c2cc3c(Nc4ccc(Cl)c(Cl)c4F)ncnc3cc2OC)C1] | Example 20 was obtained through chiral preparative chromatography and separation | [M + H]⁺ 476.1/478.1 |
| 22 | 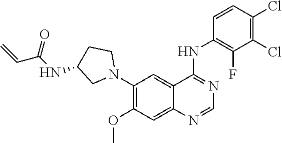 [SMILES: C=CC(=O)N[C@@H]1CCN(c2cc3c(Nc4ccc(Cl)c(Cl)c4F)ncnc3cc2OC)C1] | Example 20 was obtained through chiral preparative chromatography and separation | [M + H]⁺ 476.1/478.1 |

2. In Application Example, the following biological activity test is used to describe the inhibitory activity of the compounds involved in the present disclosure on EGFR exon 20 insertion mutation.

The kinase activity assay was utilized to screen the activity of the compounds prepared in the example on the EGFR exon 20 insertion mutant kinase at the concentration of ATP Km, and staurosporine was adopted as a reference substance. Screening of biological activity of compounds will be performed in duplicate at 10 concentrations.

1. Test sample

Each sample was prepared as a solution with a concentration of 10 mM.

2. Experimental Method

1) Basic buffer solution and quench buffer solution for experimental kinases were prepared.

20 mMHepes (pH 7.5), 10 mM MgCl2, 1 mM EGTA, 0.02% Brij35, 0.02 mg/ml BSA, 0.1 mM Na3VO4, 2 mM DTT, 1% DMSO.

2) Compounds for experimental kinases were prepared.

Compounds for test were dissolved to specific concentrations in 100% dimethyl sulfoxide. (Serial) dilutions were performed with Integra Viaflo Assist assisted DMSO.

3) Reaction Steps

The kinase was added to freshly prepared basic reaction buffer, and any desired cofactors were added to the above substrate solution.

The EGFR exon 20 insertion mutant kinase was added to the substrate solution and mixed gently; the compound in 100% dimethyl sulfoxide was fed into the kinase reaction mixture by using Acoustic technology (Echo550; nanoliter range) and incubated for 20 minutes at room temperature.

33P-ATP (Specific activity 10 Ci/l) was added to the reaction mixture to start the reaction, incubated at room temperature for 2 hours, and the radioactivity was detected by the filter-binding method.

Kinase activity data are expressed as a percentage of remaining kinase activity in the test sample compared to the medium (dimethyl sulfoxide) reaction. IC₅₀ values and curve fitting were obtained by using Prism (GRAPHPAD software).

The test results of the inhibitory activity (IC₅₀ (nM) value) of the obtained test samples against EGFR exon 20 and Her2 exon20 insertion mutant kinases are shown in Table 2 and Table 3. In Table 2, A<4.0 nM, 4.0 nM≤B<40 nM, C≥40 nM; ND: not detected.

TABLE 2
Inhibitory activity of the compounds of the present disclosure
on EGFR exon20 and Her2 exon20 insertion mutant kinases

| | EGFR | | Her2 |
|---|---|---|---|
| | | D770- | A775- |
| | A763_Y764insFHEA | N771insNPG | G776insYVMA |
| Compound | IC50 (nM) | IC50 (nM) | IC50 (nM) |
| Staurosporine | C | C | C |
| Poziotinib | B | A | B |
| Example 1 | B | A | ND |
| Example 2 | A | A | ND |
| Example 3 | A | A | A |
| Example 4 | B | A | ND |
| Example 5 | A | A | A |
| Example 6 | B | B | ND |
| Example 7 | B | A | A |
| Example 8 | B | B | ND |
| Example 9 | B | B | B |
| Example 10 | A | A | ND |
| Example 11 | B | B | B |
| Example 13 | B | A | A |
| Example 16 | A | A | A |
| Example 17 | B | A | A |

The specific IC₅₀ values of some compounds of the present disclosure are shown in Table 3 below.

TABLE 3
Inhibitory activity of different compounds on EGFR
exon 20 and Her2 exon20 insertion mutant kinases

| | EGFR | | Her2 |
|---|---|---|---|
| | | D770- | A775- |
| | A763_Y764insFHEA | N771insNPG | G776insYVMA |
| Compound | IC50 (nM) | IC50 (nM) | IC50 (nM) |
| Staurosporine | 160.4 | 46.9 | 80.7 |
| Poziotinib | 4.27 | 0.943 | 4.79 |
| Example 1 | 6.68 | 1.61 | ND |
| Example 2 | 3.03 | 1.65 | ND |
| Example 3 | 1.32 | 0.513 | 2.61 |
| Example 4 | 11.8 | 3.04 | ND |
| Example 5 | 2.19 | 1.54 | 1.02 |
| Example 6 | 16.2 | 4.51 | ND |
| Example 7 | 4.04 | 0.833 | 1.84 |

(ND: not detected)

As can be seen from the above table, through the in vitro biological activity screening and with staurosporine as the control group, the compounds we synthesized have good inhibitory ability on EGFR exon 20 insertion mutant kinase, most of which have the same inhibitory activity as poziotinib. Specifically, the activity of the compound of Example 3 was 2-3 times that of poziotinib. The inhibitory activity of the compound of Example 5 on Her2 insertion mutant kinase was about 4 times that of poziotinib, and therefore the compound is highly expected to be further developed as a drug for modulating the activity of EGFR exon 20 insertion mutant kinase or treating diseases related to EGFR exon 20 insertion mutant kinase. Specifically, in the preparation of medicines, the medicines may be prepared in conventional forms such as capsules or tablets.

1. A compound used as a kinase inhibitor, wherein the compound is represented by formula I, or an isomer thereof, or a pharmaceutically acceptable salt, solvate or prodrug thereof; 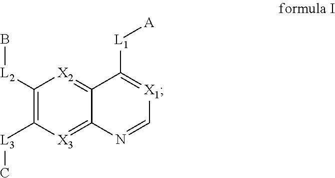
wherein in formula I, X₁ is selected from N or CR₂; X₂ is selected from N or CR₃; X₃ is selected from N or CR₄;
L₁ and L₃ are each independently selected from a single bond, 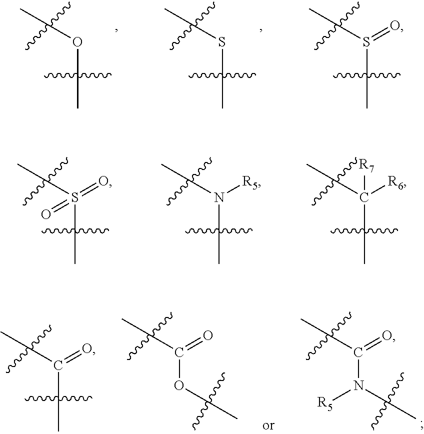
L₂ is selected from a single bond, 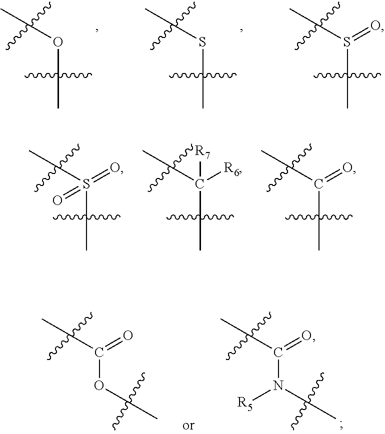
A is selected from C₆₋₁₀ aryl, and C₅₋₁₂ heteroaryl; or A is C₆₋₁₀ aryl substituted with 1, 2 or 3 substituents or C₅₋₁₂ heteroaryl substituted with 1, 2 or 3 substituents; the substituent is selected from any one of H, halogen, cyano, amino, ester, urea, carbamate, amide, C₁₋₆ alkyl, C₁₋₆ alkoxy, C₃₋₆ cycloalkyl, C₃₋₆ cycloalkoxy, C₆₋₁₀ aryl, and C₅₋₁₂ heteroaryl; or the substituent is amino group, ester group, urea group, carbamate group, amide group, C₁₋₆ alkyl group, C₁₋₆ alkoxy group, C₃₋₆ cycloalkyl group, C₃₋₆ cycloalkoxy group, C₆₋₁₀ aryl, and C₅₋₁₂ heteroaryl, which is substituted with 1, 2 or 3 R;
R is selected from halogen, cyano, hydroxyl, amino, ester, urea, carbamate, amide, C₁₋₆ alkyl, C₁₋₆ alkoxy, C₃₋₆ cycloalkyl, C₃₋₆ cycloalkoxy, C₂₋₆ alkenyl, C₂₋₆ alkynyl, C₆₋₁₀ aryl, and C₅₋₁₂ heteroaryl;
B is a nitrogen-containing heterocyclic group or a nitrogen-containing heterocyclic group substituted by R₁, and the number of nitrogen heteroatoms in the nitrogen-containing heterocyclic group is one or more;
the R₁ is selected from 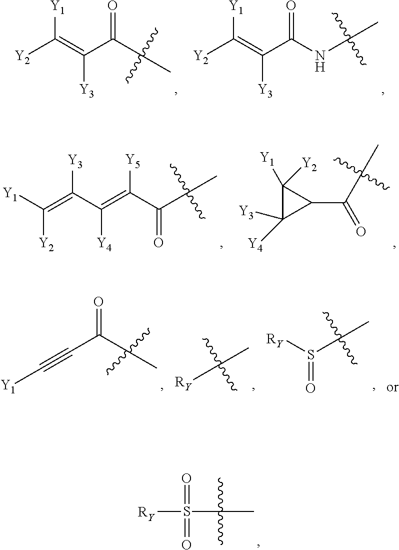
and Y₁, Y₂, Y₃, Y₄, and Y₅ are each independently selected from hydrogen, halogen, C₁₋₁₂ alkyl, C₃₋₁₂ cycloalkyl, and C₁₋₁₂ alkylamino; or Y₁, Y₂, Y₃, Y₄, and Y₅ are each independently C₁₋₁₂ alkyl, C₃₋₁₂ cycloalkyl, or C₁₋₁₂ alkylamino, which is substituted by the R; R_{Y} is selected from C₁₋₁₂ alkyl, C₁₋₁₂ alkyl substituted by the R, C₃₋₁₂ cycloalkyl, and C₃₋₁₂ cycloalkyl substituted by the R; or R_{Y} is C₁₋₁₂ alkyl, C₁₋₁₂ alkyl substituted by the R, C₃₋₁₂ cycloalkyl group, or a group formed by replacing one or more carbon atoms in the C₃₋₁₂ cycloalkyl group substituted by the R with one or more heteroatoms in N, O, and S;
wherein R₂, R₃, R₄, R₅, R₆ and R₇ are each independently selected from H, halogen, cyano, amino, ester, urea, carbamate, amide, C₁₋₆ alkyl, C₁₋₆ alkoxy, C₃₋₆ cycloalkyl, C₃₋₆ cycloalkoxy, C₆₋₁₀ aryl, and C₅₋₁₂ heteroaryl; or R₂, R₃, R₄, R₅, R₆ and R₇ are each independently amino group, ester group, urea group, carbamate group, amide group, C₁₋₆ alkyl group, C₁₋₆ alkoxy group, C₃₋₆ cycloalkyl group, C₃₋₆ cycloalkoxy group, C₆₋₁₀ aryl, or C₅₋₁₂ heteroaryl, which is substituted with the 1, 2 or 3 R;
wherein when L₂ is selected from 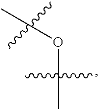 [SMILES: *O*]
B is selected from 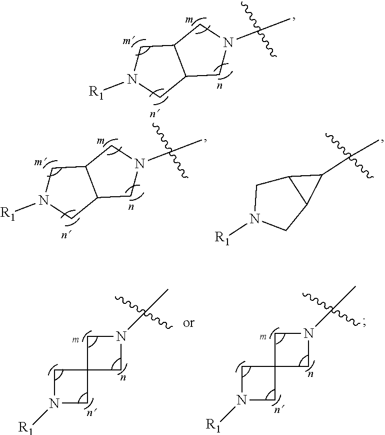
wherein when L₂ is selected from 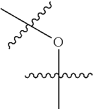 [SMILES: *O*]
and B is selected from 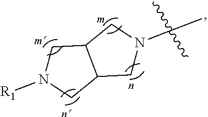
A is selected from 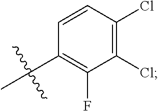
wherein m, n, m′ and n′ are each independently selected from 0, 1, 2, and 3;
C is selected from H, halogen, cyano, amino, ester, urea, ether, carbamate, amide, C₁₋₆ alkyl, C₁₋₆ alkoxy, C₃₋₆ cycloalkyl, C₃₋₆ cycloalkoxy, C₆₋₁₀ aryl, C₅₋₁₂ heteroaryl, and aliphatic heterocycle; or C is amino group, ester group, urea group, ether group, carbamate group, amide group, C₁₋₆ alkyl group, C₁₋₆ alkoxy group, C₃₋₆ cycloalkyl group, C₃₋₆ cycloalkoxy group, C₆₋₁₀ aryl, C₆₋₁₀ heteroaryl, or aliphatic heterocycle, which is substituted by the 1, 2 or 3 R.

2. The compound used as the kinase inhibitor according to claim 1, wherein when L₂ is selected from a single bond, 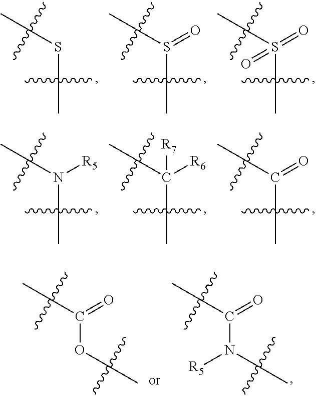
B is selected from:

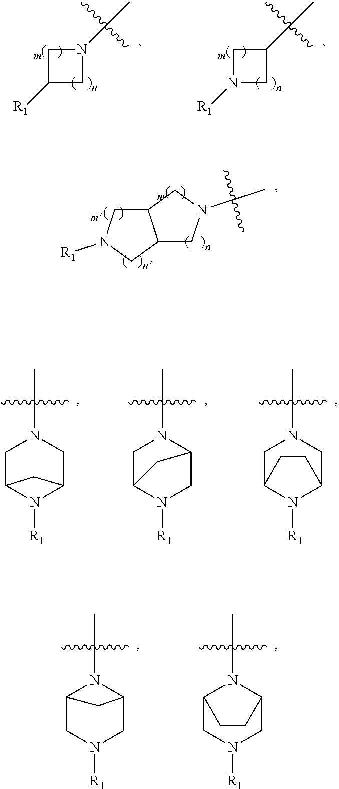

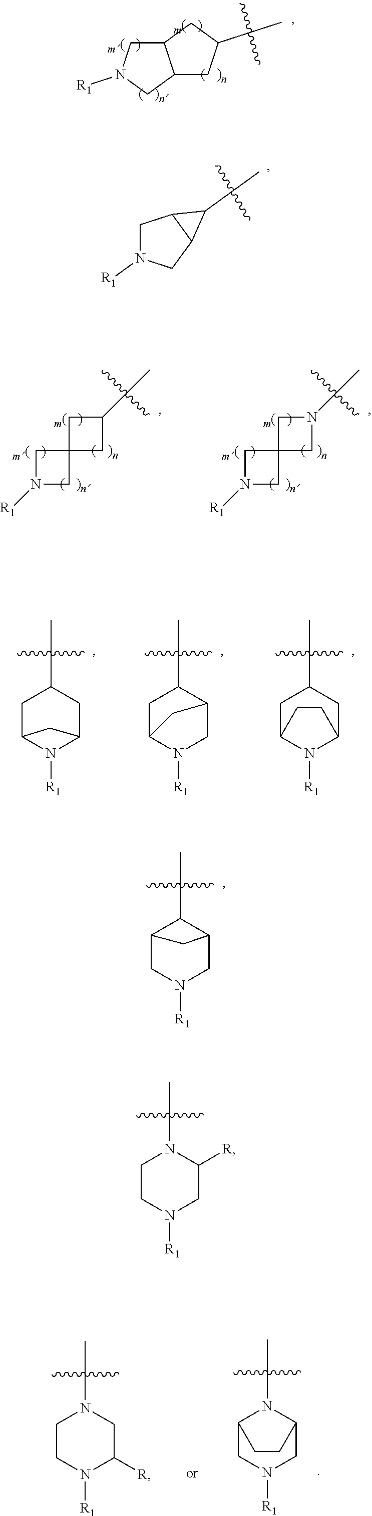

3. The compound used as the kinase inhibitor according to claim 2, wherein R₁ is selected from 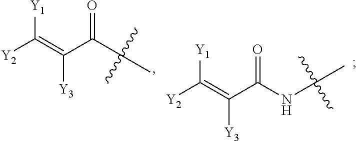
X₂ and X₃ are selected from CH; X₁ is selected from N; L₃ is selected from 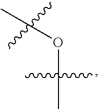 [SMILES: *O*]
C is selected from C₁₋₃ alkyl, 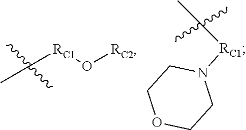
L₁ is selected from 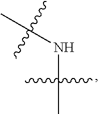 [SMILES: *N*]
A is selected from 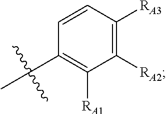
Y₁, Y₂ and Y₃ are selected from hydrogen; R_{A1}, R_{A2} and R_{A3} are each independently selected from hydrogen, halogen and C₁₋₃ alkyl; R_{C1} and R_{C2} are each independently selected from C₁₋₃ alkyl.

4. The compound used as the kinase inhibitor according to claim 1, wherein L₂ is selected from 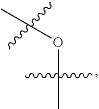 [SMILES: *O*]
B is selected from: 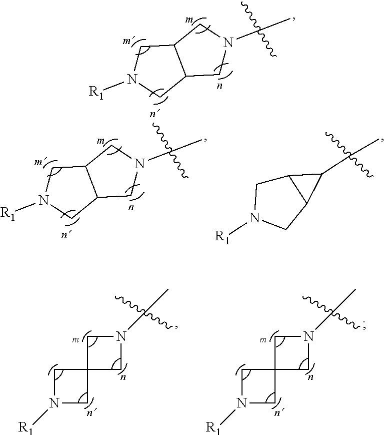
R₁ is selected from 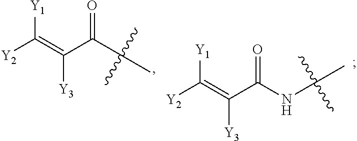
X₂ and X₃ are selected from CH; X₁ is selected from N; L₃ is selected from 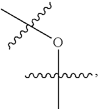 [SMILES: *O*]
C is selected from C₁₋₃ alkyl, 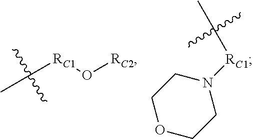
L₁ is selected from 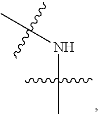 [SMILES: *N*]
A is selected from 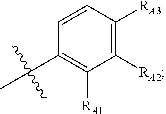
Y₁, Y₂ and Y₃ are selected from hydrogen; R_{A1}, R_{A2} and R_{A3} are each independently selected from hydrogen, halogen and C₁₋₃ alkyl; R_{C1} and R_{C2} are each independently selected from C₁₋₃ alkyl.

5. The compound used as the kinase inhibitor according to claim 1, wherein the compound has a structure shown in formula II: 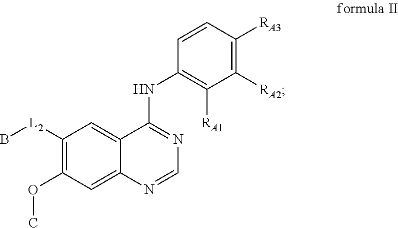
wherein L₂ is selected from 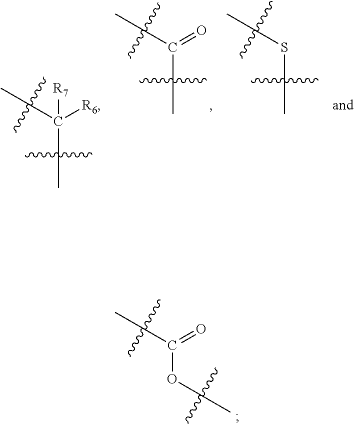
B is selected from 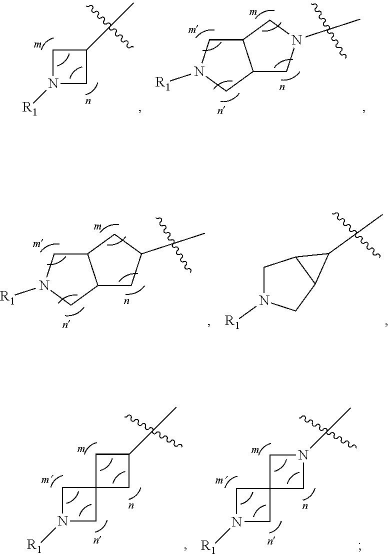
L₂ is selected from a single bond; B is selected from 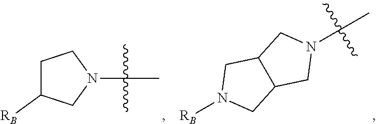
R_{B} is selected from H, 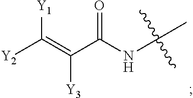
or
L₂ is selected from 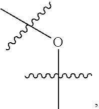 [SMILES: *O*]
B is selected from 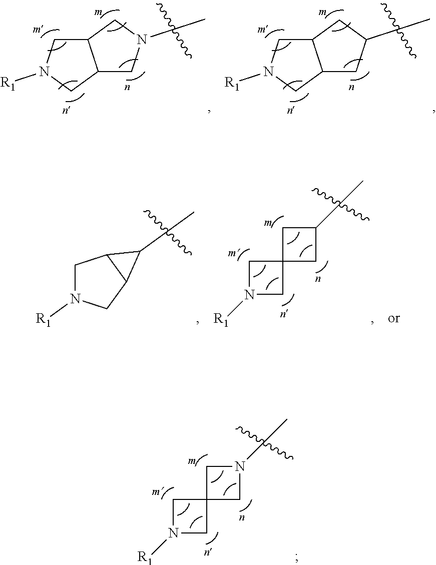
wherein C is selected from C₁₋₃ alkyl, 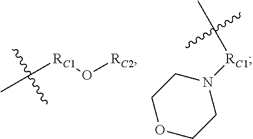
R₁ is selected from 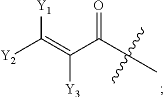
R₆ and R₇ are each independently selected from hydrogen and halogen; Y₁, Y₂, and Y₃ are selected from hydrogen;
R_{A1}, R_{A2}, R_{A3} are each independently selected from hydrogen, halogen, and C₁₋₃ alkyl; R_{C1} and R_{C2} are each independently selected from C₁₋₃ alkyl.

6. The compound used as the kinase inhibitor according to claim 5, wherein L₂ is selected from CF₂, and B is selected from 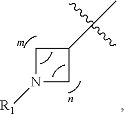
and m and n are both 1 or 2.

7. The compound used as the kinase inhibitor according to claim 5, wherein L₂ is selected from 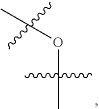 [SMILES: *O*]
and B is selected from 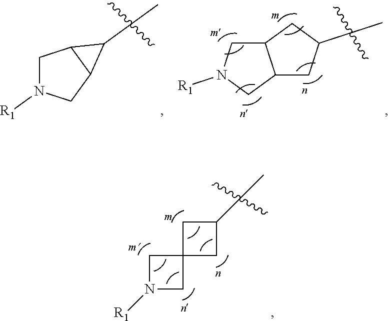
and m, n, m′, and n′ are all 1.

8. The compound used as the kinase inhibitor according to claim 1, wherein the compound is selected from the following compounds:

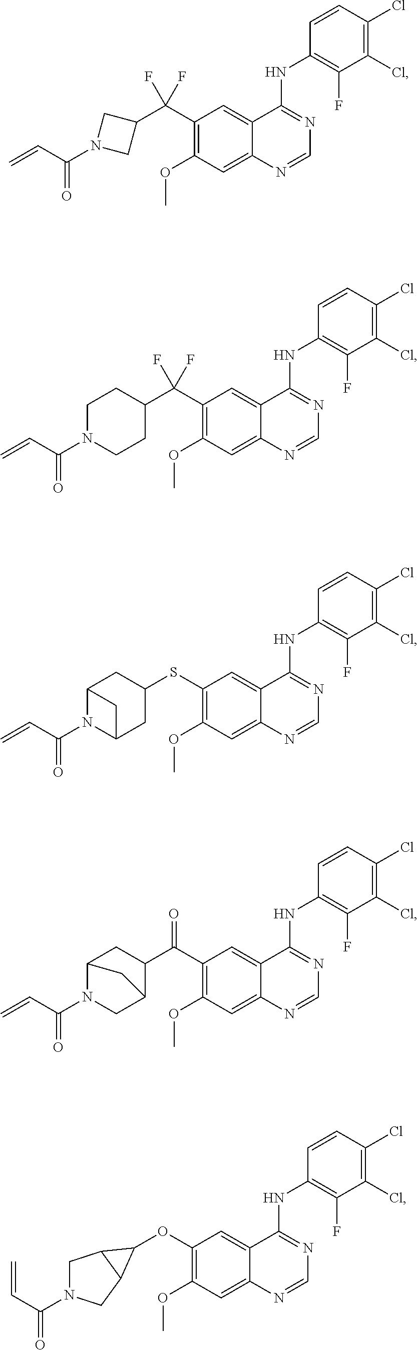 [SMILES: C=CC(=O)N1CC(C(F)(F)c2cc3c(Nc4ccc(Cl)c(Cl)c4F)ncnc3cc2OC)C1 ; C=CC(=O)N1CCC(C(F)(F)c2cc3c(Nc4ccc(Cl)c(Cl)c4F)ncnc3cc2OC)CC1 ; C=CC(=O)N1C2CC(Sc3cc4c(Nc5ccc(Cl)c(Cl)c5F)ncnc4cc3OC)CC1C2 ; C=CC(=O)N1CC2CC1CC2C(=O)c1cc2c(Nc3ccc(Cl)c(Cl)c3F)ncnc2cc1OC ; C=CC(=O)N1CC2C(C1)C2Oc1cc2c(Nc3ccc(Cl)c(Cl)c3F)ncnc2cc1OC]

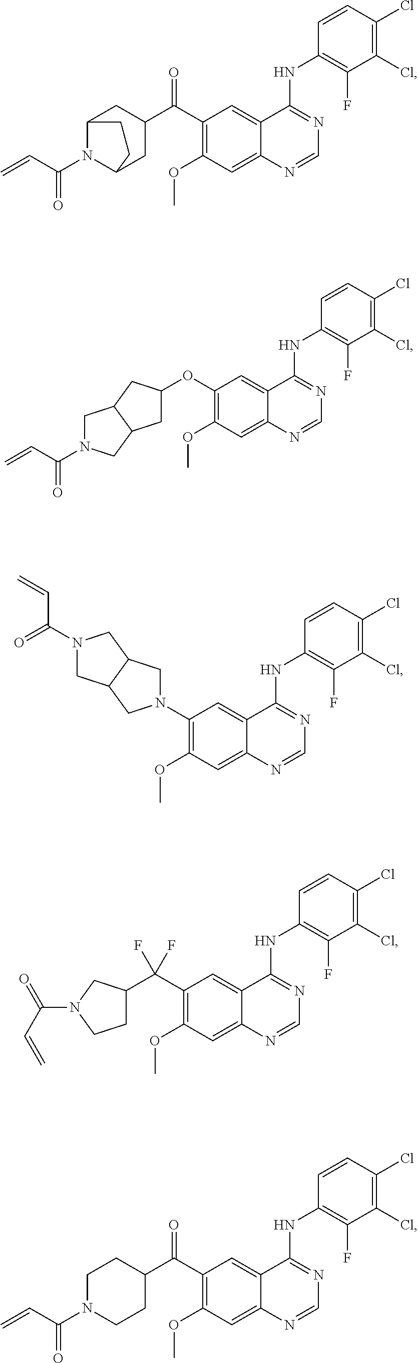 [SMILES: C=CC(=O)N1C2CCC1CC(C(=O)c1cc3c(Nc4ccc(Cl)c(Cl)c4F)ncnc3cc1OC)C2 ; C=CC(=O)N1CC2CC(Oc3cc4c(Nc5ccc(Cl)c(Cl)c5F)ncnc4cc3OC)CC2C1 ; C=CC(=O)N1CC2CN(c3cc4c(Nc5ccc(Cl)c(Cl)c5F)ncnc4cc3OC)CC2C1 ; C=CC(=O)N1CCC(C(F)(F)c2cc3c(Nc4ccc(Cl)c(Cl)c4F)ncnc3cc2OC)C1 ; C=CC(=O)N1CCC(C(=O)c2cc3c(Nc4ccc(Cl)c(Cl)c4F)ncnc3cc2OC)CC1]

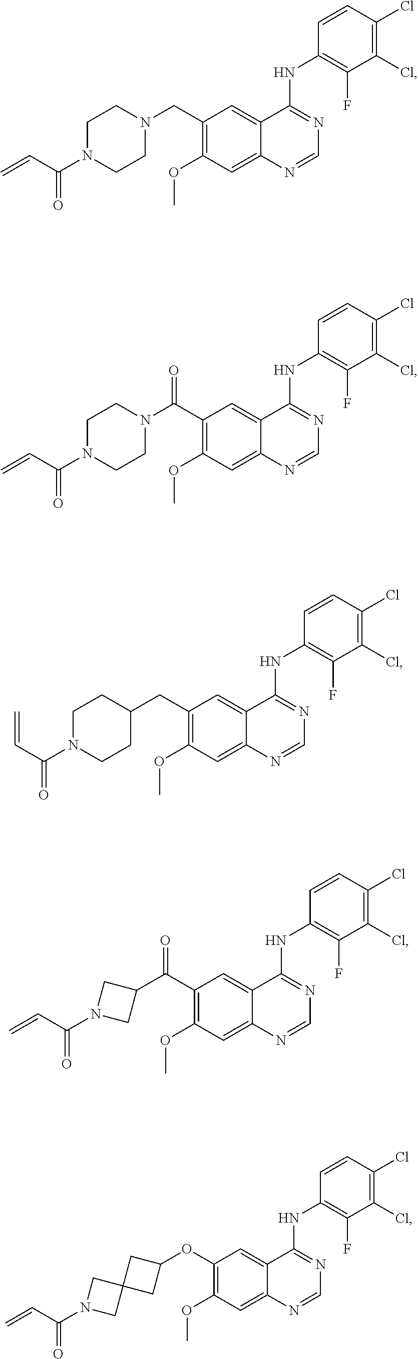 [SMILES: C=CC(=O)N1CCN(Cc2cc3c(Nc4ccc(Cl)c(Cl)c4F)ncnc3cc2OC)CC1 ; C=CC(=O)N1CCN(C(=O)c2cc3c(Nc4ccc(Cl)c(Cl)c4F)ncnc3cc2OC)CC1 ; C=CC(=O)N1CCC(Cc2cc3c(Nc4ccc(Cl)c(Cl)c4F)ncnc3cc2OC)CC1 ; C=CC(=O)N1CC(C(=O)c2cc3c(Nc4ccc(Cl)c(Cl)c4F)ncnc3cc2OC)C1 ; C=CC(=O)N1CC2(CC(Oc3cc4c(Nc5ccc(Cl)c(Cl)c5F)ncnc4cc3OC)C2)C1]

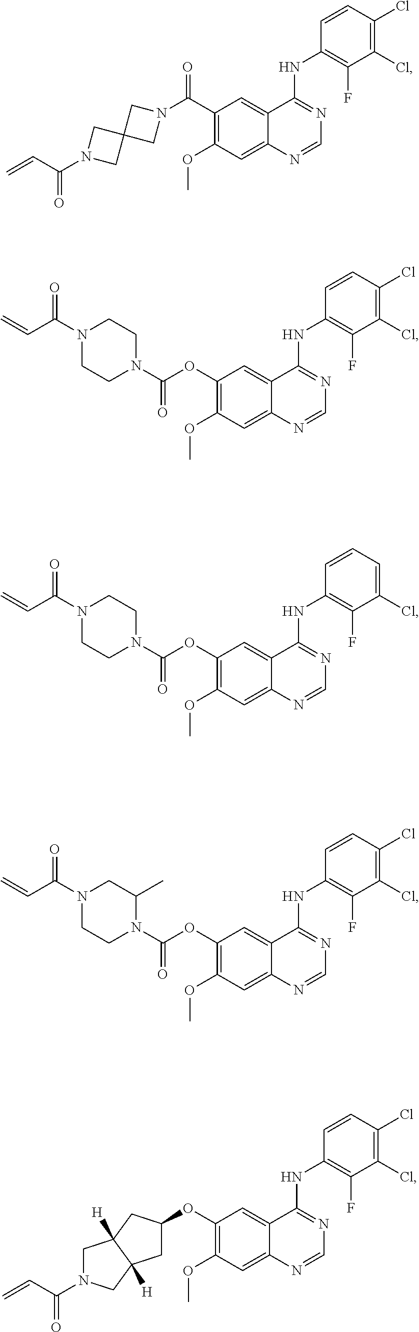 [SMILES: C=CC(=O)N1CC2(C1)CN(C(=O)c1cc3c(Nc4ccc(Cl)c(Cl)c4F)ncnc3cc1OC)C2 ; C=CC(=O)N1CCN(C(=O)Oc2cc3c(Nc4ccc(Cl)c(Cl)c4F)ncnc3cc2OC)CC1 ; C=CC(=O)N1CCN(C(=O)Oc2cc3c(Nc4cccc(Cl)c4F)ncnc3cc2OC)CC1 ; C=CC(=O)N1CCN(C(=O)Oc2cc3c(Nc4ccc(Cl)c(Cl)c4F)ncnc3cc2OC)C(C)C1 ; C=CC(=O)N1C[C@H]2C[C@H](Oc3cc4c(Nc5ccc(Cl)c(Cl)c5F)ncnc4cc3OC)C[C@H]2C1]

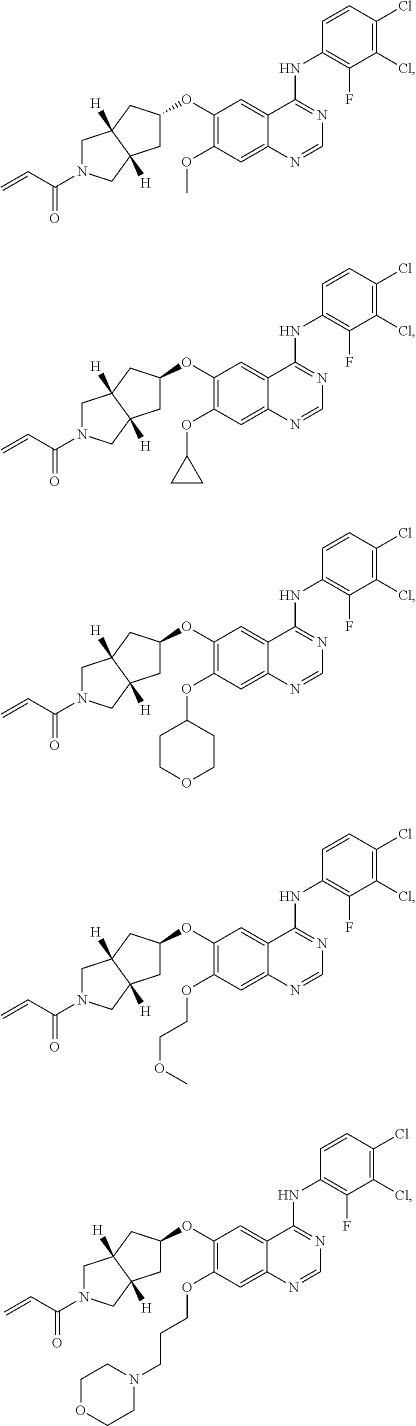 [SMILES: C=CC(=O)N1C[C@H]2C[C@@H](Oc3cc4c(Nc5ccc(Cl)c(Cl)c5F)ncnc4cc3OC)C[C@H]2C1 ; C=CC(=O)N1C[C@H]2C[C@H](Oc3cc4c(Nc5ccc(Cl)c(Cl)c5F)ncnc4cc3OC3CC3)C[C@H]2C1 ; C=CC(=O)N1C[C@H]2C[C@H](Oc3cc4c(Nc5ccc(Cl)c(Cl)c5F)ncnc4cc3OC3CCOCC3)C[C@H]2C1 ; C=CC(=O)N1C[C@H]2C[C@H](Oc3cc4c(Nc5ccc(Cl)c(Cl)c5F)ncnc4cc3OCCOC)C[C@H]2C1 ; C=CC(=O)N1C[C@H]2C[C@H](Oc3cc4c(Nc5ccc(Cl)c(Cl)c5F)ncnc4cc3OCCCN3CCOCC3)C[C@H]2C1]

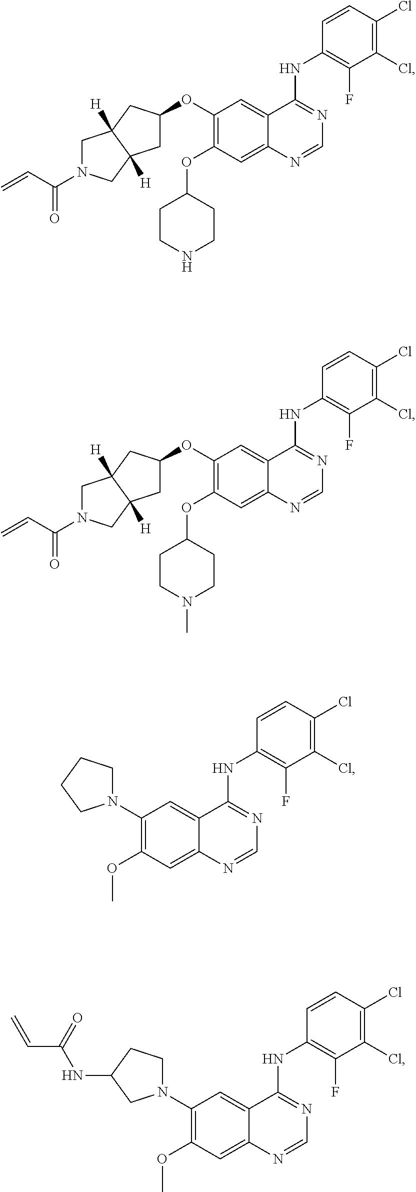 [SMILES: C=CC(=O)N1C[C@H]2C[C@H](Oc3cc4c(Nc5ccc(Cl)c(Cl)c5F)ncnc4cc3OC3CCNCC3)C[C@H]2C1 ; C=CC(=O)N1C[C@H]2C[C@H](Oc3cc4c(Nc5ccc(Cl)c(Cl)c5F)ncnc4cc3OC3CCN(C)CC3)C[C@H]2C1 ; COc1cc2ncnc(Nc3ccc(Cl)c(Cl)c3F)c2cc1N1CCCC1 ; C=CC(=O)NC1CCN(c2cc3c(Nc4ccc(Cl)c(Cl)c4F)ncnc3cc2OC)C1]

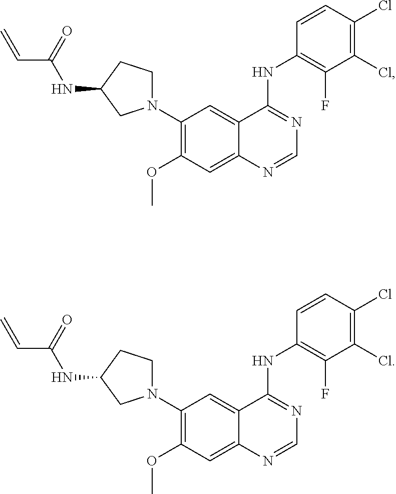 [SMILES: C=CC(=O)N[C@H]1CCN(c2cc3c(Nc4ccc(Cl)c(Cl)c4F)ncnc3cc2OC)C1 ; C=CC(=O)N[C@@H]1CCN(c2cc3c(Nc4ccc(Cl)c(Cl)c4F)ncnc3cc2OC)C1]

9. Use of the compound used as kinase inhibitor according to claim 1 in the preparation of medicine, wherein the medicine is used for treatment of related diseases caused by EGFR mutation and/or Her2 mutation.

10. The use according to claim 9, wherein the EGFR mutation and Her2 mutation are exon 20 insertion mutations.

11. The compound used as the kinase inhibitor according to claim 2, wherein the compound is selected from the following compounds:

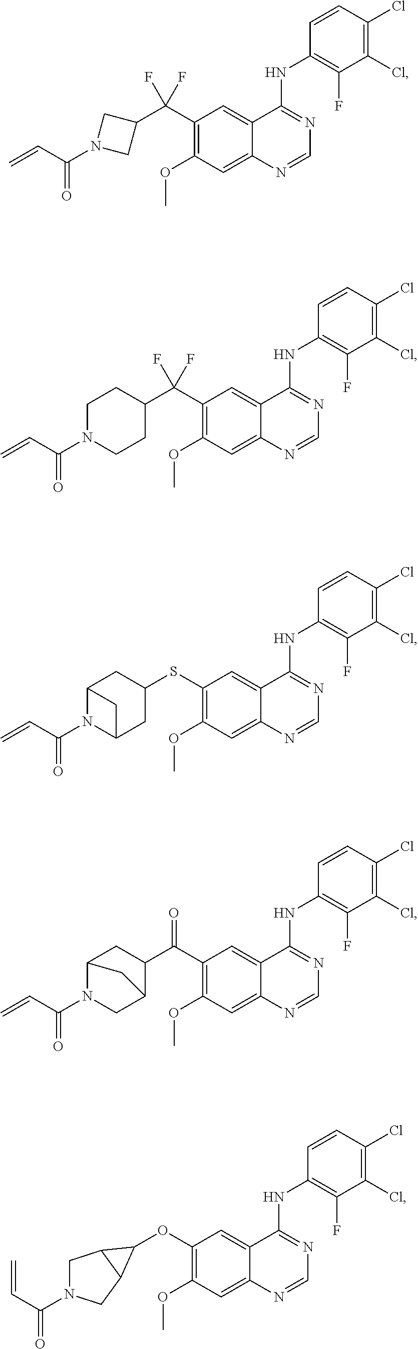 [SMILES: C=CC(=O)N1CC(C(F)(F)c2cc3c(Nc4ccc(Cl)c(Cl)c4F)ncnc3cc2OC)C1 ; C=CC(=O)N1CCC(C(F)(F)c2cc3c(Nc4ccc(Cl)c(Cl)c4F)ncnc3cc2OC)CC1 ; C=CC(=O)N1C2CC(Sc3cc4c(Nc5ccc(Cl)c(Cl)c5F)ncnc4cc3OC)CC1C2 ; C=CC(=O)N1CC2CC1CC2C(=O)c1cc2c(Nc3ccc(Cl)c(Cl)c3F)ncnc2cc1OC ; C=CC(=O)N1CC2C(C1)C2Oc1cc2c(Nc3ccc(Cl)c(Cl)c3F)ncnc2cc1OC]

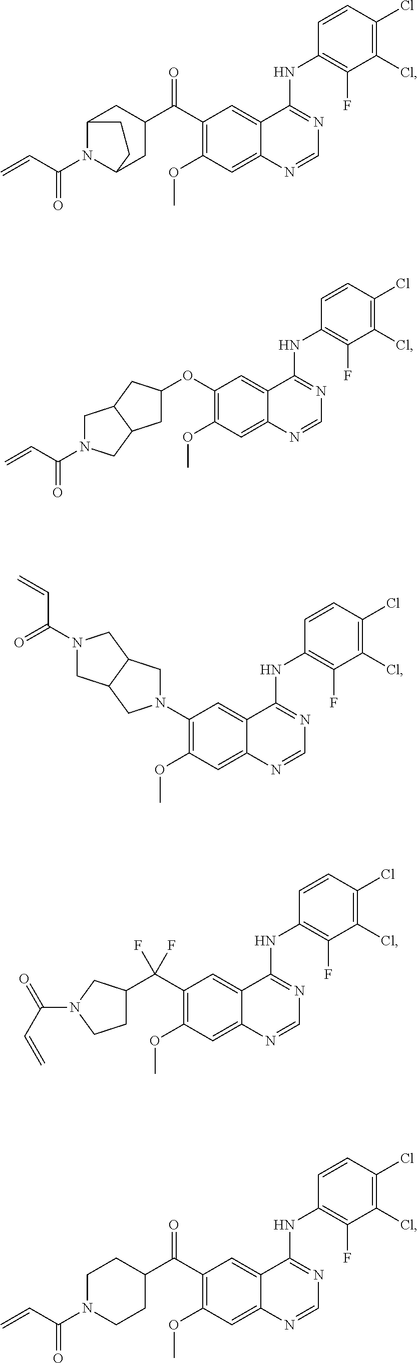 [SMILES: C=CC(=O)N1C2CCC1CC(C(=O)c1cc3c(Nc4ccc(Cl)c(Cl)c4F)ncnc3cc1OC)C2 ; C=CC(=O)N1CC2CC(Oc3cc4c(Nc5ccc(Cl)c(Cl)c5F)ncnc4cc3OC)CC2C1 ; C=CC(=O)N1CC2CN(c3cc4c(Nc5ccc(Cl)c(Cl)c5F)ncnc4cc3OC)CC2C1 ; C=CC(=O)N1CCC(C(F)(F)c2cc3c(Nc4ccc(Cl)c(Cl)c4F)ncnc3cc2OC)C1 ; C=CC(=O)N1CCC(C(=O)c2cc3c(Nc4ccc(Cl)c(Cl)c4F)ncnc3cc2OC)CC1]

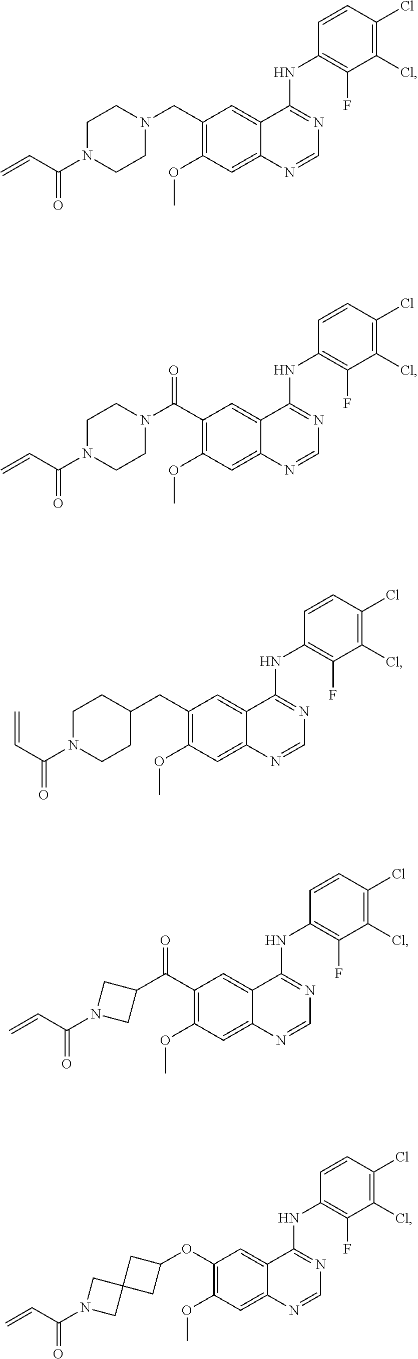 [SMILES: C=CC(=O)N1CCN(Cc2cc3c(Nc4ccc(Cl)c(Cl)c4F)ncnc3cc2OC)CC1 ; C=CC(=O)N1CCN(C(=O)c2cc3c(Nc4ccc(Cl)c(Cl)c4F)ncnc3cc2OC)CC1 ; C=CC(=O)N1CCC(Cc2cc3c(Nc4ccc(Cl)c(Cl)c4F)ncnc3cc2OC)CC1 ; C=CC(=O)N1CC(C(=O)c2cc3c(Nc4ccc(Cl)c(Cl)c4F)ncnc3cc2OC)C1 ; C=CC(=O)N1CC2(CC(Oc3cc4c(Nc5ccc(Cl)c(Cl)c5F)ncnc4cc3OC)C2)C1]

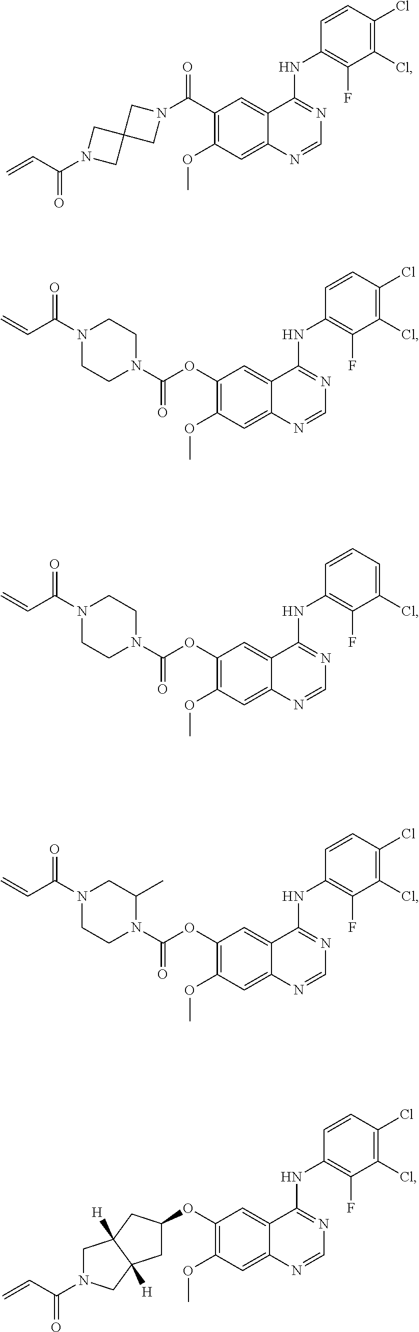 [SMILES: C=CC(=O)N1CC2(C1)CN(C(=O)c1cc3c(Nc4ccc(Cl)c(Cl)c4F)ncnc3cc1OC)C2 ; C=CC(=O)N1CCN(C(=O)Oc2cc3c(Nc4ccc(Cl)c(Cl)c4F)ncnc3cc2OC)CC1 ; C=CC(=O)N1CCN(C(=O)Oc2cc3c(Nc4cccc(Cl)c4F)ncnc3cc2OC)CC1 ; C=CC(=O)N1CCN(C(=O)Oc2cc3c(Nc4ccc(Cl)c(Cl)c4F)ncnc3cc2OC)C(C)C1 ; C=CC(=O)N1C[C@H]2C[C@H](Oc3cc4c(Nc5ccc(Cl)c(Cl)c5F)ncnc4cc3OC)C[C@H]2C1]

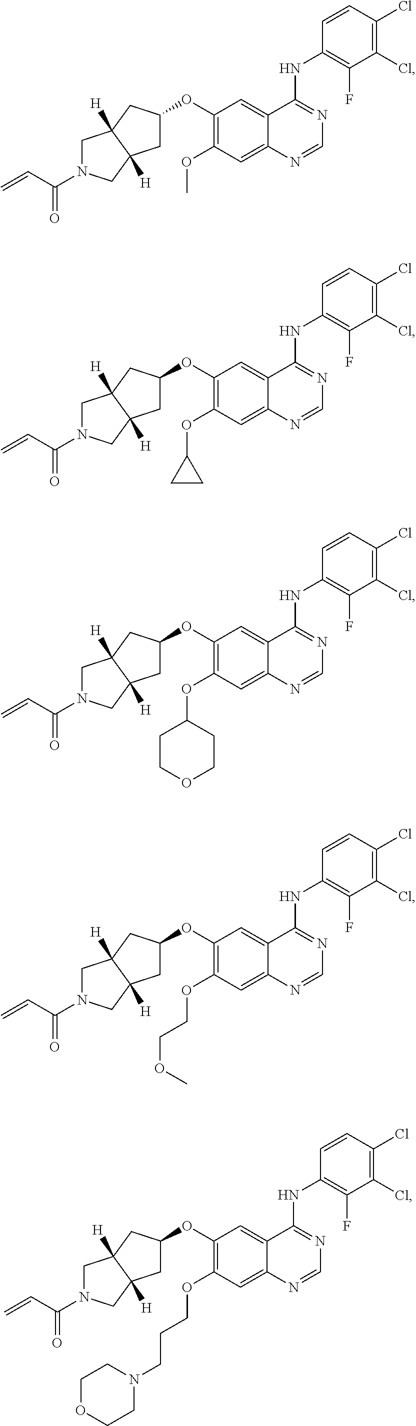 [SMILES: C=CC(=O)N1C[C@H]2C[C@@H](Oc3cc4c(Nc5ccc(Cl)c(Cl)c5F)ncnc4cc3OC)C[C@H]2C1 ; C=CC(=O)N1C[C@H]2C[C@H](Oc3cc4c(Nc5ccc(Cl)c(Cl)c5F)ncnc4cc3OC3CC3)C[C@H]2C1 ; C=CC(=O)N1C[C@H]2C[C@H](Oc3cc4c(Nc5ccc(Cl)c(Cl)c5F)ncnc4cc3OC3CCOCC3)C[C@H]2C1 ; C=CC(=O)N1C[C@H]2C[C@H](Oc3cc4c(Nc5ccc(Cl)c(Cl)c5F)ncnc4cc3OCCOC)C[C@H]2C1 ; C=CC(=O)N1C[C@H]2C[C@H](Oc3cc4c(Nc5ccc(Cl)c(Cl)c5F)ncnc4cc3OCCCN3CCOCC3)C[C@H]2C1]

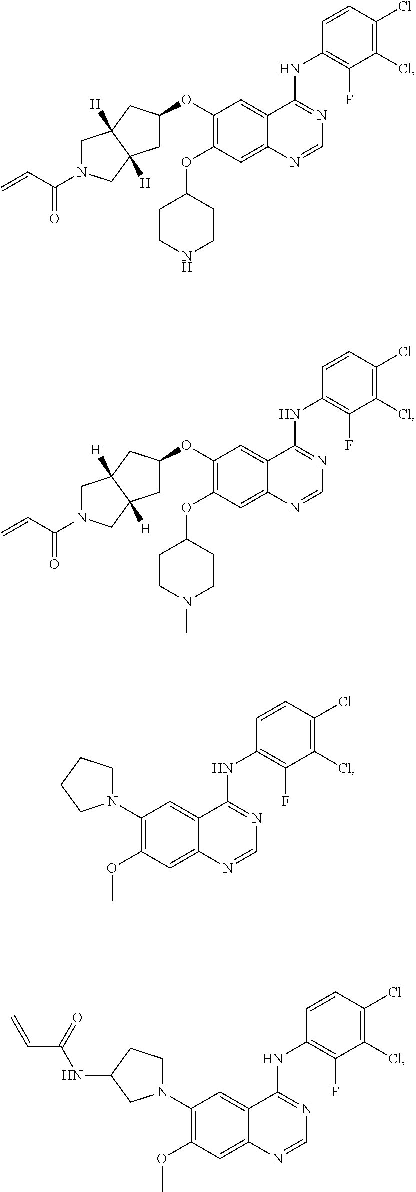 [SMILES: C=CC(=O)N1C[C@H]2C[C@H](Oc3cc4c(Nc5ccc(Cl)c(Cl)c5F)ncnc4cc3OC3CCNCC3)C[C@H]2C1 ; C=CC(=O)N1C[C@H]2C[C@H](Oc3cc4c(Nc5ccc(Cl)c(Cl)c5F)ncnc4cc3OC3CCN(C)CC3)C[C@H]2C1 ; COc1cc2ncnc(Nc3ccc(Cl)c(Cl)c3F)c2cc1N1CCCC1 ; C=CC(=O)NC1CCN(c2cc3c(Nc4ccc(Cl)c(Cl)c4F)ncnc3cc2OC)C1]

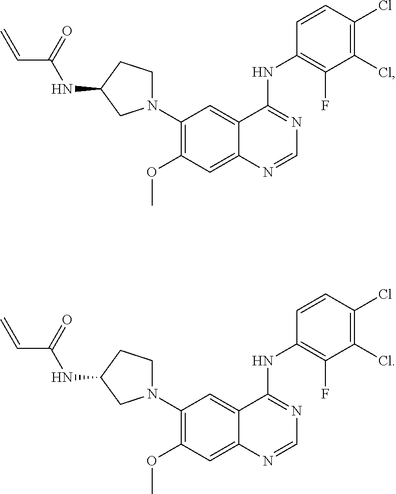 [SMILES: C=CC(=O)N[C@H]1CCN(c2cc3c(Nc4ccc(Cl)c(Cl)c4F)ncnc3cc2OC)C1 ; C=CC(=O)N[C@@H]1CCN(c2cc3c(Nc4ccc(Cl)c(Cl)c4F)ncnc3cc2OC)C1]

12. The compound used as the kinase inhibitor according to claim 3, wherein the compound is selected from the following compounds:

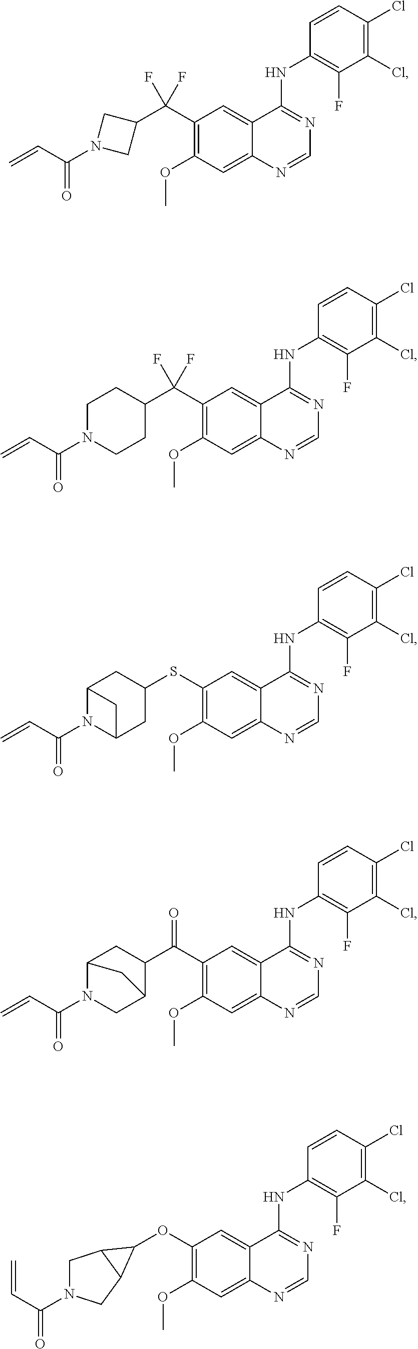 [SMILES: C=CC(=O)N1CC(C(F)(F)c2cc3c(Nc4ccc(Cl)c(Cl)c4F)ncnc3cc2OC)C1 ; C=CC(=O)N1CCC(C(F)(F)c2cc3c(Nc4ccc(Cl)c(Cl)c4F)ncnc3cc2OC)CC1 ; C=CC(=O)N1C2CC(Sc3cc4c(Nc5ccc(Cl)c(Cl)c5F)ncnc4cc3OC)CC1C2 ; C=CC(=O)N1CC2CC1CC2C(=O)c1cc2c(Nc3ccc(Cl)c(Cl)c3F)ncnc2cc1OC ; C=CC(=O)N1CC2C(C1)C2Oc1cc2c(Nc3ccc(Cl)c(Cl)c3F)ncnc2cc1OC]

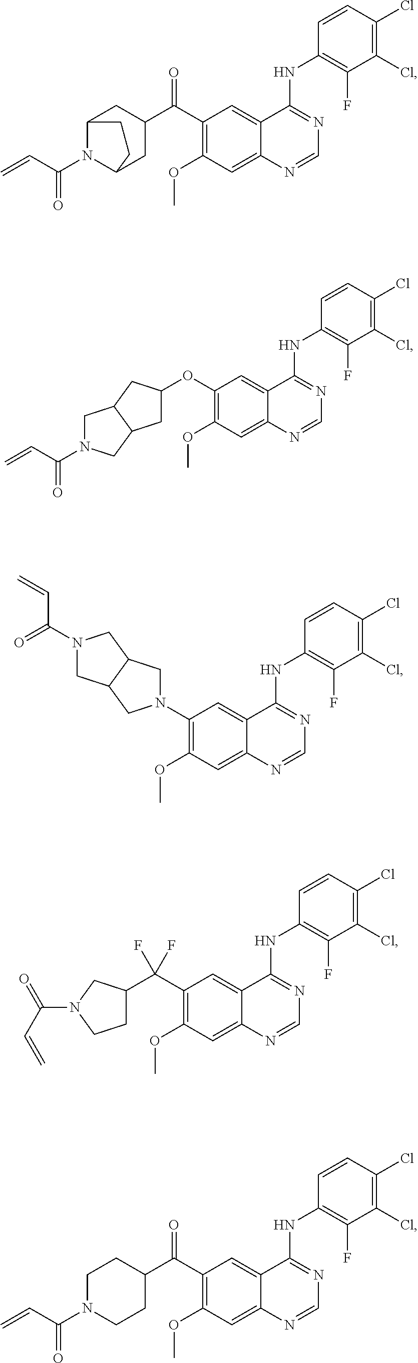 [SMILES: C=CC(=O)N1C2CCC1CC(C(=O)c1cc3c(Nc4ccc(Cl)c(Cl)c4F)ncnc3cc1OC)C2 ; C=CC(=O)N1CC2CC(Oc3cc4c(Nc5ccc(Cl)c(Cl)c5F)ncnc4cc3OC)CC2C1 ; C=CC(=O)N1CC2CN(c3cc4c(Nc5ccc(Cl)c(Cl)c5F)ncnc4cc3OC)CC2C1 ; C=CC(=O)N1CCC(C(F)(F)c2cc3c(Nc4ccc(Cl)c(Cl)c4F)ncnc3cc2OC)C1 ; C=CC(=O)N1CCC(C(=O)c2cc3c(Nc4ccc(Cl)c(Cl)c4F)ncnc3cc2OC)CC1]

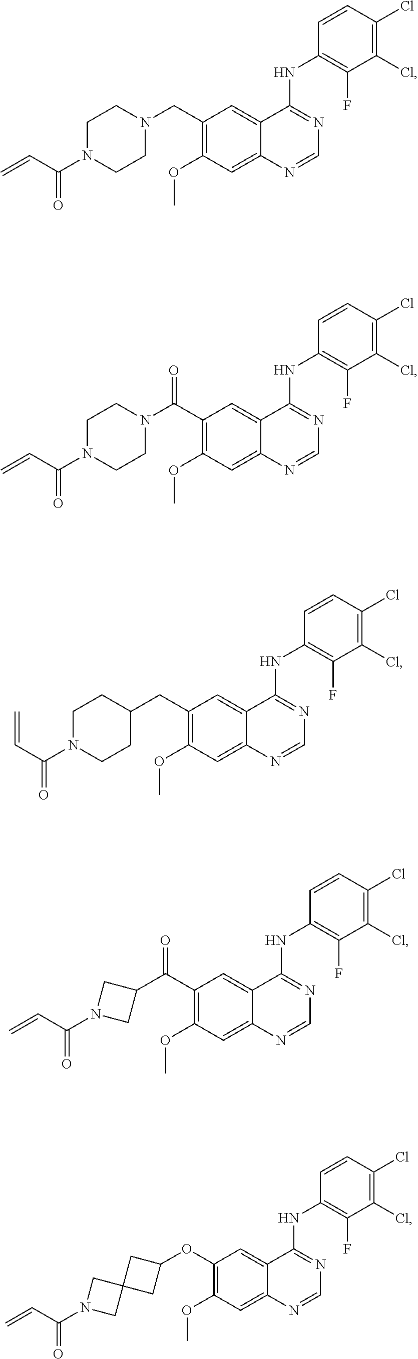 [SMILES: C=CC(=O)N1CCN(Cc2cc3c(Nc4ccc(Cl)c(Cl)c4F)ncnc3cc2OC)CC1 ; C=CC(=O)N1CCN(C(=O)c2cc3c(Nc4ccc(Cl)c(Cl)c4F)ncnc3cc2OC)CC1 ; C=CC(=O)N1CCC(Cc2cc3c(Nc4ccc(Cl)c(Cl)c4F)ncnc3cc2OC)CC1 ; C=CC(=O)N1CC(C(=O)c2cc3c(Nc4ccc(Cl)c(Cl)c4F)ncnc3cc2OC)C1 ; C=CC(=O)N1CC2(CC(Oc3cc4c(Nc5ccc(Cl)c(Cl)c5F)ncnc4cc3OC)C2)C1]

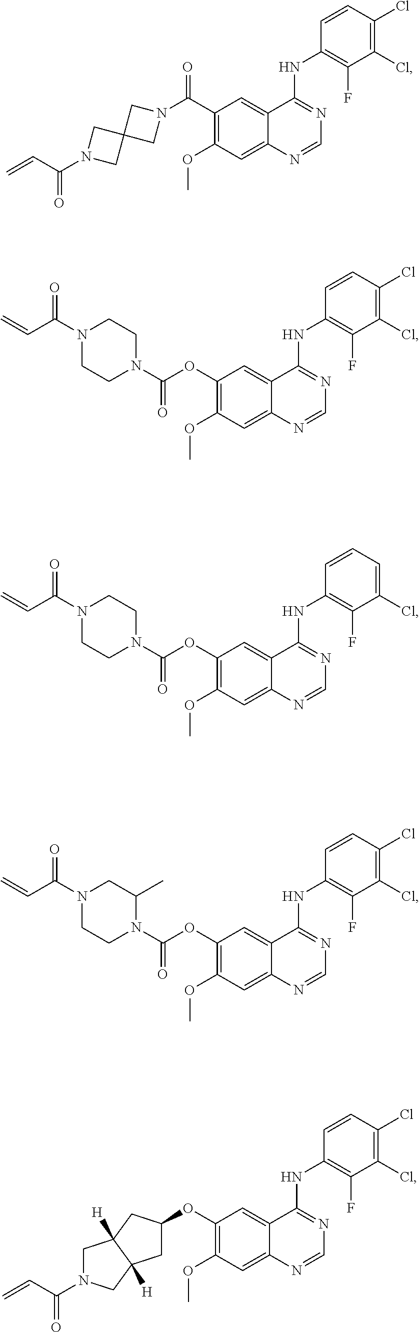 [SMILES: C=CC(=O)N1CC2(C1)CN(C(=O)c1cc3c(Nc4ccc(Cl)c(Cl)c4F)ncnc3cc1OC)C2 ; C=CC(=O)N1CCN(C(=O)Oc2cc3c(Nc4ccc(Cl)c(Cl)c4F)ncnc3cc2OC)CC1 ; C=CC(=O)N1CCN(C(=O)Oc2cc3c(Nc4cccc(Cl)c4F)ncnc3cc2OC)CC1 ; C=CC(=O)N1CCN(C(=O)Oc2cc3c(Nc4ccc(Cl)c(Cl)c4F)ncnc3cc2OC)C(C)C1 ; C=CC(=O)N1C[C@H]2C[C@H](Oc3cc4c(Nc5ccc(Cl)c(Cl)c5F)ncnc4cc3OC)C[C@H]2C1]

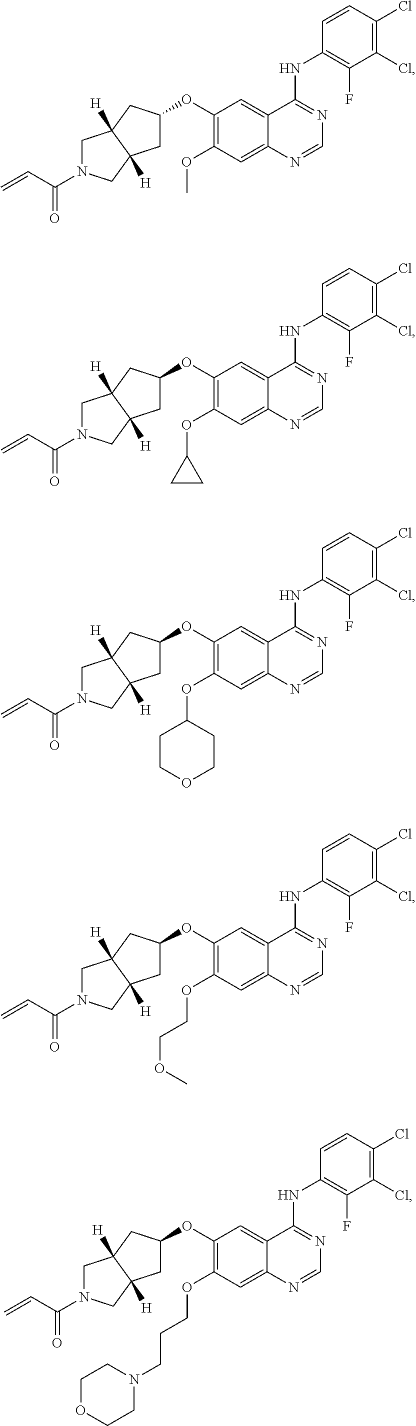 [SMILES: C=CC(=O)N1C[C@H]2C[C@@H](Oc3cc4c(Nc5ccc(Cl)c(Cl)c5F)ncnc4cc3OC)C[C@H]2C1 ; C=CC(=O)N1C[C@H]2C[C@H](Oc3cc4c(Nc5ccc(Cl)c(Cl)c5F)ncnc4cc3OC3CC3)C[C@H]2C1 ; C=CC(=O)N1C[C@H]2C[C@H](Oc3cc4c(Nc5ccc(Cl)c(Cl)c5F)ncnc4cc3OC3CCOCC3)C[C@H]2C1 ; C=CC(=O)N1C[C@H]2C[C@H](Oc3cc4c(Nc5ccc(Cl)c(Cl)c5F)ncnc4cc3OCCOC)C[C@H]2C1 ; C=CC(=O)N1C[C@H]2C[C@H](Oc3cc4c(Nc5ccc(Cl)c(Cl)c5F)ncnc4cc3OCCCN3CCOCC3)C[C@H]2C1]

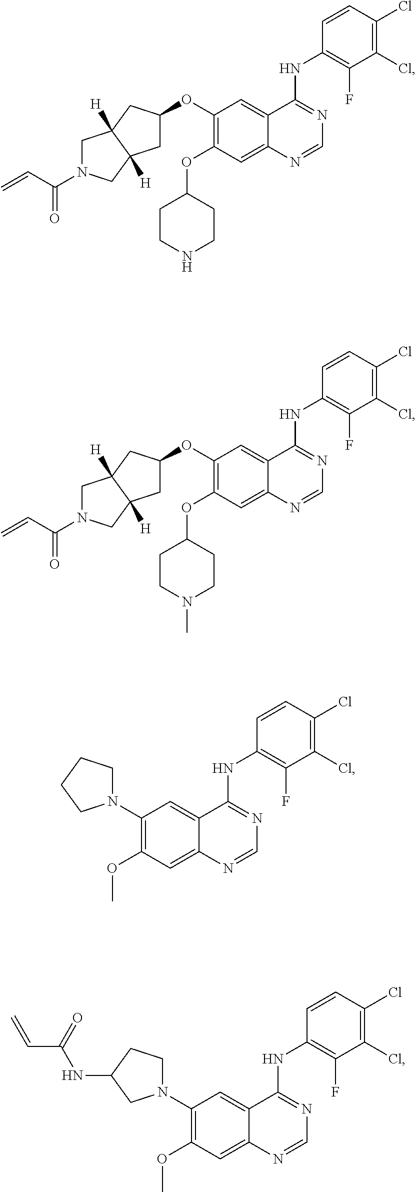 [SMILES: C=CC(=O)N1C[C@H]2C[C@H](Oc3cc4c(Nc5ccc(Cl)c(Cl)c5F)ncnc4cc3OC3CCNCC3)C[C@H]2C1 ; C=CC(=O)N1C[C@H]2C[C@H](Oc3cc4c(Nc5ccc(Cl)c(Cl)c5F)ncnc4cc3OC3CCN(C)CC3)C[C@H]2C1 ; COc1cc2ncnc(Nc3ccc(Cl)c(Cl)c3F)c2cc1N1CCCC1 ; C=CC(=O)NC1CCN(c2cc3c(Nc4ccc(Cl)c(Cl)c4F)ncnc3cc2OC)C1]

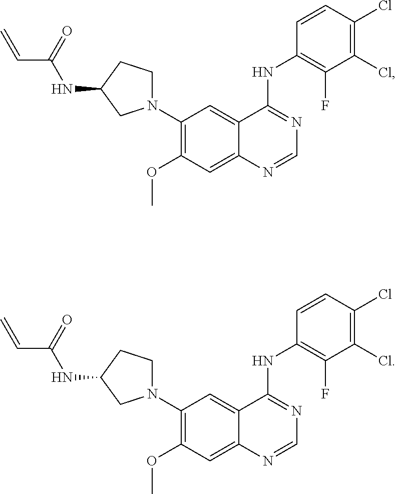 [SMILES: C=CC(=O)N[C@H]1CCN(c2cc3c(Nc4ccc(Cl)c(Cl)c4F)ncnc3cc2OC)C1 ; C=CC(=O)N[C@@H]1CCN(c2cc3c(Nc4ccc(Cl)c(Cl)c4F)ncnc3cc2OC)C1]

13. The compound used as the kinase inhibitor according to claim 4, wherein the compound is selected from the following compounds:

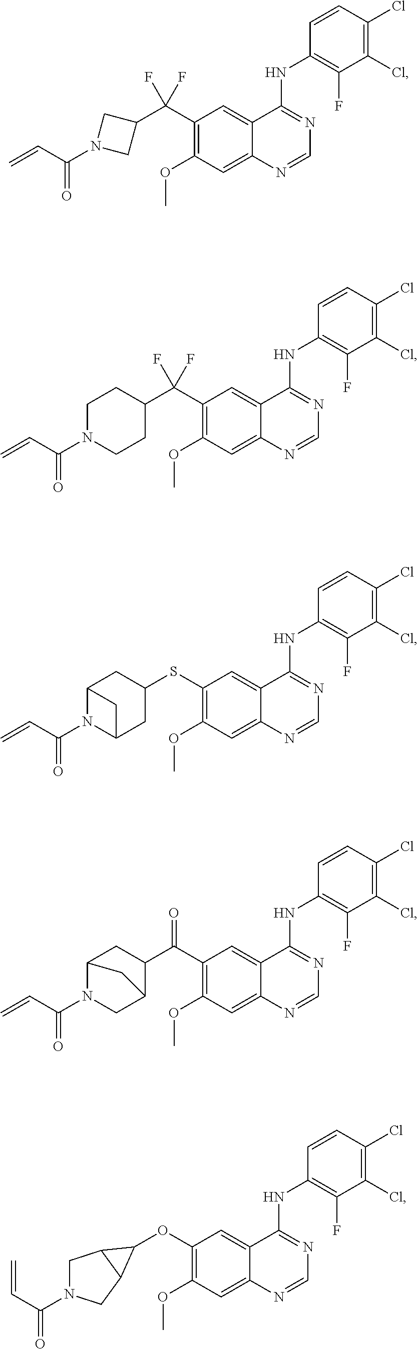 [SMILES: C=CC(=O)N1CC(C(F)(F)c2cc3c(Nc4ccc(Cl)c(Cl)c4F)ncnc3cc2OC)C1 ; C=CC(=O)N1CCC(C(F)(F)c2cc3c(Nc4ccc(Cl)c(Cl)c4F)ncnc3cc2OC)CC1 ; C=CC(=O)N1C2CC(Sc3cc4c(Nc5ccc(Cl)c(Cl)c5F)ncnc4cc3OC)CC1C2 ; C=CC(=O)N1CC2CC1CC2C(=O)c1cc2c(Nc3ccc(Cl)c(Cl)c3F)ncnc2cc1OC ; C=CC(=O)N1CC2C(C1)C2Oc1cc2c(Nc3ccc(Cl)c(Cl)c3F)ncnc2cc1OC]

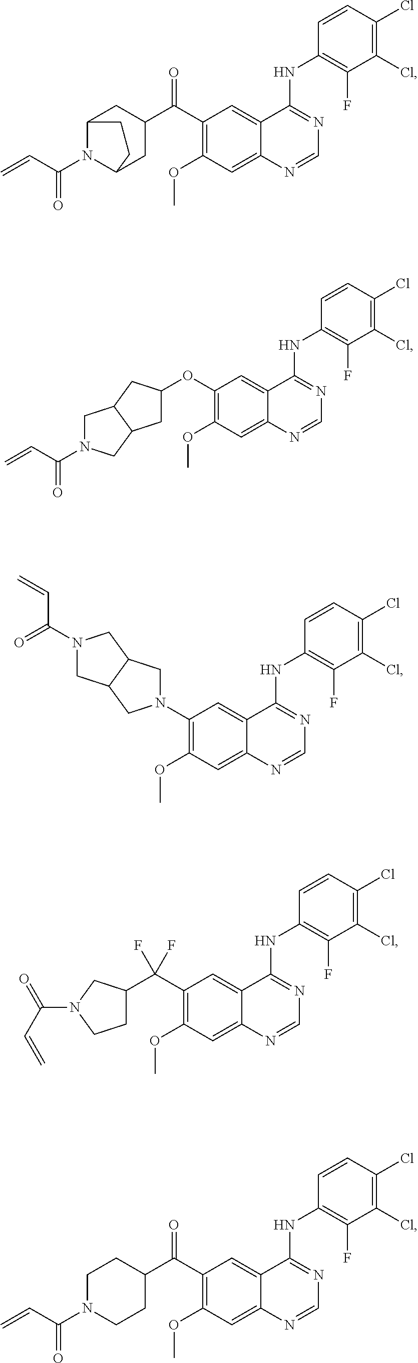 [SMILES: C=CC(=O)N1C2CCC1CC(C(=O)c1cc3c(Nc4ccc(Cl)c(Cl)c4F)ncnc3cc1OC)C2 ; C=CC(=O)N1CC2CC(Oc3cc4c(Nc5ccc(Cl)c(Cl)c5F)ncnc4cc3OC)CC2C1 ; C=CC(=O)N1CC2CN(c3cc4c(Nc5ccc(Cl)c(Cl)c5F)ncnc4cc3OC)CC2C1 ; C=CC(=O)N1CCC(C(F)(F)c2cc3c(Nc4ccc(Cl)c(Cl)c4F)ncnc3cc2OC)C1 ; C=CC(=O)N1CCC(C(=O)c2cc3c(Nc4ccc(Cl)c(Cl)c4F)ncnc3cc2OC)CC1]

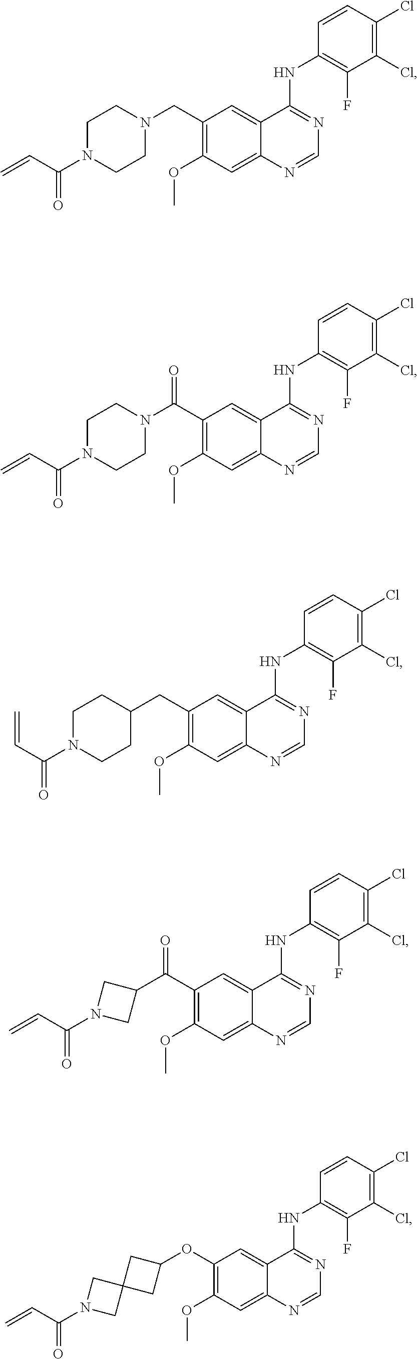 [SMILES: C=CC(=O)N1CCN(Cc2cc3c(Nc4ccc(Cl)c(Cl)c4F)ncnc3cc2OC)CC1 ; C=CC(=O)N1CCN(C(=O)c2cc3c(Nc4ccc(Cl)c(Cl)c4F)ncnc3cc2OC)CC1 ; C=CC(=O)N1CCC(Cc2cc3c(Nc4ccc(Cl)c(Cl)c4F)ncnc3cc2OC)CC1 ; C=CC(=O)N1CC(C(=O)c2cc3c(Nc4ccc(Cl)c(Cl)c4F)ncnc3cc2OC)C1 ; C=CC(=O)N1CC2(CC(Oc3cc4c(Nc5ccc(Cl)c(Cl)c5F)ncnc4cc3OC)C2)C1]

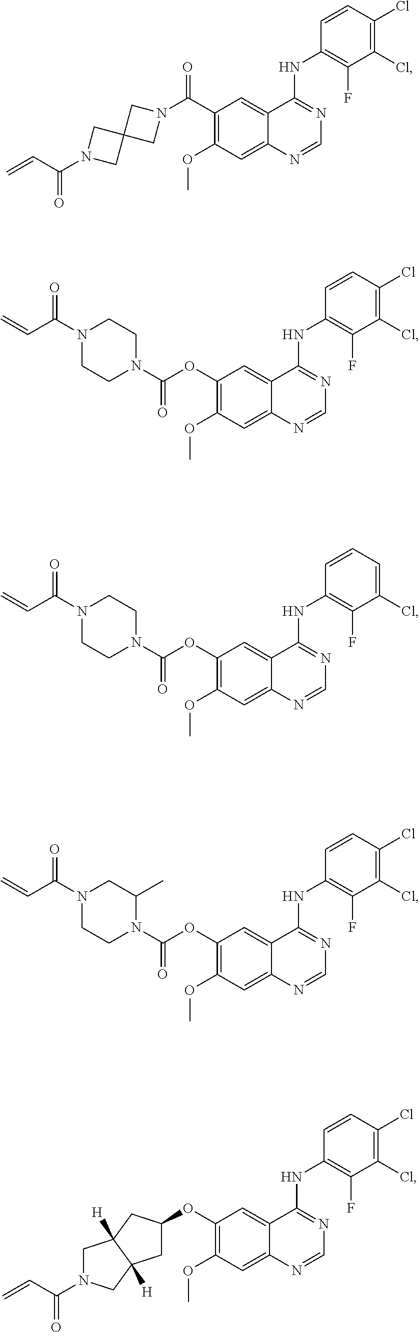 [SMILES: C=CC(=O)N1CC2(C1)CN(C(=O)c1cc3c(Nc4ccc(Cl)c(Cl)c4F)ncnc3cc1OC)C2 ; C=CC(=O)N1CCN(C(=O)Oc2cc3c(Nc4ccc(Cl)c(Cl)c4F)ncnc3cc2OC)CC1 ; C=CC(=O)N1CCN(C(=O)Oc2cc3c(Nc4cccc(Cl)c4F)ncnc3cc2OC)CC1 ; C=CC(=O)N1CCN(C(=O)Oc2cc3c(Nc4ccc(Cl)c(Cl)c4F)ncnc3cc2OC)C(C)C1 ; C=CC(=O)N1C[C@H]2C[C@H](Oc3cc4c(Nc5ccc(Cl)c(Cl)c5F)ncnc4cc3OC)C[C@H]2C1]

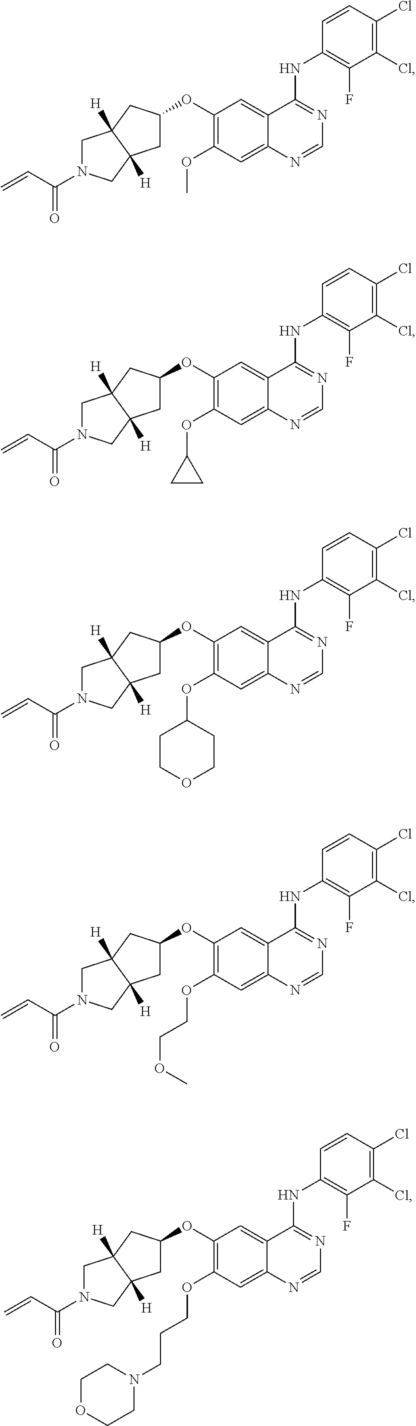 [SMILES: C=CC(=O)N1C[C@H]2C[C@@H](Oc3cc4c(Nc5ccc(Cl)c(Cl)c5F)ncnc4cc3OC)C[C@H]2C1 ; C=CC(=O)N1C[C@H]2C[C@H](Oc3cc4c(Nc5ccc(Cl)c(Cl)c5F)ncnc4cc3OC3CC3)C[C@H]2C1 ; C=CC(=O)N1C[C@H]2C[C@H](Oc3cc4c(Nc5ccc(Cl)c(Cl)c5F)ncnc4cc3OC3CCOCC3)C[C@H]2C1 ; C=CC(=O)N1C[C@H]2C[C@H](Oc3cc4c(Nc5ccc(Cl)c(Cl)c5F)ncnc4cc3OCCOC)C[C@H]2C1 ; C=CC(=O)N1C[C@H]2C[C@H](Oc3cc4c(Nc5ccc(Cl)c(Cl)c5F)ncnc4cc3OCCCN3CCOCC3)C[C@H]2C1]

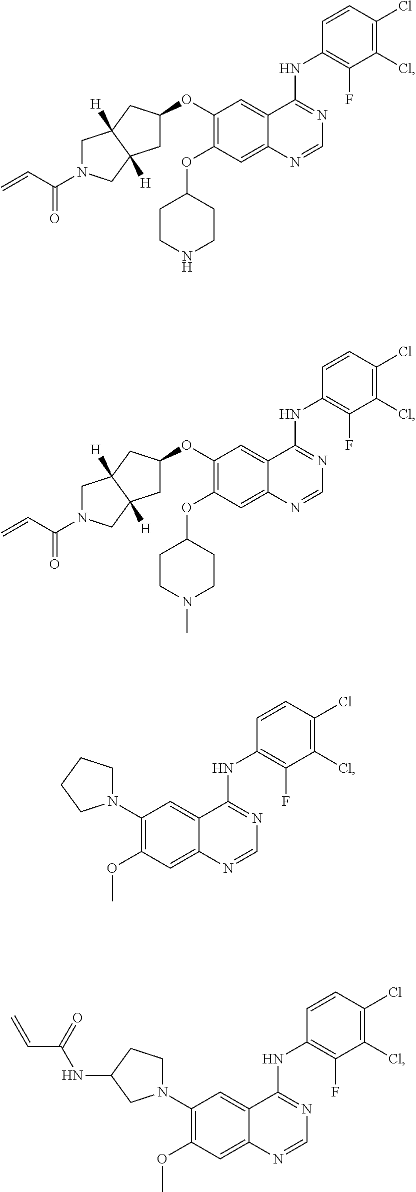 [SMILES: C=CC(=O)N1C[C@H]2C[C@H](Oc3cc4c(Nc5ccc(Cl)c(Cl)c5F)ncnc4cc3OC3CCNCC3)C[C@H]2C1 ; C=CC(=O)N1C[C@H]2C[C@H](Oc3cc4c(Nc5ccc(Cl)c(Cl)c5F)ncnc4cc3OC3CCN(C)CC3)C[C@H]2C1 ; COc1cc2ncnc(Nc3ccc(Cl)c(Cl)c3F)c2cc1N1CCCC1 ; C=CC(=O)NC1CCN(c2cc3c(Nc4ccc(Cl)c(Cl)c4F)ncnc3cc2OC)C1]

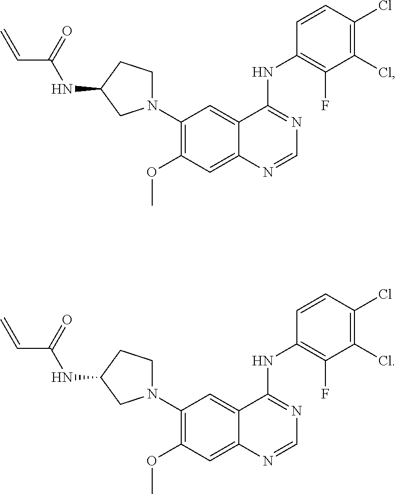 [SMILES: C=CC(=O)N[C@H]1CCN(c2cc3c(Nc4ccc(Cl)c(Cl)c4F)ncnc3cc2OC)C1 ; C=CC(=O)N[C@@H]1CCN(c2cc3c(Nc4ccc(Cl)c(Cl)c4F)ncnc3cc2OC)C1]

14. The compound used as the kinase inhibitor according to claim 5, wherein the compound is selected from the following compounds:

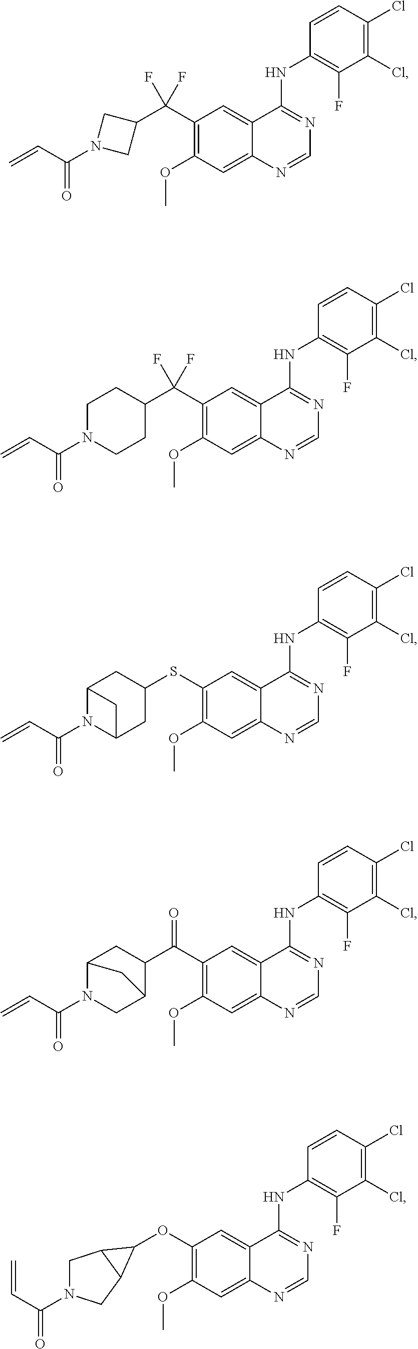 [SMILES: C=CC(=O)N1CC(C(F)(F)c2cc3c(Nc4ccc(Cl)c(Cl)c4F)ncnc3cc2OC)C1 ; C=CC(=O)N1CCC(C(F)(F)c2cc3c(Nc4ccc(Cl)c(Cl)c4F)ncnc3cc2OC)CC1 ; C=CC(=O)N1C2CC(Sc3cc4c(Nc5ccc(Cl)c(Cl)c5F)ncnc4cc3OC)CC1C2 ; C=CC(=O)N1CC2CC1CC2C(=O)c1cc2c(Nc3ccc(Cl)c(Cl)c3F)ncnc2cc1OC ; C=CC(=O)N1CC2C(C1)C2Oc1cc2c(Nc3ccc(Cl)c(Cl)c3F)ncnc2cc1OC]

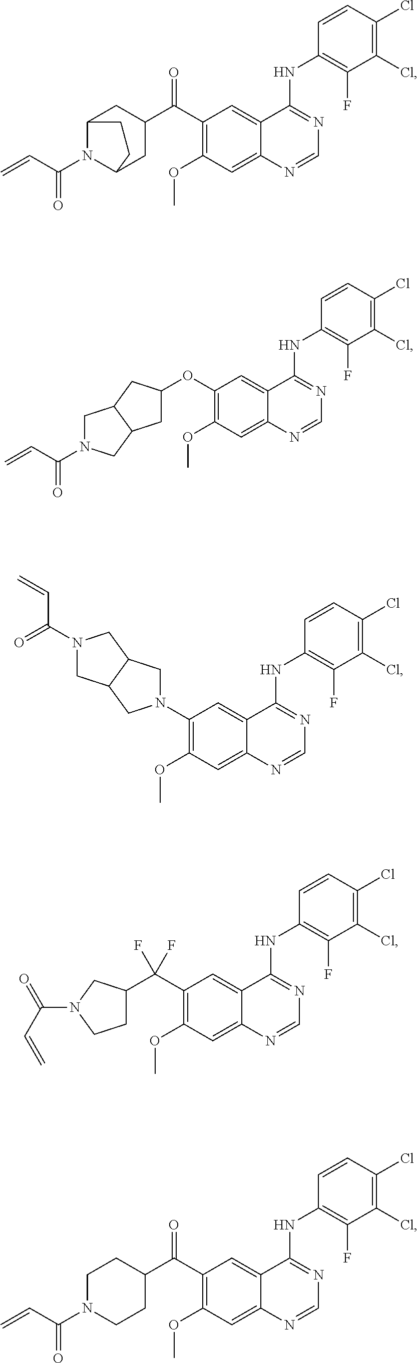 [SMILES: C=CC(=O)N1C2CCC1CC(C(=O)c1cc3c(Nc4ccc(Cl)c(Cl)c4F)ncnc3cc1OC)C2 ; C=CC(=O)N1CC2CC(Oc3cc4c(Nc5ccc(Cl)c(Cl)c5F)ncnc4cc3OC)CC2C1 ; C=CC(=O)N1CC2CN(c3cc4c(Nc5ccc(Cl)c(Cl)c5F)ncnc4cc3OC)CC2C1 ; C=CC(=O)N1CCC(C(F)(F)c2cc3c(Nc4ccc(Cl)c(Cl)c4F)ncnc3cc2OC)C1 ; C=CC(=O)N1CCC(C(=O)c2cc3c(Nc4ccc(Cl)c(Cl)c4F)ncnc3cc2OC)CC1]

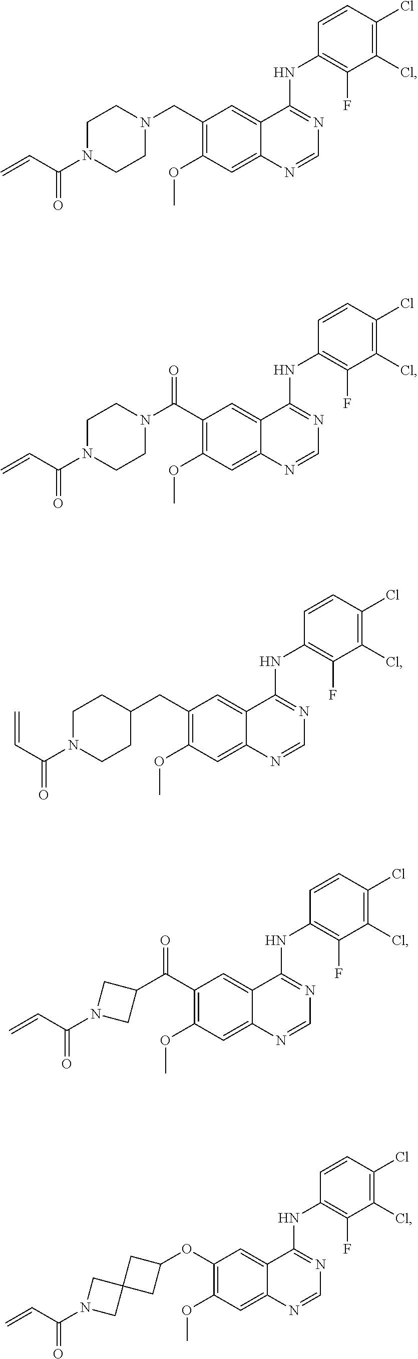 [SMILES: C=CC(=O)N1CCN(Cc2cc3c(Nc4ccc(Cl)c(Cl)c4F)ncnc3cc2OC)CC1 ; C=CC(=O)N1CCN(C(=O)c2cc3c(Nc4ccc(Cl)c(Cl)c4F)ncnc3cc2OC)CC1 ; C=CC(=O)N1CCC(Cc2cc3c(Nc4ccc(Cl)c(Cl)c4F)ncnc3cc2OC)CC1 ; C=CC(=O)N1CC(C(=O)c2cc3c(Nc4ccc(Cl)c(Cl)c4F)ncnc3cc2OC)C1 ; C=CC(=O)N1CC2(CC(Oc3cc4c(Nc5ccc(Cl)c(Cl)c5F)ncnc4cc3OC)C2)C1]

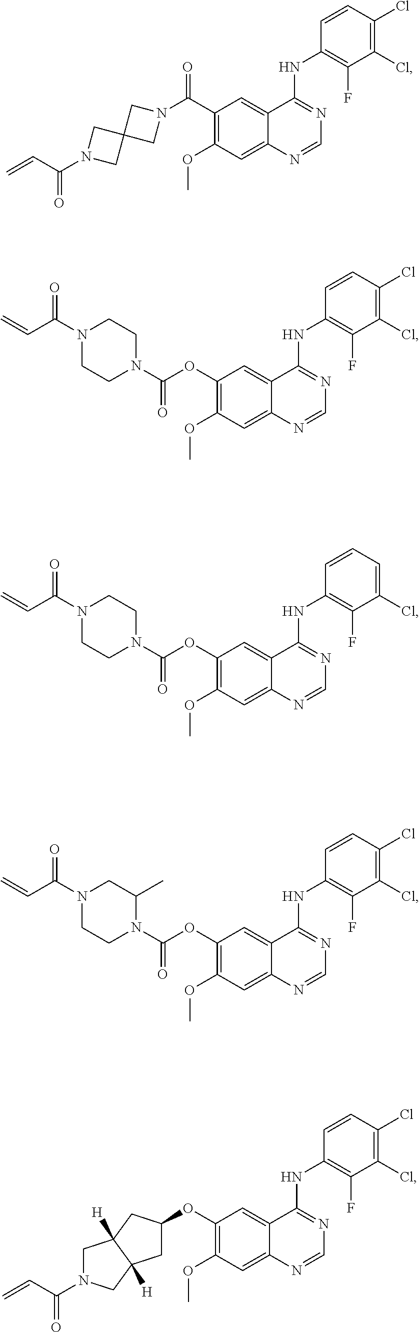 [SMILES: C=CC(=O)N1CC2(C1)CN(C(=O)c1cc3c(Nc4ccc(Cl)c(Cl)c4F)ncnc3cc1OC)C2 ; C=CC(=O)N1CCN(C(=O)Oc2cc3c(Nc4ccc(Cl)c(Cl)c4F)ncnc3cc2OC)CC1 ; C=CC(=O)N1CCN(C(=O)Oc2cc3c(Nc4cccc(Cl)c4F)ncnc3cc2OC)CC1 ; C=CC(=O)N1CCN(C(=O)Oc2cc3c(Nc4ccc(Cl)c(Cl)c4F)ncnc3cc2OC)C(C)C1 ; C=CC(=O)N1C[C@H]2C[C@H](Oc3cc4c(Nc5ccc(Cl)c(Cl)c5F)ncnc4cc3OC)C[C@H]2C1]

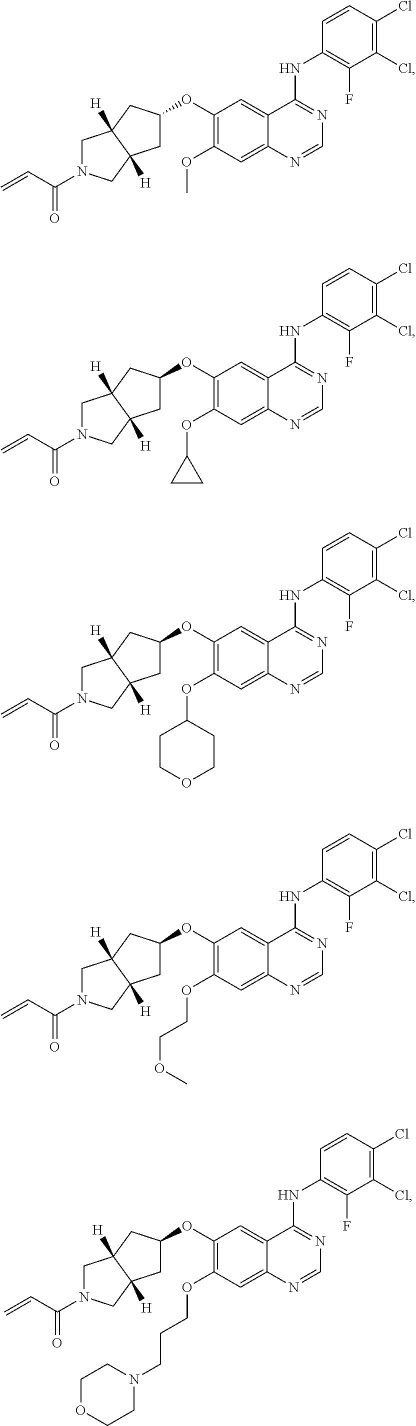 [SMILES: C=CC(=O)N1C[C@H]2C[C@@H](Oc3cc4c(Nc5ccc(Cl)c(Cl)c5F)ncnc4cc3OC)C[C@H]2C1 ; C=CC(=O)N1C[C@H]2C[C@H](Oc3cc4c(Nc5ccc(Cl)c(Cl)c5F)ncnc4cc3OC3CC3)C[C@H]2C1 ; C=CC(=O)N1C[C@H]2C[C@H](Oc3cc4c(Nc5ccc(Cl)c(Cl)c5F)ncnc4cc3OC3CCOCC3)C[C@H]2C1 ; C=CC(=O)N1C[C@H]2C[C@H](Oc3cc4c(Nc5ccc(Cl)c(Cl)c5F)ncnc4cc3OCCOC)C[C@H]2C1 ; C=CC(=O)N1C[C@H]2C[C@H](Oc3cc4c(Nc5ccc(Cl)c(Cl)c5F)ncnc4cc3OCCCN3CCOCC3)C[C@H]2C1]

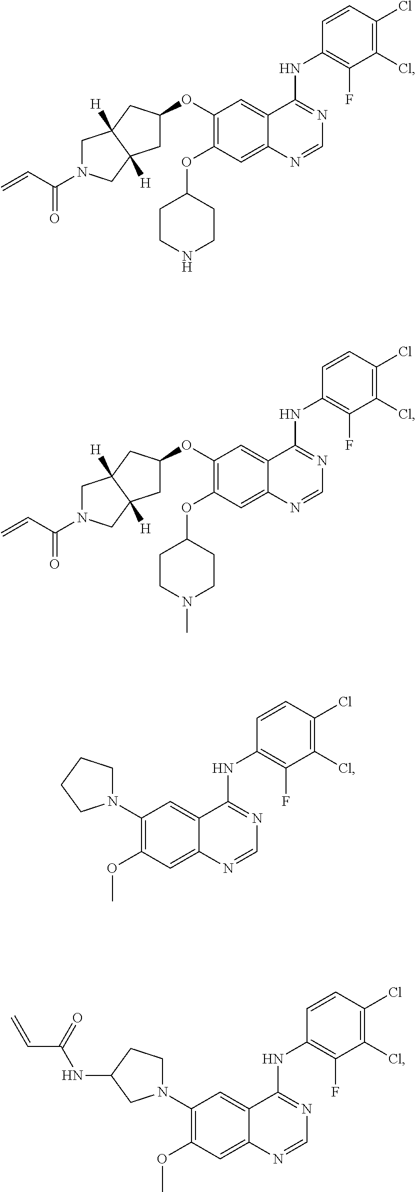 [SMILES: C=CC(=O)N1C[C@H]2C[C@H](Oc3cc4c(Nc5ccc(Cl)c(Cl)c5F)ncnc4cc3OC3CCNCC3)C[C@H]2C1 ; C=CC(=O)N1C[C@H]2C[C@H](Oc3cc4c(Nc5ccc(Cl)c(Cl)c5F)ncnc4cc3OC3CCN(C)CC3)C[C@H]2C1 ; COc1cc2ncnc(Nc3ccc(Cl)c(Cl)c3F)c2cc1N1CCCC1 ; C=CC(=O)NC1CCN(c2cc3c(Nc4ccc(Cl)c(Cl)c4F)ncnc3cc2OC)C1]

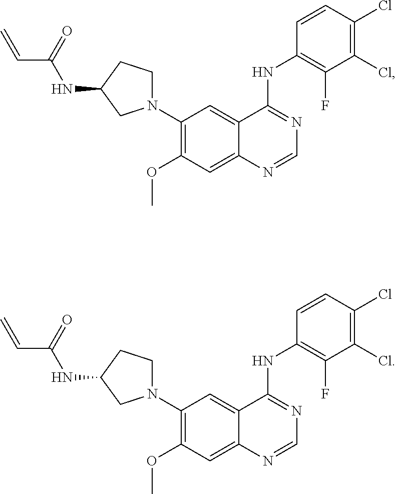 [SMILES: C=CC(=O)N[C@H]1CCN(c2cc3c(Nc4ccc(Cl)c(Cl)c4F)ncnc3cc2OC)C1 ; C=CC(=O)N[C@@H]1CCN(c2cc3c(Nc4ccc(Cl)c(Cl)c4F)ncnc3cc2OC)C1]

15. The compound used as the kinase inhibitor according to claim 6, wherein the compound is selected from the following compounds:

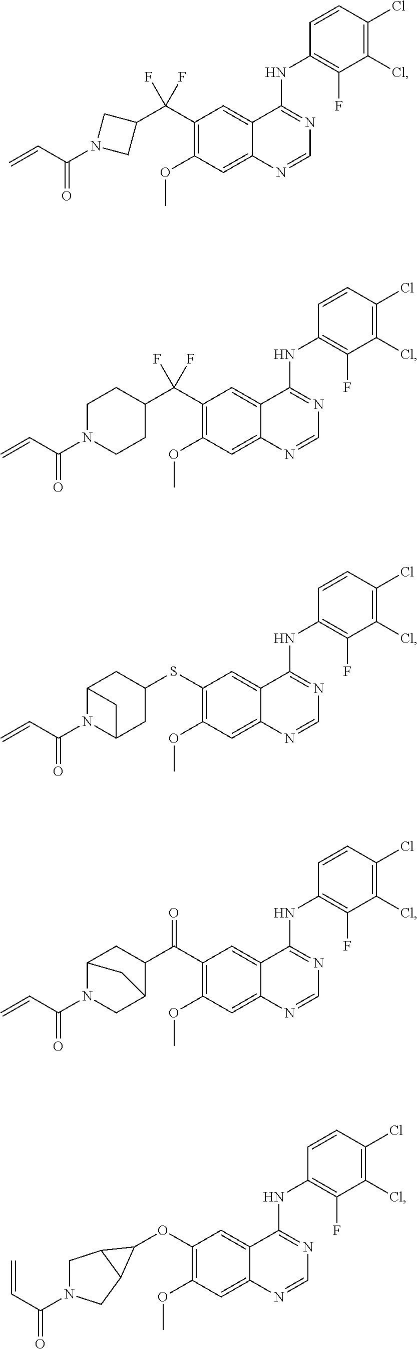 [SMILES: C=CC(=O)N1CC(C(F)(F)c2cc3c(Nc4ccc(Cl)c(Cl)c4F)ncnc3cc2OC)C1 ; C=CC(=O)N1CCC(C(F)(F)c2cc3c(Nc4ccc(Cl)c(Cl)c4F)ncnc3cc2OC)CC1 ; C=CC(=O)N1C2CC(Sc3cc4c(Nc5ccc(Cl)c(Cl)c5F)ncnc4cc3OC)CC1C2 ; C=CC(=O)N1CC2CC1CC2C(=O)c1cc2c(Nc3ccc(Cl)c(Cl)c3F)ncnc2cc1OC ; C=CC(=O)N1CC2C(C1)C2Oc1cc2c(Nc3ccc(Cl)c(Cl)c3F)ncnc2cc1OC]

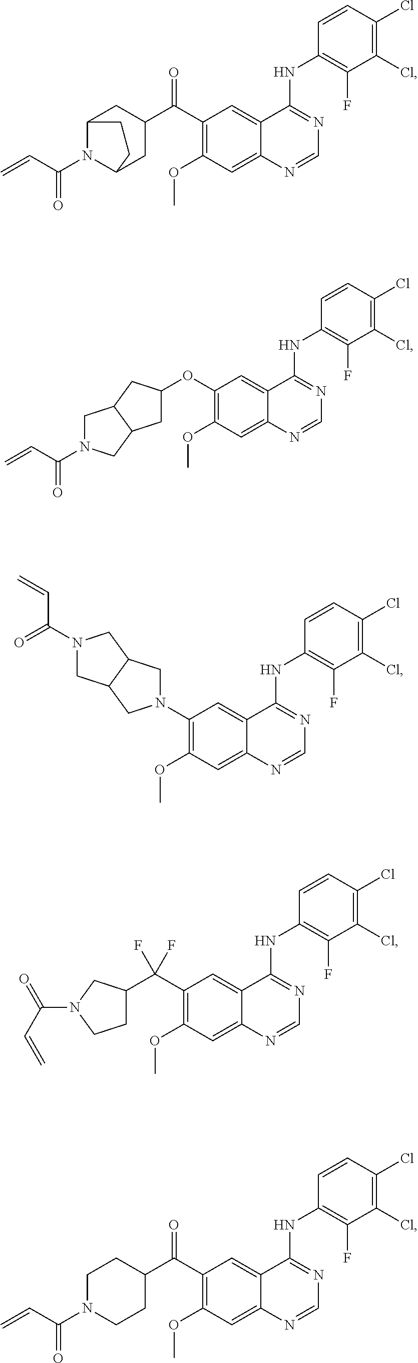 [SMILES: C=CC(=O)N1C2CCC1CC(C(=O)c1cc3c(Nc4ccc(Cl)c(Cl)c4F)ncnc3cc1OC)C2 ; C=CC(=O)N1CC2CC(Oc3cc4c(Nc5ccc(Cl)c(Cl)c5F)ncnc4cc3OC)CC2C1 ; C=CC(=O)N1CC2CN(c3cc4c(Nc5ccc(Cl)c(Cl)c5F)ncnc4cc3OC)CC2C1 ; C=CC(=O)N1CCC(C(F)(F)c2cc3c(Nc4ccc(Cl)c(Cl)c4F)ncnc3cc2OC)C1 ; C=CC(=O)N1CCC(C(=O)c2cc3c(Nc4ccc(Cl)c(Cl)c4F)ncnc3cc2OC)CC1]

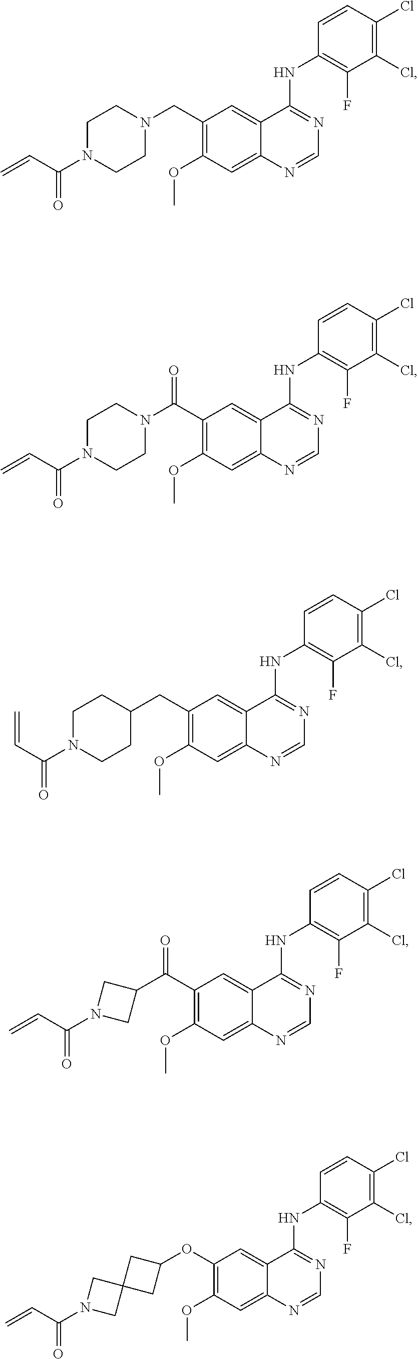 [SMILES: C=CC(=O)N1CCN(Cc2cc3c(Nc4ccc(Cl)c(Cl)c4F)ncnc3cc2OC)CC1 ; C=CC(=O)N1CCN(C(=O)c2cc3c(Nc4ccc(Cl)c(Cl)c4F)ncnc3cc2OC)CC1 ; C=CC(=O)N1CCC(Cc2cc3c(Nc4ccc(Cl)c(Cl)c4F)ncnc3cc2OC)CC1 ; C=CC(=O)N1CC(C(=O)c2cc3c(Nc4ccc(Cl)c(Cl)c4F)ncnc3cc2OC)C1 ; C=CC(=O)N1CC2(CC(Oc3cc4c(Nc5ccc(Cl)c(Cl)c5F)ncnc4cc3OC)C2)C1]

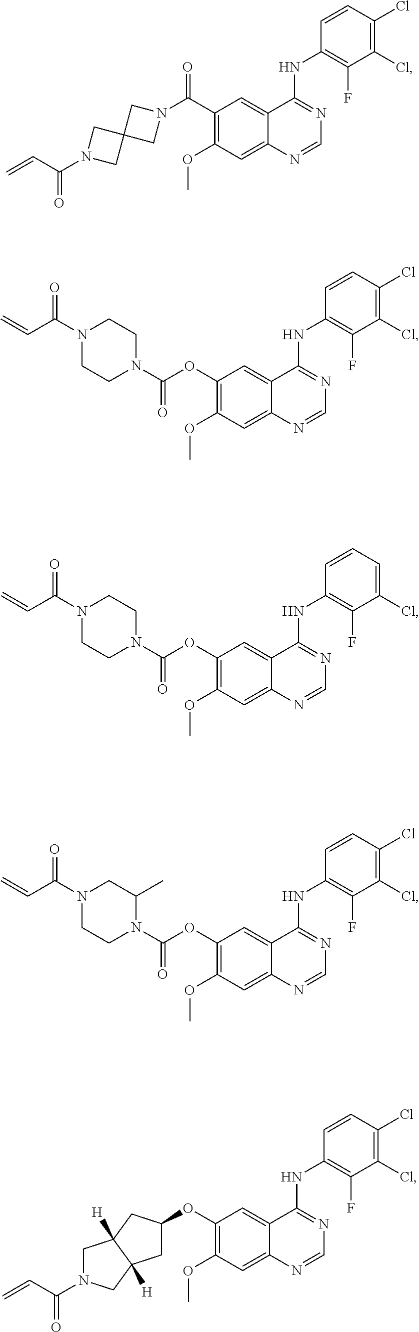 [SMILES: C=CC(=O)N1CC2(C1)CN(C(=O)c1cc3c(Nc4ccc(Cl)c(Cl)c4F)ncnc3cc1OC)C2 ; C=CC(=O)N1CCN(C(=O)Oc2cc3c(Nc4ccc(Cl)c(Cl)c4F)ncnc3cc2OC)CC1 ; C=CC(=O)N1CCN(C(=O)Oc2cc3c(Nc4cccc(Cl)c4F)ncnc3cc2OC)CC1 ; C=CC(=O)N1CCN(C(=O)Oc2cc3c(Nc4ccc(Cl)c(Cl)c4F)ncnc3cc2OC)C(C)C1 ; C=CC(=O)N1C[C@H]2C[C@H](Oc3cc4c(Nc5ccc(Cl)c(Cl)c5F)ncnc4cc3OC)C[C@H]2C1]

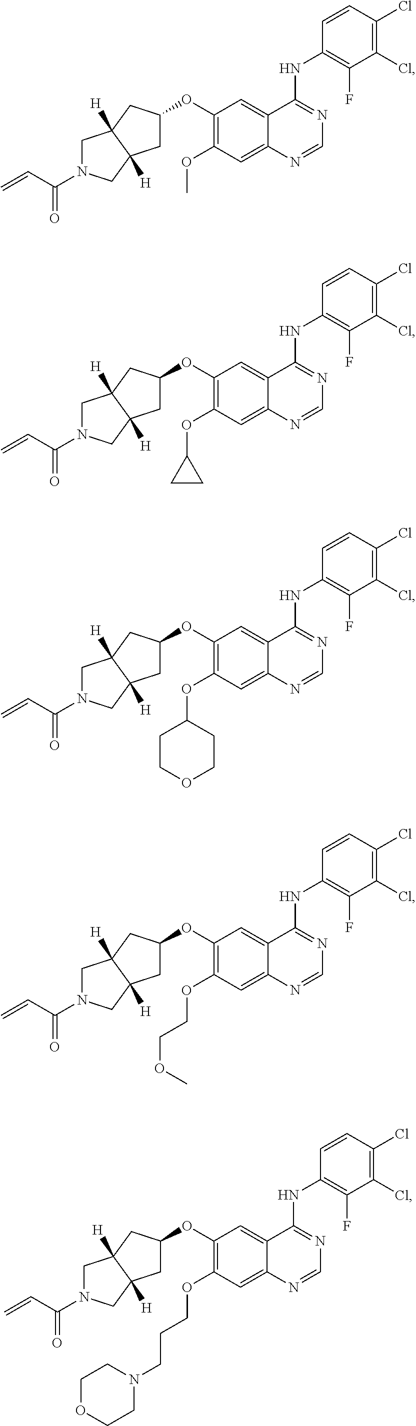 [SMILES: C=CC(=O)N1C[C@H]2C[C@@H](Oc3cc4c(Nc5ccc(Cl)c(Cl)c5F)ncnc4cc3OC)C[C@H]2C1 ; C=CC(=O)N1C[C@H]2C[C@H](Oc3cc4c(Nc5ccc(Cl)c(Cl)c5F)ncnc4cc3OC3CC3)C[C@H]2C1 ; C=CC(=O)N1C[C@H]2C[C@H](Oc3cc4c(Nc5ccc(Cl)c(Cl)c5F)ncnc4cc3OC3CCOCC3)C[C@H]2C1 ; C=CC(=O)N1C[C@H]2C[C@H](Oc3cc4c(Nc5ccc(Cl)c(Cl)c5F)ncnc4cc3OCCOC)C[C@H]2C1 ; C=CC(=O)N1C[C@H]2C[C@H](Oc3cc4c(Nc5ccc(Cl)c(Cl)c5F)ncnc4cc3OCCCN3CCOCC3)C[C@H]2C1]

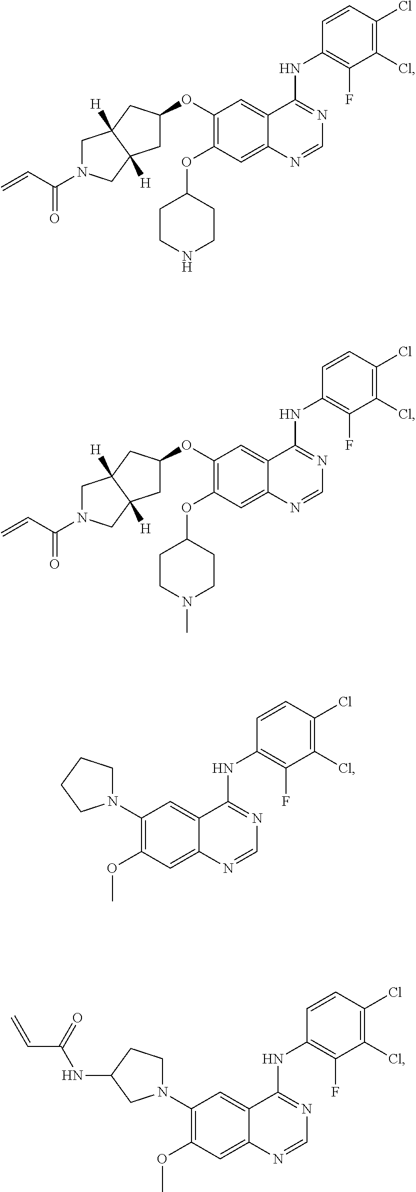 [SMILES: C=CC(=O)N1C[C@H]2C[C@H](Oc3cc4c(Nc5ccc(Cl)c(Cl)c5F)ncnc4cc3OC3CCNCC3)C[C@H]2C1 ; C=CC(=O)N1C[C@H]2C[C@H](Oc3cc4c(Nc5ccc(Cl)c(Cl)c5F)ncnc4cc3OC3CCN(C)CC3)C[C@H]2C1 ; COc1cc2ncnc(Nc3ccc(Cl)c(Cl)c3F)c2cc1N1CCCC1 ; C=CC(=O)NC1CCN(c2cc3c(Nc4ccc(Cl)c(Cl)c4F)ncnc3cc2OC)C1]

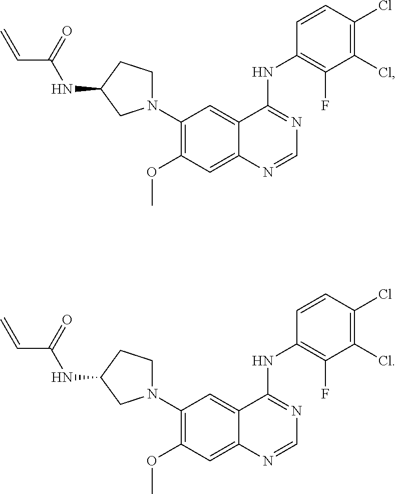 [SMILES: C=CC(=O)N[C@H]1CCN(c2cc3c(Nc4ccc(Cl)c(Cl)c4F)ncnc3cc2OC)C1 ; C=CC(=O)N[C@@H]1CCN(c2cc3c(Nc4ccc(Cl)c(Cl)c4F)ncnc3cc2OC)C1]

16. The compound used as the kinase inhibitor according to claim 7, wherein the compound is selected from the following compounds:

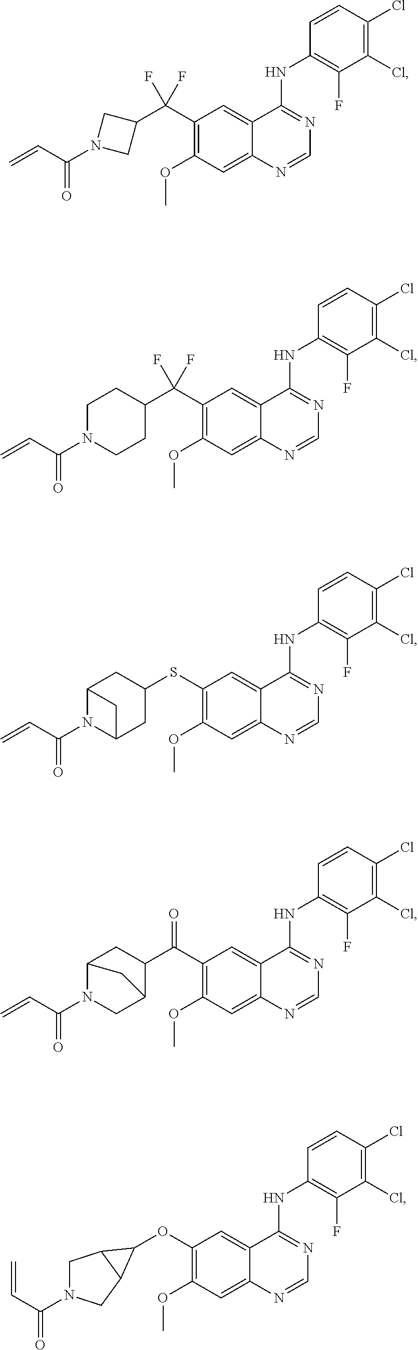 [SMILES: C=CC(=O)N1CC(C(F)(F)c2cc3c(Nc4ccc(Cl)c(Cl)c4F)ncnc3cc2OC)C1 ; C=CC(=O)N1CCC(C(F)(F)c2cc3c(Nc4ccc(Cl)c(Cl)c4F)ncnc3cc2OC)CC1 ; C=CC(=O)N1C2CC(Sc3cc4c(Nc5ccc(Cl)c(Cl)c5F)ncnc4cc3OC)CC1C2 ; C=CC(=O)N1CC2CC1CC2C(=O)c1cc2c(Nc3ccc(Cl)c(Cl)c3F)ncnc2cc1OC ; C=CC(=O)N1CC2C(C1)C2Oc1cc2c(Nc3ccc(Cl)c(Cl)c3F)ncnc2cc1OC]

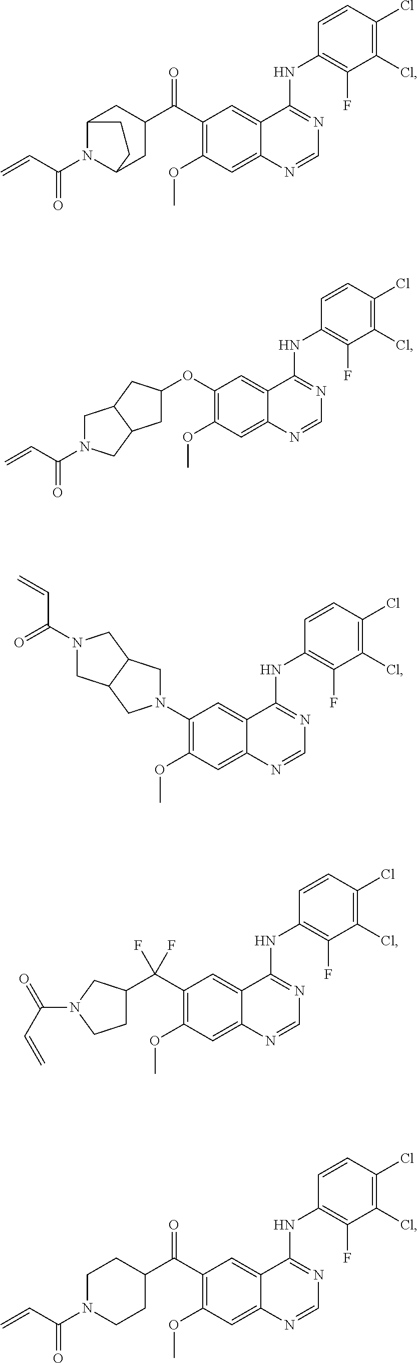 [SMILES: C=CC(=O)N1C2CCC1CC(C(=O)c1cc3c(Nc4ccc(Cl)c(Cl)c4F)ncnc3cc1OC)C2 ; C=CC(=O)N1CC2CC(Oc3cc4c(Nc5ccc(Cl)c(Cl)c5F)ncnc4cc3OC)CC2C1 ; C=CC(=O)N1CC2CN(c3cc4c(Nc5ccc(Cl)c(Cl)c5F)ncnc4cc3OC)CC2C1 ; C=CC(=O)N1CCC(C(F)(F)c2cc3c(Nc4ccc(Cl)c(Cl)c4F)ncnc3cc2OC)C1 ; C=CC(=O)N1CCC(C(=O)c2cc3c(Nc4ccc(Cl)c(Cl)c4F)ncnc3cc2OC)CC1]

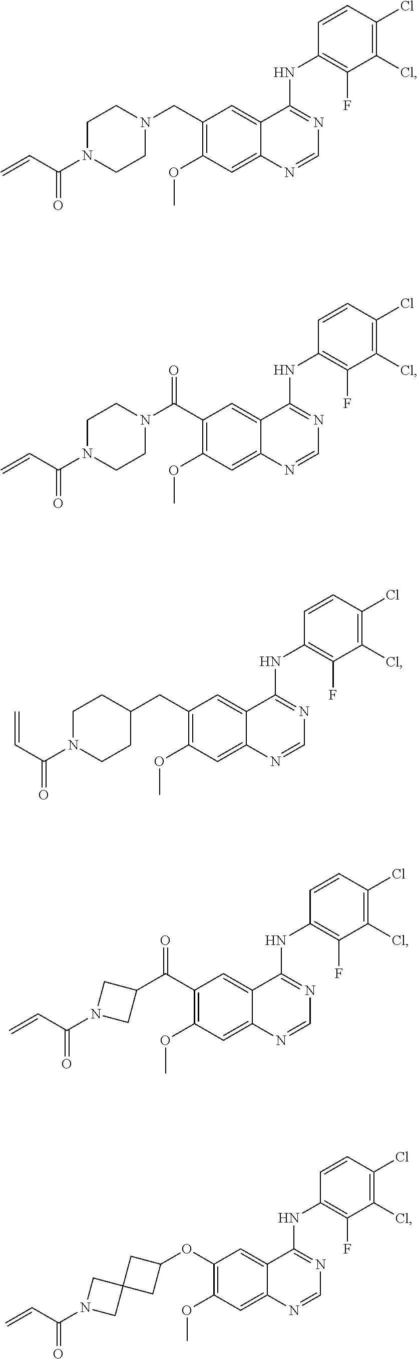 [SMILES: C=CC(=O)N1CCN(Cc2cc3c(Nc4ccc(Cl)c(Cl)c4F)ncnc3cc2OC)CC1 ; C=CC(=O)N1CCN(C(=O)c2cc3c(Nc4ccc(Cl)c(Cl)c4F)ncnc3cc2OC)CC1 ; C=CC(=O)N1CCC(Cc2cc3c(Nc4ccc(Cl)c(Cl)c4F)ncnc3cc2OC)CC1 ; C=CC(=O)N1CC(C(=O)c2cc3c(Nc4ccc(Cl)c(Cl)c4F)ncnc3cc2OC)C1 ; C=CC(=O)N1CC2(CC(Oc3cc4c(Nc5ccc(Cl)c(Cl)c5F)ncnc4cc3OC)C2)C1]

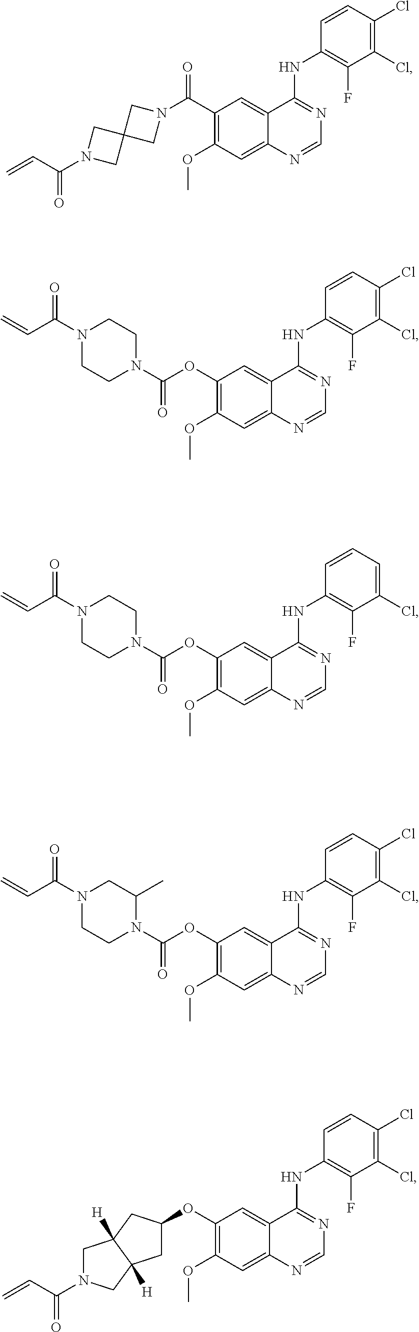 [SMILES: C=CC(=O)N1CC2(C1)CN(C(=O)c1cc3c(Nc4ccc(Cl)c(Cl)c4F)ncnc3cc1OC)C2 ; C=CC(=O)N1CCN(C(=O)Oc2cc3c(Nc4ccc(Cl)c(Cl)c4F)ncnc3cc2OC)CC1 ; C=CC(=O)N1CCN(C(=O)Oc2cc3c(Nc4cccc(Cl)c4F)ncnc3cc2OC)CC1 ; C=CC(=O)N1CCN(C(=O)Oc2cc3c(Nc4ccc(Cl)c(Cl)c4F)ncnc3cc2OC)C(C)C1 ; C=CC(=O)N1C[C@H]2C[C@H](Oc3cc4c(Nc5ccc(Cl)c(Cl)c5F)ncnc4cc3OC)C[C@H]2C1]

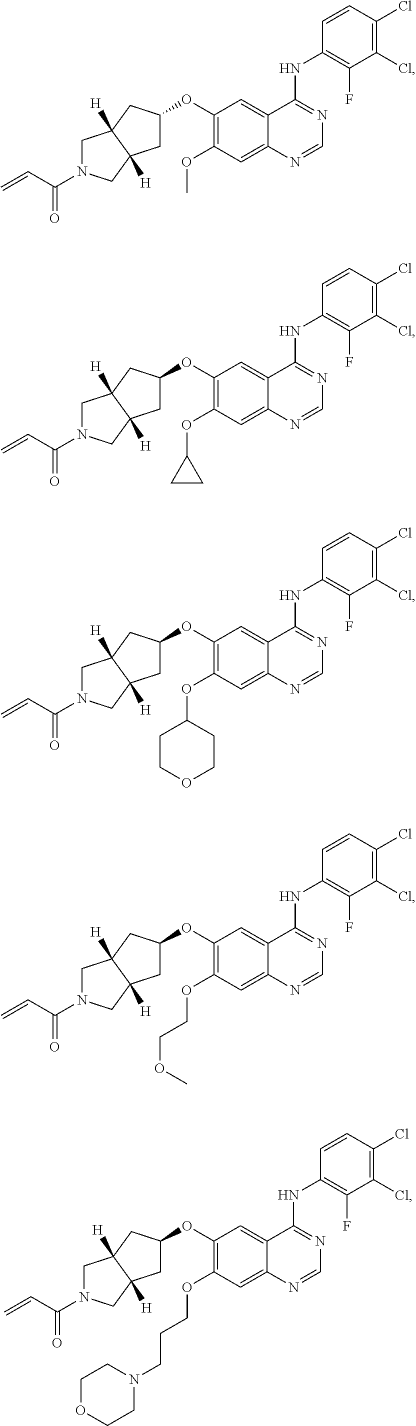 [SMILES: C=CC(=O)N1C[C@H]2C[C@@H](Oc3cc4c(Nc5ccc(Cl)c(Cl)c5F)ncnc4cc3OC)C[C@H]2C1 ; C=CC(=O)N1C[C@H]2C[C@H](Oc3cc4c(Nc5ccc(Cl)c(Cl)c5F)ncnc4cc3OC3CC3)C[C@H]2C1 ; C=CC(=O)N1C[C@H]2C[C@H](Oc3cc4c(Nc5ccc(Cl)c(Cl)c5F)ncnc4cc3OC3CCOCC3)C[C@H]2C1 ; C=CC(=O)N1C[C@H]2C[C@H](Oc3cc4c(Nc5ccc(Cl)c(Cl)c5F)ncnc4cc3OCCOC)C[C@H]2C1 ; C=CC(=O)N1C[C@H]2C[C@H](Oc3cc4c(Nc5ccc(Cl)c(Cl)c5F)ncnc4cc3OCCCN3CCOCC3)C[C@H]2C1]

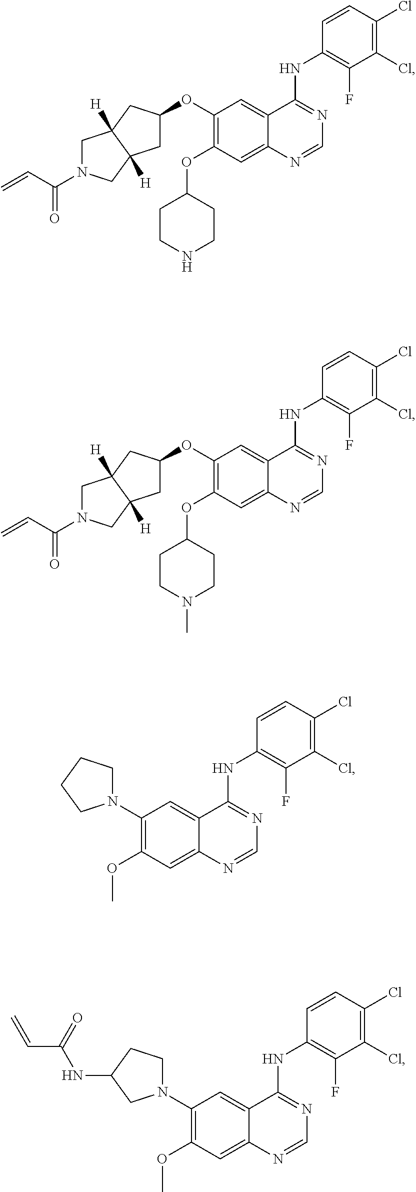 [SMILES: C=CC(=O)N1C[C@H]2C[C@H](Oc3cc4c(Nc5ccc(Cl)c(Cl)c5F)ncnc4cc3OC3CCNCC3)C[C@H]2C1 ; C=CC(=O)N1C[C@H]2C[C@H](Oc3cc4c(Nc5ccc(Cl)c(Cl)c5F)ncnc4cc3OC3CCN(C)CC3)C[C@H]2C1 ; COc1cc2ncnc(Nc3ccc(Cl)c(Cl)c3F)c2cc1N1CCCC1 ; C=CC(=O)NC1CCN(c2cc3c(Nc4ccc(Cl)c(Cl)c4F)ncnc3cc2OC)C1]

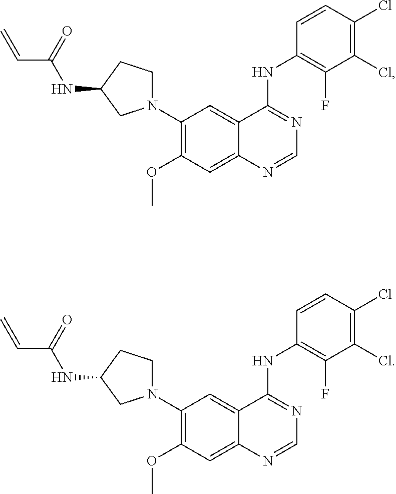 [SMILES: C=CC(=O)N[C@H]1CCN(c2cc3c(Nc4ccc(Cl)c(Cl)c4F)ncnc3cc2OC)C1 ; C=CC(=O)N[C@@H]1CCN(c2cc3c(Nc4ccc(Cl)c(Cl)c4F)ncnc3cc2OC)C1]

17. Use of the compound used as kinase inhibitor according to claim 5 in the preparation of medicine, wherein the medicine is used for treatment of related diseases caused by EGFR mutation and/or Her2 mutation.

18. Use of the compound used as kinase inhibitor according to claim 6 in the preparation of medicine, wherein the medicine is used for treatment of related diseases caused by EGFR mutation and/or Her2 mutation.

19. Use of the compound used as kinase inhibitor according to claim 7 in the preparation of medicine, wherein the medicine is used for treatment of related diseases caused by EGFR mutation and/or Her2 mutation.

20. Use of the compound used as kinase inhibitor according to claim 8 in the preparation of medicine, wherein the medicine is used for treatment of related diseases caused by EGFR mutation and/or Her2 mutation.
